SEQUENCE LISTING (190 sequences)

SEQ ID NO: 1 - protein, 124 aa, Lama glama
EVQLVESGGGLVQAGGSLRLSCAASGFTFDDYALGWFRQAAGKEREGVSCIRCSDGSTYYADSVKGRFTISSDNAKNTVYLQMNSLKPEDTAVYYCAASIVPRSKLEPYEYDAWGQGTQVTVSS

SEQ ID NO: 2 - protein, 111 aa, Lama glama
EVQLVESGGGLVQPGGSLRLSCVHSGTISSTHAMGWYRQAPGKQREHVATFTNRGSTYYAGSVKGRFTISRDNAKNTMYLQMNSLKPEDTAVYYCNTGPYWGQGTQVTVSS

SEQ ID NO: 3 - protein, 120 aa, Lama glama
EVQLVESGGGLVQAGGSLRLSCAASGRTFSSSVMGWFRQAPGKEREFVAAISGSGSSTDSAQGRFTISRDNAKNTVYLQMNSLKPEDTAVYYCAAGRAFLARDTFYYDIWGQGTQVTVSS

SEQ ID NO: 4 - protein, 126 aa, Lama glama
EVQLVESGGGLVQAGGSLRLSCAASGFAFDDHLIGWFRQAPGKEREAVSCISRSAGSTYYADSVKGRFTISSDNAKNTVYLQMNSLKADDTAVYYCAAGPAWGRPASPLPYEYDYWGQGTQVTVSS

SEQ ID NO: 5 - protein, 134 aa, Lama glama
EVQLVESGGGLVQAGGSLRLSCAASGRTISSYAMAWFRQAPGKEREFVAAISLSGDSTYYADSVKGRFTISRDNAKNTVYLQMNSLKPEDTAEYYCAATDWDFEDIPEYYCSGYGCDESLFDSWGQGTQVTVSS

SEQ ID NO: 6 - protein, 130 aa, Lama glama
EVQLVESGGGLVQPGGSLRLSCAASGFTLDYDAIGWFRQAPGKEREGVSCITSSDGITYYADSVKGRFTISRDNAKNTVYLQMNSLKPEDTAVYYCATGNRRIYYSDYALACFPYEYDYWGQGTQVTVSS

SEQ ID NO: 7 - protein, 123 aa, Lama glama
EVQLVESGGGLVQVGDSLRLSCAASGRTFSTYLMVGWFRQAPGKEREFAAVIWSSGDTAYADSVKGRFTISRDSAKNTVYLQMNSLKAEDTAVYYCAGSYDGNYYIPGFYKDWGQGTQVTVSS

SEQ ID NO: 8 - protein, 130 aa, Lama glama
EVQLVESGGGLVQPGGSLRLSCTTSGFTLDYYAVGWFRQAPGKEREGVSCISSSDGSTYYADSVKGRFTISRDNAKNTVYLQMNSLKPEDTAVYYCATGNLRIYYSDYALACFPYEYDYWGQGTQVTVSS

SEQ ID NO: 9 - protein, 123 aa, Lama glama
EVQLVESGGGLVQVGDSLRLACAASGRTFSTYLMVGWFRQAPGKEREFAAGIWSSGDTAYADSVKGRFTISRDNAKNTVYLQMNSLKTEDTAVYYCAGSYDGNYYIPGFYKDWGQGTQVTVSS

SEQ ID NO: 10 - protein, 123 aa, Lama glama
EVQLVESGGGLVQVGDSLRLSCAASGRTFSTYLMVGWFRQAPGKEREFAAAMWSSGVPAYADSVKGRFTISRDNAKNTVYLQMNSLKAEDTAVYYCAGSYGGNYYIPGFYEDWGQGTQVTVSS

SEQ ID NO: 11 - protein, 124 aa, Lama glama
EVQLVESGGGLVQAGGSLRLSCAASGHTFSRYAMGWFRRVPGKEREFVTHITWNRGSTYYADSVKGRFTISRDKASNTLYLQMNSLKPEDTAVYYCAAQIKYGAVTHPEEYSYWGQGTQVTVSS

SEQ ID NO: 12 - protein, 126 aa, Lama glama
KVQLVESGGGLVQAGGSLRLSCAASGFAFDDHLIGWFRQAPGKEREAVSCISRSAGSTYYADSVKGRFTISSDNAKNTVYLQMNSLKADDTAVYYCAAGPAWGRPASPLPYEYDYWGQGTQVTVSS

SEQ ID NO: 13 - protein, 134 aa, Lama glama
EVQLVESGGGLVQAGGSLRLSCAASGFTFDDYAIGWFRQAPGKEREGVSCISSSDGSTAYADSVKGRFTISSDNAKNTVYLQMNSLKPEDTAVYYCTAVPATRRTPQMVAANVCWLAEYEYDYWGQGTQVTVSS

SEQ ID NO: 14 - protein, 123 aa, Lama glama
EVQLVESGGGLVQVGDSLRLSCAASGRTFSTYLMVGWFRQAPGKEREFAAGIWSSGGTAYADSVKGRFTVSRDNAKNTVYLQMNSLKAEDTAVYYCAGSYDGNYYIPGFYKDWGQGTQVTVSS

SEQ ID NO: 15 - protein, 123 aa, Lama glama
EVQLVEFGGGLVQVGDSLRLSCAASGRTFSTYLMVGWFRQAPGKEREFAAGIWSSGGTAYADSVKERFTVSRDNDKNTVYLQMNSLQAEDTAVYYCAGSYDGNYYIPGFYKDWGQGTQVTVSS

SEQ ID NO: 16 - protein, 123 aa, Lama glama
EVQLVESGGGLVQVGDSLRLSCAASGHTFSTYLMVGWFRQTPGKEREFAAVIWSSGDTAYADSVKGRFTISRDNAKNTVYLQMNSLKAEDTAVYYCAGSYGGNYYIPGFYEDWGQGTQVTVSS

SEQ ID NO: 17 - protein, 126 aa, Lama glama
EVQLVESGGGLVQPGGSLRLSCAASGFALDYYAIGWFRQVPGKEREGVSCISSSDGITYYVDSVKGRFTISRDNAKNTVYLQMNSLKPEDTAVYYCATDSGGYIDYDCMGLGYDYWGQGTQVTVSS

SEQ ID NO: 18 - protein, 124 aa, Lama glama
EVQLVESGGGLVQAGGSLRLSCAASGRTFSSYAMGWFRQAPGKELEFVTAVSWSGGSTYYADSVKGRFTISRDSAKNTVYLQMNSLKPEDTAVYYCAAQSTIVEVTTLEAYDYWGQGTQVTVSS

SEQ ID NO: 19 - protein, 122 aa, Lama glama
EVQLVESGGGSVQAGGSLRLSCAASERTFSPYAMGWFRQAPGKEREFVAHITWSAGSTYYADSVKGRFTISRDNAKNTVYLQMNSLKPEDTAVYYCAAKRRYGIVDRDYNDWGQGTQVTVSS

SEQ ID NO: 20 - protein, 116 aa, Lama glama
EVQLVESGGGLVQAGGSLRLACIASGRDIFSVSATGWYRQAPGKQREFVAGISNIGATKFADSVKGRFTISRDNAKNTVYLQMNSLKPEDTAVYYCNVLLWSGNLWGQGTQVTVSS

SEQ ID NO: 21 - protein, 116 aa, Lama glama
EVQLVESGGGLVQAGGSLRLSCIASGRDIFSITAIGWYRQAPGKQREFVAGISNIGATKYTDSVKGRFTISGDNAENTVYLQMNSLKPEDTAVYYCNVLLWSANYWGQGTQVTVSS

SEQ ID NO: 22 - protein, 126 aa, Lama glama
EVQLVESGGGLVQAGGSLRLSCAASGFAFDDHLIGWFRQAPGKEREAVSCISRSAGSTYYADSVKGRFTISGDNAKNTVYLQMNSLKADDTAVYYCAAGPAWGRPASPLPYEYDYWGQGTQVTVSS

SEQ ID NO: 23 - protein, 134 aa, Lama glama
EVQLVESGGGLVQAGGSLRLSCAASGFTFDDYAIGWFRQAPGKEREGVSCISSSDGSTAYADSVKGRFTISSDNAKNTVYLQMNSLKPEDTAVYYCTAVPATRRTPQMVVANVCWLAEYEYDYWGQGTQVTVSS

SEQ ID NO: 24 - protein, 125 aa, Lama glama
EVQLVESGGGLVQAGGSLRLSCVASGFTFDDYAIGWFRQAPGKEREGVSCISSSDGSTYYADSVKGRFTISGDNAKNTVYLQMNSLKPEDTAVYYCAAGITPCSDYTQTYEYDVWGQGTQVTVSS

SEQ ID NO: 25 - protein, 134 aa, Lama glama
EVQLVESGGGLVQAGGSLRLSCAASGFTFDDYAIGWFRQAPGKEREGVSCISRSDGSTSYADSVKGRFTISSDNAKNTVYLVMNSLKPEDTAVYYCTAVPATRRTPQMVVANMCWLAEYEYDYWGQGTQVTVSS

SEQ ID NO: 26 - protein, 125 aa, Lama glama
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCAAGITPCSDYTQTYEYDVWGQGTQVTVSS

SEQ ID NO: 27 - protein, 134 aa, Lama glama
EVQLVESGGGLVQAGGSLRLSCAVSGFTFDDYAIGWFRQAPGKEREGVSCISSSDGSTAYADSVKGRFTISSDNAKNTVYLQMNSLKPEDTAVYYCTAVPATRRTPQMVVANVCWLAEYEYDYWGQGTQVTVSS

SEQ ID NO: 28 - protein, 132 aa, Lama glama
EVQLVESGGGLVQAGGSLRLSCAASGFTFDDYAIGWFRQAPGKEREGVSYISSSDGSTYYADSVKGRFTSSSDNAKNTVYLQMNSLKPEDTAVYYCAARPTLRVRLDNDRHHLLYEYEYDYWGQGTQVTVSS

SEQ ID NO: 29 - protein, 134 aa, Lama glama
EVQLVESGGGLVQAGGSLRLSCAASGFTFDDYTIGWFRQAPGKEREGVSCISSSDGSTSYADSVKGRFTISSDNAKNTVYLQMNSLKPEDTAVYYCTAVPATRRTPQMVVLNMCWLAEYEYDYWGQGTQVTVSS

SEQ ID NO: 30 - protein, 125 aa, Lama glama
EVQLVESGGGLVQAGGSLRLSCVASGFTFDDYAIGWFRQAPGKEREGVSCISSSDGSTYYADSVKGRFTISSDNAKNTVYLQMNSLKPEDTAVYYCAAGITPCSDYTQTYEYDVWGQGTQVTVSS

SEQ ID NO: 31 - protein, 134 aa, Lama glama
EVQLVESGGGSVQAGGSLRLSCAASGFTFDDYAIGWFRQAPGKEREGVSCISSSDGSTNYADSVKGRFTISSDTAKNTVYLQMNSLKPEDTAVYYCTAVPATRRTPQMVDANMCWLAEYEYDYWGQGTQVTVSS

SEQ ID NO: 32 - protein, 127 aa, Lama glama
EVQLVESGGGLAQAGGSLRLSCAASGRRFGGHAMGWFRQAPGEEREFVATIYWTSGMTRYAGSVKGRFTISRDNAENTVFLQMNSLKPEDTAVYYCAVIKDFQLRVDVTSASAYDYWGQGTQVTVSS

SEQ ID NO: 33 - protein, 127 aa, Lama glama
GVQLVESGGGLAQAGGSLRLSCAASGRRFGGHAMGWFRQAPGKDREFVATIYWTTGMTRYADSVKGRFTISRDNAENTVFLQMNSLKPEDTAVYYCALIRDFNIRLDVTSASAYGYWGQGTQVTVSS

SEQ ID NO: 34 - protein, 123 aa, Lama glama
EVQLVESGGGLVQVGDSLRLSCAASGRTFSTYLMVGWFRQAPGKEREFAAGIWSSGDTAYADSVRGRFTISRDNAKNTVYLQMNSLKTEDTAVYYCAGSYDGNYYIPGFYKDWGQGTQVTVSS

SEQ ID NO: 35 - protein, 123 aa, Lama glama
EVQLVESGGGLVQVGDSLRLSCAASGRTFSTYLVVGWFRQAPGKEREFAAAIWSSGDTAYADSVKGRFTISRDNAKNTVYLQMNSLKAEDTAVYYCAGSYGGNYYIPGFYEDWGQGTQVTVSS

SEQ ID NO: 36 - protein, 121 aa, Lama glama
EVQLVESGGGLVQAGGSLRLSCAASGRTFSRNAMAWFRQVPGKVREFVAGIRWNVGRLDYADSVKGRFTISRDNAENTVYLQMNDLKTEDTAVYYCAAYAGLVFSGIPDYWGQGTQVTVSS

SEQ ID NO: 37 - protein, 118 aa, Lama glama
EVQLVESGRGLVQAGGSLRLSCAASGSIFSINAMLWYRQAPGKQRELVAAITSGGSTNYADSVKGRFTISRDNAKNTVYLQMNSLKPEDTAVYYCAADSDYSSDYYYWGQGTQVTVSS

SEQ ID NO: 38 - protein, 133 aa, Lama glama
EVQLVESGGGLVQSGGSLRLSCAASGFALDYYTIGWFRQAPGKEREGVSCISGGDTSTYYADSVKGRFTISRDNAKNTVYLQMNSLKPEDTAVYYCATDSAGVPAGPAAVYGSTCSRLEYDYWGQGAQVTVSS

SEQ ID NO: 39 - protein, 124 aa, Lama glama
EVQLVESGGGLVQAGGSLRLSCAASGGTFSSYSMGWFRQAPGKEREFIAAINWNGDSTYYEDSVKGRFTVSRDNAKNTVYLQMNSLKPEDTAVYYCAATGWGRAYEQAYEYDVWGQGTQVTVSS

SEQ ID NO: 40 - protein, 119 aa, Lama glama
EVQLVESGGGLVQPGGSLRLSCAASGFTVDDYAMSWVRQAPGKGLEWVSTISWNDEYTYYAESMKGRFTISRDNAKNTLYLQMNSLKSEDTAVYYCAKGGSRLYDYHYWGQGTQVTVSS

SEQ ID NO: 41 - protein, 123 aa, Lama glama
EVQLVESGGGLVQAGGSLRLSCASSGFTFDDYAIGWFRQAPGKEREGVSCISSSDGSTYYADSVKGRFTISSDNAKNTVYLQMNSLKPEDTAVYYCAARPPFHSCSEYENDYWGQGTQVTVSS

SEQ ID NO: 42 - protein, 120 aa, Lama glama
EVQLVESGGGLVQAGGSLRLSCAASGRTISSSVLGWFRQAPGKEREFVAAISGSGSSTDSAKDRFTISRDNAKNTVYLQMNSLKPEDTAVYYCAAGRAFLTRDPFYYDIWGQGTQVTVSS

SEQ ID NO: 43 - protein, 116 aa, Lama glama
EVQLVESGGGLVQAGGSLRLTCIASGRDIFSVTAMGWYRQAPGKQREFVAGLSNIGATKYADSVKGRFTVSGDAAKNTVYLQMNSLKPEDTAVYYCNVLLWSGNYWGQGTQVTVSS

SEQ ID NO: 44 - protein, 132 aa, Lama glama
EVQLVESGGGLVQAGGSLRLSCAASGFTFDDYAIGWFRQAPGKEREGVSYISKSDGTTYYADSVKGRFTSSSDNAKNTVYLQINSLKPEDTAVYYCAARPTLRVRLDNDRHHLLYEYEYDYWGQGTQVTVSS

SEQ ID NO: 45 - protein, 132 aa, Lama glama
EVQLVESGGGLVQAGGSLRLSCAASGFTFDEYAIGWFRQAPGKEREGVSYISSSDGSTYYADSVKGRVTSSSDNAKNTVYLQMNSLKPEDTAVYYCAARPTLRVRLDNDRHHLLYEYEYDYWGQGTQVTVSS

SEQ ID NO: 46 - protein, 124 aa, Lama glama
EVQLVESGGGLVQAGGSLRLSCAASGRTFSSYAMGWFRQAPGKEREFVARISWNGGSTYHADSVKGRFTISRDNPKNTVYLQMDSLKPEDAAIYYCAASIALVGGVTPHSYDYWGQGTQVTVSS

SEQ ID NO: 47 - protein, 123 aa, Lama glama
EVQLVESGGGLVQVGDSLRLSCAASGRTFSTYLMVGWFRQAPGKEREFAAVIWSSGDTDYADSVKGRFTISRDNAKNTVYLQMNSLKAEDTAVYYCAGSYGGNYYIPGFYKDWGQGTQVTVSS

SEQ ID NO: 48 - protein, 123 aa, Lama glama
EVQLVESGGGLVQVGDSLRLSCAASGRTFSTYLMVGWFRQAPGKEREFAAGIWSSGGTAYADSVKGRFTISRDNAKNTVYLQMNSLKAEDTAVYYCAGSYDGNYYIPGFYKDWGQGTQVTVSS

SEQ ID NO: 49 - protein, 128 aa, Lama glama
EVQLVESGGGLVQAGGSLGLSCAASERTLPSYVMGWFRQAPGKELEFVAGISWSSGRTYYTDSVKGRFTISRDAAENTWYLQMNSLKPEDTAVYYCASNSVSEPTLHTWQYEASYDYWGQGTQVTVSS

SEQ ID NO: 50 - protein, 124 aa, Lama glama
EVQLVESGGGLVQAGGSLRLSCAASGHTFSRYAMGWFRRVPGEEREFVTHITWNRGSTYYADSVKGRFTISRDKASNTLYLQMNSLKPEDTAVYYCAAQIKYGEITHPEEYSYWGQGTQVTVSS

SEQ ID NO: 51 - protein, 121 aa, Lama glama
EVQLVESGGGLVQAGGSLRLSCAASGRTFSRNAMGWFRQVPGKAREFVAAIRWNVGRLDYADSVKGRFAISRDNAENTVYLQMNDLKTEDTAVYYCAAYAGLVYSGIPDYWGQGTQVTVSS

SEQ ID NO: 52 - protein, 124 aa, Lama glama
EVQLVESGGGLVQAGGSLRLSCAASGHTFSRYAMGWFRRVPGKEREFVTHITWNRGSTYYADSVKGRFTISRDKASNTLYLQMNSLKPEDTAVYYCAAQIKYGEITHPEEYSYWGQGTQVTVSS

SEQ ID NO: 53 - protein, 124 aa, Lama glama
EVQLVESGGGLVQAGDSLRLSCAASGHTFSRYAMGWFRRVPGKEREFVTHITWNRGSTYYADSVKGRFTISRDKASNTLYLQMNSLKSEDTAVYYCAAQTKYGEITRPEEYSYWGQGTQVTVSS

SEQ ID NO: 54 - protein, 115 aa, Lama glama
EVQLVESGGGLVQAGGSLRLVCSASGIDFSINAMAWYRQAPGKQREWVAFMINDSSTDYTDSVKGRFTISRDSTKNILYLQMNNLNVEDTAVYYCNTAYEQHTYWGQGTQVTVSS

SEQ ID NO: 55 - protein, 128 aa, Lama glama
EVQLVESGGGLVQAGGSLRLSCAASGFTFDDYAIAWFRQAPGKEREGISCITPSDDRTYYADSVKGRFIISSDNAKNTVYLQMNSLKPEDTADYYCAAVPRLRGLGYWPYPEYEYDYWGQGTQVTVSS

SEQ ID NO: 56 - protein, 124 aa, Lama glama
KVQLVESGGGLVQAGGSLRLSCAASGFTFDDYALGWFRQAAGKEREGVSCIRCSDGSTYYADSVKGRFTISSDNAKNTVYLQMNSLKPEDTAVYYCAASIVPRSKLEPYEYDAWGQGTQVTVSS

SEQ ID NO: 57 - protein, 119 aa, Lama glama
EVQLVESGGGLVQPGGSLRLSCAVSGFTVDDYAMSWVRQAPGKGLEWVSTISWNDEYTYYAESMKGRFTISRDNAKNTLYLQMNSLKSEDTAVYYCAKGGSRLYDYHYWGQGTQVTVSS

SEQ ID NO: 58 - protein, 131 aa, Lama glama
EVQLVESGGGLVQAGGSLRLSCAASGFTLDDYAIGWFRQAPGKEREGVSCISSSDGSTTYADSVKGRFTVSSDNAKNTVYLQMNSLKPEDTAVYYCAAGLRGRYYRGTYSLVCAPYEYDFWGQGTQVTVSS

SEQ ID NO: 59 - protein, 131 aa, Lama glama
EVQLVESGGGLVQAGGSLRLSCAASGFTLDDYAIGWFRQAPGKEREGVSCISSSDGSTYYADSVKGRFTISADNAKNTVYLQMHSLKPEDTAVYYCAAGLRGRYYSGSNYLVCAPYEYDYWGQGTQVTVSS

SEQ ID NO: 60 - protein, 125 aa, Lama glama
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCAADFIISSKRLCLDLFGSRGQGTQVTVSS

SEQ ID NO: 61 - protein, 125 aa, Lama glama
EVQLVESGGGSVQAGGSLRLSCAASGFTFDDYAIGWFRQAPGKEREGVSCISSSDGSTYYADSVKGRFTISSDNAKNTVYLQMNSLKPGDTAVYYCAAGITPCSDYTQTYEYDVWGQGTQVTVSS

SEQ ID NO: 62 - protein, 134 aa, Lama glama
EVQLVESGGGLVQAGGSLRLSCAASGFTFDDYAIGWFRRAPGKEREGVSCITSSDGSTSYADSVKGRFTISSDSAKNTVYLQMNSLKPEDTAVYYCSVVPATRRNPQMVVAKKCWLAEYEYDYWGQGTQVTVSS

SEQ ID NO: 63 - protein, 130 aa, Lama glama
EVQLVESGGGLVQAGGSLRLSCAVSGFTFDDYAIGWFRQAPGKEREGVSCISRSDGSPYYADSVKGRFTISSDNAKGTVYLQMSSLKPEDTAVYYCAASWSGAYYSGTYYCDRLYEYDAWGQGTQVTVSS

SEQ ID NO: 64 - protein, 123 aa, Lama glama
EVQLVESGGGLVQVGDSLRLSCAASERTFSTYLMVGWFRQAPGKEREFAATMWSSGDTAYADSVKGRFTISRDNAKNTVYLQMNSLKAEDTAVYYCAGSYGGNYYIPGFYEDWGQGTQVTVSS

SEQ ID NO: 65 - protein, 127 aa, Lama glama
EVQLVESGGGLVQPGGSLRLSCAASGFALDYYAIGWFRQAPGKEREGVSCISSSDGSTYYADSVKGRFTISRDNAKNTVYLQMNSLKPEDTAVYYCATDSLGYGSSCRMAPYEYDYWGQGTQVTVSS

SEQ ID NO: 66 - protein, 123 aa, Lama glama
EVQLVESGGGLVQVGDSLRLSCAASGRTFSTYLMVGWFRQAPGKEREFAAVIWSSGGTAYADSVKGRFTISRDNAKNTVYLQMNSLKAEDTAVYYCAGSYDGNYYIPGFYKDWGQGTQVTVSS

SEQ ID NO: 67 - protein, 130 aa, Lama glama
EVQLVESGGGLVQPGGSLRLSCAASGFTSDDYAIGWFRQAPGKEREGVSCISSSDGITYYADSVKGRFTISRDNAKNTVYLQMNSLKPEDTAVYYCATGNRRIYYSDYALACFPYEYDYWGQGTQVTVSS

SEQ ID NO: 68 - protein, 123 aa, Lama glama
EVQLVESGRRLVQVGDSLRLACAASGRTFSTYLMVGWFRQAPGKEREFAAGIWSSGDTAYADSVKGRFTISRDNAKNTVYLQMNSLKTEDTAVYYCAGSYDGNYYIPGFYKDWGQGTQVTVSS

SEQ ID NO: 69 - protein, 123 aa, Lama glama
EVQLVESGGGLVQVGDSLRLSCAASERTFSTYLMVGWFRQAPGKEREFAATMWVSGDTAYADSVKGRFTISRDNPKNTVYLQMNSLKAEDTAVYYCAGSYGGNYYIPGFYKDWGQGTQVTVSS

SEQ ID NO: 70 - protein, 123 aa, Lama glama
EVQLVESGGGLVQIGDSLRLSCAASGRTFSTYLMVGWFRQAPGKEREFAAAIWSSGDTAVADSVKGRFTISRDNAKNTVYLQMNSLKAEDTAVYYCAGSYGGNYYIPGFYEDWGQGTQVTVSS

SEQ ID NO: 71 - protein, 122 aa, Lama glama
EVQLVESGGGLVQPGGSLRLSCAASGFTLDYYAIGWFRQAPGKEWEGVSCISSSDGSTYYADSVKGRFTISRDNAKNTVYLQMNSLKPEDTAVYYCATDSIVCGSYYGMDYWGKGTQVTVSS

SEQ ID NO: 72 - protein, 123 aa, Lama glama
EVQLVESGGASVQPGGSLRLSCAASGRTFSTYLMVGWFRQAPGKEREFAAGIWSSGGTAYADSVKERFTVSRDNDKNTVYLQMNSLQAEDTAVYYCAGSYDGNYYIPGFYKDWGQGTQVTVSS

SEQ ID NO: 73 - protein, 128 aa, Lama glama
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCAAAWGASRLPIGTMPPYEYDYWGQGTQVTVSS

SEQ ID NO: 74 - protein, 118 aa, Lama glama
EVQLVESGGGLVQAGGSLRLVCSASGIDFSINVMGWYRQAPGKQREWVAFIGSGGSTDYIDYTDSVKGRFTISRDSTKNILYLRMNNLNVEDTAVYYCNTAYEQHTYWGQGTQVTVSS

SEQ ID NO: 75 - protein, 125 aa, Lama glama
EVQLVESGGGLVQAGGSLRLSCAASGFTFDDYAIGWFRQAPGKDLEGVSCISSSDGSTYYADSVKGRFTISSDNAKNTVYLQMNSLKPEDTAVYYCAAVNGLGPFSVPVPVYDFWGQGTQVTVSS

SEQ ID NO: 76 - protein, 130 aa, Lama glama
EVQLVESGGGLVQAGGSLRLSCAVSGLPFDDYVIGWFRQAPGKEREGVSCISSSDGSTYYADSVKGRFTISSDNAKNTVYLQMNSLKPEDTAAYYCAAGGPRINIATMTCSHDEYEYDYWGQGTQVTVSS

SEQ ID NO: 77 - protein, 133 aa, Lama glama
EVQLVESGGGLVQPGGSLRLSCAASGFALDYYAIGWFRQAPGKEREGVSCIESSDGSTYYADSVKGRFTISRDNAKNTVYLQMNSLKPEDTAVYYCATDSAGVPAGPAAVYGSSCSRLEYDYWGQGTQVTVSS

SEQ ID NO: 78 - protein, 124 aa, Lama glama
EVQLVESGGGLVQPGGSLRLSCAASGFTFGSYDMSWVRQAPGKGPEWVSAINSRGGSTYYADSVKGRFTISRDNAKNTLYLQMNSLKPEDTAVYYCATDPYSLSYYGYPLYDYWGQGTQVTVSS

SEQ ID NO: 79 - protein, 115 aa, Lama glama
EVQLVESGGGLVQAGGSLRLVCSASGIDFSINVMGWYRQAPGKQREWVAFIGSGSSTGYTDSVKGRFSISRDSTKNILYLQMNNLNVEDTAVYYCNTAYEQHTYWGQGTQVTVSS

SEQ ID NO: 80 - protein, 129 aa, Lama glama
EVQLVESGGGLVQAGGSLRLSCAASGFTLDDYAIGWFRQAPGKEREGVSCIRDSDGSTYYADSVKGRFTISSDNDKNTVYLQMNSLKPEDTAVYYCAAVPAGRLRFGEQWYPLYEYDAWGQGTQVTVSS

SEQ ID NO: 81 - protein, 124 aa, Lama glama
EVQLVESGGGVVQAGDSVRLSCAASGPTFRSYTMAWFRQTPGKERDIVAAISSSLGRTYYADSVKGRFQILRDNAKETVWLQMNSLKPEDTAVYICAASRSLNLAYTTKPYDYWGQGTQVTVSS

SEQ ID NO: 82 - protein, 133 aa, Lama glama
EVQLVESGGGLVQAGGSLRLSCAASGFDFEDYDMGWFRQAPGKEREGVSYISSSDGSTYYTDSVKGRFTISSDNAKNTVYLQMNSLIPEDTAVYYCAARPWTRRVYGSSWLARSLDEYEYDYWGQGTQVTVSS

SEQ ID NO: 83 - protein, 123 aa, Lama glama
EVQLVESGGGLVQVGDSLRLSCAASGHTFSTYLMVGWFRQTPGKEREFAAVIWSSGDTAYADSAKGRFTISRDNAKNTVYLQMNSLKAEDTAVYYCAGSYGGNYYIPGFYEDWGQGTQVTVSS

SEQ ID NO: 84 - protein, 126 aa, Lama glama
EVQLMESGGGLVQPGGSLRLSCAASGFALDYYAIGWFRQVPGKEREGVSCISSSDGITYYVDSVKGRFTISRDNAKNTVYLQMNSLKPGDTAVYYCATDSGGYIDYDCMGLGYDYWGQGTQVTVSS

SEQ ID NO: 85 - protein, 126 aa, Lama glama
EVQLVESGGGLVQPGGSLRLSCAASGFALDYYAIGWFRQAPGKEREGVSCISSSDGITYYVDSVKGRFTISRDNAKNTVYLQMNSLKPEDTAVYYCATDSGGYIDYDCMGLGYDYWGQGTQVTVSS

SEQ ID NO: 86 - protein, 123 aa, Lama glama
EVQLVESGGGLVQVGDSLRLSCAASGRTFSTYLMVGWFRQAPGKEREFAAVIWSSGDTAYADSVKGRFTISRDNAKNTVYLQMNSLKAEDTAVYYCAGSYDGNYYIPGFYKDWGQGTQVTVSS

SEQ ID NO: 87 - protein, 129 aa, Artificial
  Mutated Lama sequence
EVQLVESGGGLVQAGGSLRLSCAASGFTLDDYAIGWFRQAPGKEREGVSSIRDNDGSTYYADSVKGRSTISSDNDKNTVYLQMDSLKPEDTAVYYCAAVPAGRLRYGEQWYPIYEYDAWGQGTLVTVSS

SEQ ID NO: 88 - protein, 129 aa, Artificial
  Mutated Lama sequence
EVQLVESGGGLVQAGGSLRLSCAVSGITLDDYAVGWFRQAPGKEREGVSTIRDNDGSTYYADSVKGRFTISSDNDKNTVYLQMNSLKPKDTAVYYCAAVPAGRLRFGEQWYPIYEYDAWGQGTLVTVSS

SEQ ID NO: 89 - protein, 129 aa, Artificial
  Mutated Lama sequence
EVQLVESGGGQAQAGGSLRLSCAVSGFTLDDYAIGWFRQAPGKEREGVSSIRDNDGSTYYTDSVKGRFTISSDNDKNTVYLQMNSLKPEDTAVYYCAAVPAGRLRFGEQWYPIYEYDAWGQGTLVTVSS

SEQ ID NO: 90 - protein, 129 aa, Artificial
  Mutated Lama sequence
EVQLVESGGGLVQAGGSLRLSCAASGITLDDYAIGWFRQAPGKERVGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 91 - protein, 129 aa, Artificial
  Mutated Lama sequence
EVQLVESGGGLVQAGGSLRLSCAASGLTLDDYAIGWFRQAPGKEREGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 92 - protein, 129 aa, Artificial
  Mutated Lama sequence
QVQLVESGGGLVQAGGSLRLSCAVSGFTLDDYAIGWFRQAPGKEREGVSSIRDNDGSTYYADSVKGRFTISSDNDQNTVYLQMNSLKSEDTAVYYCAAVPAGRLRFGEQWYPIYEYDAWGQGTLVTVSS

SEQ ID NO: 93 - protein, 129 aa, Artificial
  Mutated Lama sequence
EVQLVESGGGLVQAGGSLRLSCAISGFTLVDYAIGWFRQAPGKEREGVSSIRDNDGSTYYADSVKGRFIISSDNDKNTVYLQMNSLKPEDTAVYYCAAVPAGRLRFGEQWYPIYEYDAWGQGTLVTVSS

SEQ ID NO: 94 - protein, 129 aa, Artificial
  Mutated Lama sequence
EVQLMESGGGLVQAGGSLRLSCAASGITLDDYAIGWFRQAPGKEREGVSSIRDNDGSTYYADSVKGRFTISSDNDKNTVNLQMNSLKPEDTAVYYCAAVPAGRLRFGEQWYPIYEYDAWGQGTLVTVSS

SEQ ID NO: 95 - protein, 129 aa, Artificial
  Mutated Lama sequence
EVQLVESGGGLVQAGGSLRLSCAASGFTLDDYAIGWFRQAPGKEREGVSSIRDNDGSTYYADSVKGRFTISSDNDKNTVYLQMNSLKPEDTAVYYCAAVPAGRLRYGEQWYPIYEYDAWGQGTLVTVSS

SEQ ID NO: 96 - protein, 129 aa, Artificial
  Mutated Lama sequence
EVQLVESGGGLVQAGGSLRLSCAVSGFTLDDYAIGWFRQAPGKEREGVSSIRENDGSTYYADSVQGRFTISSDNDKNTVYLRMNSLKPEDTAVYYCAAVPAGRLRFGEQWYPIYEYDAWGQGTLVTVSS

SEQ ID NO: 97 - protein, 129 aa, Artificial
  Mutated Lama sequence
EVQMVESGGGLVQAGGSLRLSCAASGLTLDDYAIGWFRQAPGKEREGVSSIRDNDGSTYYADSVKGRFTISSDNDKNTVYLQMNGLKPEDTAVYYCAAVPAGRLRFGEQWYPIYEYDAWGQGTLVTVSS

SEQ ID NO: 98 - protein, 129 aa, Artificial
  Mutated Lama sequence
EVQLVESGGGLVQSGGSLRLSCAASGLTLDDYAIGWFRQAPGKEREGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 99 - protein, 129 aa, Artificial
  Mutated Lama sequence
EVQLVESGGGLVQAGGSLRLSCAVSGFTLDDYAIGWFRQAPGKEREGVSSIRDNDGSTYYADSVKGRSTISSDNDKNTVYLQMNSLKPEDTAVYYCAAVPAGRLRFGEQWYPIYEYDAWGQGTLVTVSS

SEQ ID NO: 100 - protein, 129 aa, Artificial
  Mutated Lama sequence
EVQLVESGGGLVQAGGSLRLSCAVSGITLDDYAIGWFRQAPGKEREGVSSIRDNDGSTYYADSVKWRFTISSDNDKNTVYLQMNSMKPEDTAVYYCAAVPAGRLRFGEQWYPIYEYDAWGQGILVTVSS

SEQ ID NO: 101 - protein, 129 aa, Artificial
  Mutated Lama sequence
EVQLVESGGGLVQAGGFLRLTCAVSGFTLDDYAIGWFRQAPGKEREGVSSIRDNDGSTYYADSVKGRFTISSDNVKNTVYLQMNSLKPEDTAVYYCAAVPAGRLRFGEQWYPIYEYDAWGQGTLVTVSS

SEQ ID NO: 102 - protein, 129 aa, Artificial
  Mutated Lama sequence
EVQLLESGGGLVQAGGSLRLSCAASGFTLDDYAIGWFRQAPGKEREGVSSIRDNDGSTYYADSVKGRFTISSDNDKNTVYLQMNSLKPEDTAVYYCAAVPAGRLRIGEQWYPIYEYDAWGQGTLVTVSS

SEQ ID NO: 103 - protein, 129 aa, Artificial
  Mutated Lama sequence
EVHLVESGGGLVQAGGSLRLSCAASGLTLDDYAIGWFRQAPGVEREGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 104 - protein, 129 aa, Artificial
  Mutated Lama sequence
EVQLVESGGGLVQAGGSLRLSCAASGFTLDDYAIGWFRQAPGKDREGVSSIRDNDGSTYYADSVKGRFTISSDNDKNTVYLQMNSLKPEDTAVYYCAAVPAGRLRYGEQWYPIYEYDAWGQGTVVTVSS

SEQ ID NO: 105 - protein, 129 aa, Artificial
  Mutated Lama sequence
EVQLVESGGGLVQAGGTLRLSCAVSGFTLDDYAIGWFRQAPGKEREGVSSIRDNDGSTYYADSVKGRFTISSDNDKNTVYLQMNSLKPEDTAVYYCAAVPAGRLRFGEQWYPIYEYDAWGQGILVTVSS

SEQ ID NO: 106 - protein, 129 aa, Artificial
  Mutated Lama sequence
EVQLVESGGGLVQAGGSLRLSCAASGFTLDDYAIGWFRQAPGKEREGVSSIRDNDGSTYYADSVKGRFIISSDNDKNTVYLQMNNLKPEDTAVYYCAAVPAGRLRYGEQWYPIYEYDAWGQGTLVTVSS

SEQ ID NO: 107 - protein, 129 aa, Artificial
  Mutated Lama sequence
EVQLVESGGGLVQAGGSLRLSCAASGFTLDDYAIGWFRQAPGKEREGVSSIRDNDGSTYYADSVKGRSTISSDNARNTVFLQMNSLKPEDTAVYYCAAVPAGRLRFGEQWYPIYEYDAWGQGTLVTVSS

SEQ ID NO: 108 - protein, 129 aa, Artificial
  Mutated Lama sequence
EELLVESGGGSVQAGGSLKLSCAASGLTLDDYAIGWFRQAPGKEREGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 109 - protein, 129 aa, Artificial
  Mutated Lama sequence
EVQLVESGGGLVQAGGSLRLSCAASGLTLDDYAIGWFRQAPGKEREGVSSIRDNDGSRYYADSVKGRFTISSDNDKNTVYLQMNSLKPEDTAVYYCAAVPAGRLRFGEQWYPIYEYDAWGQGTLVTVSS

SEQ ID NO: 110 - protein, 129 aa, Artificial
  Mutated Lama sequence
EVQLVESGGSLVQAGGSLRLSCAASGLTLDDYAIGWFRQAPGKEREGVSSIRDNDGSTYYADSVKGRFTISSDNDKNTVYLQMNSLKPEDTAVYYCAAVPAGRLRFGEQWYPIYEYDAWGQGTLVTVSS

SEQ ID NO: 111 - protein, 129 aa, Artificial; identical to SEQ ID NO: 110
  Mutated Lama sequence

SEQ ID NO: 112 - protein, 129 aa, Artificial
  Mutated Lama sequence
EVQLVESGGGLVQPGGSLRLSCAASGFTLDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 113 - protein, 129 aa, Artificial
  Mutated Lama sequence
EVQLVESGGGLVQPGGSLRLSCAASGFTLDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 114 - protein, 129 aa, Artificial
  Mutated Lama sequence
EVQLVESGGGLVQPGGSLRLSCAASGFTIDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 115 - protein, 129 aa, Artificial
  Mutated Lama sequence
EVQLVESGGGLVQPGGSLRLSCAASGFTIDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 116 - protein, 129 aa, Artificial
  Mutated Lama sequence
EVQLVESGGGLVQPGGSLRLSCAASGITLDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 117 - protein, 129 aa, Artificial
  Mutated Lama sequence
EVQLVESGGGLVQPGGSLRLSCAASGITLDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 118 - protein, 129 aa, Artificial
  Mutated Lama sequence
EVQLVESGGGLVQPGGSLRLSCAASGITIDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 119 - protein, 129 aa, Artificial
  Mutated Lama sequence
EVQLVESGGGLVQPGGSLRLSCAASGITIDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 120 - protein, 129 aa, Artificial
  Mutated Lama sequence
EVQLVESGGGLVQPGGSLRLSCAASGLTLDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 121 - protein, 129 aa, Artificial
  Mutated Lama sequence
EVQLVESGGGLVQPGGSLRLSCAASGLTLDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 122 - protein, 129 aa, Artificial
  Mutated Lama sequence
EVQLVESGGGLVQPGGSLRLSCAASGLTIDDYAIGWFRQAPGKEREGVSSIRDNGGSTYYADSVKGRFTISSDNSKNTVYLQMNSLRPEDTAVYYCAAVPAGRLRFGEQWYPIYEYDAWGQGTLVTVSS

SEQ ID NO: 123 - protein, 129 aa, Artificial
  Mutated Lama sequence
EVQLVESGGGLVQPGGSLRLSCAASGLTIDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 124 - protein, 124 aa, Artificial
  Mutated Lama sequence
EVQLVESGGGLVQPGGSLRLSCAASGFTFDDYALGWFRQAAGKEREGVSCIRCSDGSTYYADSVKGRFTISRDNSKNTVYLQMNSLRPEDTAVYYCAASIVPRSKLEPYEYDAWGQGTLVTVSS

SEQ ID NO: 125 - protein, 124 aa, Artificial
  Mutated Lama sequence
EVQLVESGGGLVQPGGSLRLSCAASGFTFDDYALGWFRQAPGKEREGVSCIRCSDGSTYYADSVKGRFTISRDNSKNTVYLQMNSLRPEDTAVYYCAASIVPRSKLEPYEYDAWGQGTLVTVSS

SEQ ID NO: 126 - protein, 124 aa, Artificial
  Mutated Lama sequence
EVQLVESGGGLVQPGGSLRLSCAASGFTFDDYALGWFRQAAGKEREGVSCIRCSDGSTYYADSVKGRFTISSDNSKNTVYLQMNSLRPEDTAVYYCAASIVPRSKLEPYEYDAWGQGTLVTVSS

SEQ ID NO: 127 - protein, 124 aa, Artificial
  Mutated Lama sequence
EVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSS

SEQ ID NO: 128 - protein, 124 aa, Artificial
  Mutated Lama sequence
EVQLVESGGGLVQPGGSLRLSCAASGFTFDDYALGWFRQAAGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 129 - protein, 124 aa, Artificial
  Mutated Lama sequence
EVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 130 - protein, 124 aa, Artificial
  Mutated Lama sequence
DVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 131 - protein, 126 aa, Artificial
  Mutated Lama sequence
EVQLVESGGGLVQPGGSLRLSCAASGFALDYYAIGWFRQAPGKEREGVSCISSSDGITYYVDSVKGRFTISRDNSKNTVYLQMNSLRPEDTAVYYCATDSGGYIDYDCMGLGYDYWGQGTLVTVSS

SEQ ID NO: 132 - protein, 126 aa, Artificial
  Mutated Lama sequence
EVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSS

SEQ ID NO: 133 - protein, 126 aa, Artificial
  Mutated Lama sequence
EVQLVESGGGLVQPGGSLRLSCAASGFALDYYAIGWFRQAPGKEREGVSCISSSGGITYYVDSVKGRFTISRDNSKNTVYLQMNSLRPEDTAVYYCATDSGGYIDYDCMGLGYDYWGQGTLVTVSS

SEQ ID NO: 134 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCMGLGYDYWGQGTLVTVSS

SEQ ID NO: 135 - protein, 126 aa, Artificial
  Mutated Lama sequence
DVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCMGLGYDYWGQGTLVTVSS

SEQ ID NO: 136 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCQGLGYDYWGQGTLVTVSS

SEQ ID NO: 137 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCRGLGYDYWGQGTLVTVSS

SEQ ID NO: 138 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSS

SEQ ID NO: 139 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATESGGYIDYDCQGLGYDYWGQGTLVTVSS

SEQ ID NO: 140 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATESGGYIDYDCRGLGYDYWGQGTLVTVSS

SEQ ID NO: 141 - protein, 126 aa, Artificial
  Mutated Lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATESGGYIDYDCSGLGYDYWGQGTLVTVSS

SEQ ID NO: 142 - protein, 129 aa, Artificial
  Mutated Lama sequence
EVQLVESGGGLVQAGGSLRLSCAASGFTLDDYAIGWFRQAPGKEREGVSTIRDSDGSTYYADSVKGRFTISSDNDKNTVYLQMNSLKPEDTAVYYCAAVPAGRLRFGEQWYPLYEYDAWGQGTLVTVSS

SEQ ID NO: 143 - protein, 129 aa, Artificial
  Mutated Lama sequence
EVQLVESGGGLVQAGGSLRLSCAASGFTLDDYAIGWFRQAPGKEREGVSAIRDSDGSTYYADSVKGRFTISSDNDKNTVYLQMNSLKPEDTAVYYCAAVPAGRLRFGEQWYPLYEYDAWGQGTLVTVSS

SEQ ID NO: 144 - protein, 129 aa, Artificial
  Mutated Lama sequence
EVQLVESGGGLVQAGGSLRLSCAASGFTLDDYAIGWFRQAPGKEREGVSSIRDNDGSTYYADSVKGRFTISSDNDKNTVYLQMNSLKPEDTAVYYCAAVPAGRLRFGEQWYPLYEYDAWGQGTLVTVSS

SEQ ID NO: 145 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISSDNAKNTVYLQMNSLRPEDTAVYYCAAVPAGRLRFGEQWYPLYEYDAWGQGTLVTVSS

SEQ ID NO: 146 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSS

SEQ ID NO: 147 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISRDNAKNTVYLQMNSLRPEDTAVYYCAAVPAGRLRFGEQWYPLYEYDAWGQGTLVTVSS

SEQ ID NO: 148 - protein, 129 aa, Artificial
  Mutated Lama sequence
EVQLVESGGGLVQAGGSLRLSCAASGFTLDDYAIGWFRQAPGKEREGVSSIRDSDGSTYYADSVKGRFTISSDNDKNTVYLQMNSLKPEDTAVYYCAAVPAGRLRFGEQWYPLYEYDAWGQGTLVTVSS

SEQ ID NO: 149 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISSDNDKNTVYLQMNSLRPEDTAVYYCAAVPAGRLRFGEQWYPLYEYDAWGQGTLVTVSS

SEQ ID NO: 150 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISRDNDKNTVYLQMNSLRPEDTAVYYCAAVPAGRLRFGEQWYPLYEYDAWGQGTLVTVSS

SEQ ID NO: 151 - protein, 129 aa, Artificial
  Mutated Lama sequence
EVQLVESGGGLVQPGGSLRLSCAASGFTLDDYAIGWFRQAPGKEREGVSSIRDNDGSTYYADSVKGRFTISRDNSKNTVYLQMNSLRPEDTAVYYCAAVPAGRLRFGEQWYPLYEYDAWGQGTLVTVSS

SEQ ID NO: 152 - protein, 129 aa, Artificial
  Mutated Lama sequence
EVQLVESGGGLVQAGGSLRLSCAASGFTLDDYAIGWFRQAPGKEREGVSSIRDNGGSTYYADSVKGRFTISSDNDKNTVYLQMNSLKPEDTAVYYCAAVPAGRLRFGEQWYPLYEYDAWGQGTLVTVSS

SEQ ID NO: 153 - protein, 129 aa, Artificial
  Mutated Lama sequence
EVQLVESGGGLVQAGGSLRLSCAASGFTLDDYAIGWFRQAPGKEREGVSAIRDNDGSTYYADSVKGRFTISSDNDKNTVYLQMNSLKPEDTAVYYCAAVPAGRLRFGEQWYPLYEYDAWGQGTLVTVSS

SEQ ID NO: 154 - protein, 129 aa, Artificial
  Mutated Lama sequence
EVQLVESGGGLVQAGGSLRLSCAASGFTLDDYAIGWFRQAPGKEREGVSSIRDIDGSTYYADSVKGRFTISSDNDKNTVYLQMNSLKPEDTAVYYCAAVPAGRLRFGEQWYPLYEYDAWGQGTLVTVSS

SEQ ID NO: 155 - protein, 129 aa, Artificial
  Mutated Lama sequence
EVQLVESGGGLVQAGGSLRLSCAVSGFTLDDYAIGWFRQAPGKEREGVSSIRDNDGSTYYADSVKGRFTISSDNDKNTVYLQMNSLKPEDTAVYYCAAVPAGRLRFGEQWYPLYEYDAWGQGTLVTVSS

SEQ ID NO: 156 - protein, 129 aa, Artificial; identical to SEQ ID NO: 143
  Mutated Lama sequence

SEQ ID NO: 157 - protein, 129 aa, Artificial
  Mutated Lama sequence
EVQLVESGGGLVQAGGSLRLSCAASGFTLDDYAIGWFRQAPGKEREGVSAIRDSGGSTYYADSVKGRFTISSDNDKNTVYLQMNSLKPEDTAVYYCAAVPAGRLRFGEQWYPLYEYDAWGQGTLVTVSS

SEQ ID NO: 158 - protein, 129 aa, Artificial
  Mutated Lama sequence
EVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSS

SEQ ID NO: 159 - protein, 129 aa, Artificial
  Mutated Lama sequence
DVQLVESGGGLVQPGGSLRLSCAASGFTLDDYAIGWFRQAPGKEREGVSAIRDNGGSTYYADSVKGRFTISSDNSKNTVYLQMNSLRPEDTAVYYCAAVPAGRLRFGEQWYPLYEYDAWGQGTLVTVSS

SEQ ID NO: 160 - protein, 129 aa, Artificial
  Mutated Lama sequence
EVQLVESGGGLVQPGGSLRLSCAVSGITLDDYAIGWFRQAPGKEREGVSSIRDNGGSTYYADSVKGRFTISSDNSKNTVYLQMNSLRPEDTAVYYCAAVPAGRLRYGEQWYPIYEYDAWGQGTLVTVSS

SEQ ID NO: 161 - protein, 129 aa, Artificial
  Mutated Lama sequence
EVQLVESGGGLVQPGGSLRLSCAASGITLDDYAIGWFRQAPGKEREGVSAIRDNGGSTYYADSVKGRFTISSDNSKNTVYLQMNSLRPEDTAVYYCAAVPAGRLRYGEQWYPIYEYDAWGQGTLVTVSS

SEQ ID NO: 162 - protein, 129 aa, Artificial
  Mutated Lama sequence
EVQLVESGGGLVQPGGSLRLSCAASGITLDDYAIGWFRQAPGKEREGVSAIRESGGSTYYADSVKGRFTISSDNSKNTVYLQMNSLRPEDTAVYYCAAVPAGRLRYGEQWYPIYEYDAWGQGTLVTVSS

SEQ ID NO: 163 - protein, 129 aa, Artificial
  Mutated Lama sequence
EVQLVESGGGLVQPGGSLRLSCAASGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSS

SEQ ID NO: 164 - protein, 129 aa, Artificial
  Mutated Lama sequence
EVQLVESGGGLVQPGGSLRLSCAVSGITLDDYAIGWFRQAPGKEREGVSAIRDNGGSTYYADSVKGRFTISSDNSKNTVYLQMNSLRPEDTAVYYCAAVPAGRLRYGEQWYPIYEYDAWGQGTLVTVSS

SEQ ID NO: 165 - protein, 129 aa, Artificial
  Mutated Lama sequence
EVQLVESGGGLVQPGGSLRLSCAVSGITLDDYAIGWFRQAPGKEREGVSAIRESGGSTYYADSVKGRFTISSDNSKNTVYLQMNSLRPEDTAVYYCAAVPAGRLRYGEQWYPIYEYDAWGQGTLVTVSS

SEQ ID NO: 166 - protein, 129 aa, Artificial
  Mutated Lama sequence
EVQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSS

SEQ ID NO: 167 - protein, 129 aa, Artificial
  Mutated Lama sequence
EVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSS

SEQ ID NO: 168 - protein, 5 aa, Artificial
  Mutated Lama sequence
DYAIG

SEQ ID NO: 169 - protein, 5 aa, Artificial
  Mutated Lama sequence
DYALG

SEQ ID NO: 170 - protein, 5 aa, Artificial
  Mutated Lama sequence
YYAIG

SEQ ID NO: 171 - protein, 17 aa, Artificial
  Mutated Lama sequence
AIRSSGGSTYYADSVKG

SEQ ID NO: 172 - protein, 17 aa, Artificial
  Mutated Lama sequence
CIRCSGGSTYYADSVKG

SEQ ID NO: 173 - protein, 17 aa, Artificial
  Mutated Lama sequence
CISSSGGITYYADSVKG

SEQ ID NO: 174 - protein, 20 aa, Artificial
  Mutated Lama sequence
VPAGRLRFGEQWYPLYEYDA

SEQ ID NO: 175 - protein, 20 aa, Artificial
  Mutated Lama sequence
VPAGRLRYGEQWYPIYEYDA

SEQ ID NO: 176 - protein, 15 aa, Artificial
  Mutated Lama sequence
SIVPRSKLEPYEYDA

SEQ ID NO: 177 - protein, 17 aa, Artificial
  Mutated Lama sequence
DSGGYIDYDCSGLGYDY

SEQ ID NO: 178 - DNA, 387 bp, Artificial
  Mutated Lama sequence
gaggtacagctggtcgagtcaggtggcggattagtgcagcctgggggttctctgcgcttatcttgtgccgcatcaggcttcacactggatgactacgccatcggctggttccggcaagcgcctggaaaagaacgcgaaggtgtttcagcaatccgttcaagcggtggttcaacatattacgccgactctgttaaaggacgcttcaccattagctccgacaatagtaaaaatacagtctacttacaaatgaacagtttacgcccagaagatactgcggtatactattgcgctgccgtgcctgctggtcgcttacgctttggcgagcaatggtatcctctgtacgagtacgacgcctggggacagggtacgctggtaacggtttcaagc

SEQ ID NO: 179 - DNA, 387 bp, Artificial
  Mutated Lama sequence
gaagtgcaactggttgagtcaggtggcggactggtgcaaccgggtggttcactgcgcctgagttgcgcagttagcggtattaccctggatgattatgcaattggttggtttcgccaagccccaggcaaagagcgtgaaggcgttagcgcaattcgtagcagcggtggtagcacctattatgccgattcagttaaaggccgttttacgatcagcagcgataacagtaaaaacacggtttatctgcaaatgaactcattacgtccagaggacactgcagtttactattgcgcagcagttccggcaggtcgtctgcgttatggtgaacagtggtatccgatttatgaatatgatgcatggggtcaaggtacactggttacagtgagtagc

SEQ ID NO: 180 - DNA, 372 bp, Artificial
  Mutated Lama sequence
gaagtgcagttagtcgaaagtggcggaggcctggtacaacctggtggcagtctgcgcttatcttgtgccgcttcaggttttacattcgacgactacgccctggggtggttccggcaagcgcctggaaaagaacgtgagggcgtttcatgcattcgttgttcaggtggttcaacctattatgccgatagtgtaaaaggtcggttcaccattagtagcgacaatagcaagaatacagtctatctgcaaatgaactctttacgtcctgaagatactgcggtgtactactgcgctgcatcaatcgttcctcgttcaaaacttgaaccttacgagtacgacgcctggggtcagggtacgttagtaacggtgtcaagc

SEQ ID NO: 181 - DNA, 378 bp, Artificial
  Mutated Lama sequence
gaagtgcaactggttgagtcaggtggcggtttagtgcaaccgggtggttcactgcgcctgagttgcgcagccagcggttttgcactggattattatgcaattggttggtttcgccaagccccaggcaaagagcgtgaaggcgttagctgtattagcagcagcggtggtattacctattatgccgattcagttaaaggccgttttacgatctctcgtgataatagtaaaaacacggtttacctgcagatgaactcattaagaccagaggacactgcagtttactattgtgcaaccgatagcggtggctatattgattatgattgtagcggtctgggctacgattattggggacaaggtacgctggtgacagttagcagc

SEQ ID NO: 182 - DNA, 3375 bp, Homo sapiens
atggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcggctgtgatttcttggacaatattggatggctattctatttcttctattactatccgttacaaggttcaaggcaagaatgaagaccagcacgttgatgtgaagataaagaatgccaccatcattcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtacgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctag

SEQ ID NO: 183 - DNA, 4654 bp, Mouse
gagcaggagccggagcaggagcagaagataagccttggatgaagggcaagatggatagggctcgctctgccccaagccctgctgataccaagtgcctttaagatacagcctttcccatcctaatctgcaaaggaaacaggaaaaaggaacttaaccctccctgtgctcagacagaaatgagactgttaccgcctgcttctgtggtgtttctccttgccgccaacttgtaaacaagagcgagtggaccatgcgagcgggaagtcgcaaagttgtgagttgttgaaagcttcccagggactcatgctcatctgtggacgctggatggggagatctggggaagtatggactctttagccggcttagttctctgtggagtcagcttgctcctttatggagtagtagaaggcgccatggacctgatcttgatcaattccctacctcttgtgtctgatgccgaaacatccctcacctgcattgcctctgggtggcacccccatgagcccatcaccataggaagggactttgaagccttaatgaaccagcaccaagatccactggaggttactcaagatgtgaccagagaatgggcgaaaaaagttgtttggaagagagaaaaggccagtaagattaatggtgcttatttctgtgaaggtcgagttcgaggacaggctataaggatacggaccatgaagatgcgtcaacaagcatccttcctacctgctactttaactatgaccgtggacaggggagataatgtgaacatatctttcaaaaaggtgttaattaaagaagaagatgcagtgatttacaaaaatggctccttcatccactcagtgccccggcatgaagtacctgatattttagaagttcacttgccgcatgctcagccccaggatgctggtgtgtactcggccaggtacataggaggaaacctgttcacctcagccttcaccaggctgattgttcggagatgtgaagctcagaagtgggggcccgactgtagccgtccttgtactacttgcaagaacaatggagtctgccatgaagataccggggaatgcatttgccctcctgggtttatggggagaacatgtgagaaagcttgtgagccgcacacatttggcaggacctgtaaagaaaggtgtagtggaccagaaggatgcaagtcttatgtgttctgtctcccagacccttacgggtgttcctgtgccacaggctggagggggttgcagtgcaatgaagcatgcccatctggttactacggaccagactgtaagctcaggtgccactgtaccaatgaagagatatgtgatcggttccaaggatgcctctgctctcaaggatggcaagggctgcagtgtgagaaagaaggcaggccaaggatgactccacagatagaggatttgccagatcacattgaagtaaacagtggaaaatttaaccccatctgcaaagcctctgggtggccactacctactagtgaagaaatgaccctagtgaagccagatgggacagtgctccaaccaaatgacttcaactatacagatcgtttctcagtggccatattcactgtcaaccgagtcttacctcctgactcaggagtctgggtctgcagtgtgaacacagtggctgggatggtggaaaagcctttcaacatttccgtcaaagttcttccagagcccctgcacgccccaaatgtgattgacactggacataactttgctatcatcaatatcagctctgagccttactttggggatggacccatcaaatccaagaagcttttctataaacctgtcaatcaggcctggaaatacattgaagtgacgaatgagattttcactctcaactacttggagccgcggactgactacgagctgtgtgtgcagctggcccgtcctggagagggtggagaagggcatcctgggcctgtgagacgatttacaacagcgtctatcggactccctcctccaagaggtctcagtctcctgccaaaaagccagacagctctaaatttgacttggcaaccgatatttacaaactcagaagatgaattttatgtggaagtcgagaggcgatccctgcaaacaacaagtgatcagcagaacatcaaagtgcctgggaacctgacctcggtgctactgagcaacttagtccccagggagcagtacacagtccgagctagagtcaacaccaaggcgcagggggagtggagtgaagaactcagggcctggacccttagtgacattctccctcctcaaccagaaaacatcaagatctccaacatcactgactccacagctatggtttcttggacaatagtggatggctattcgatttcttccatcatcatccggtataaggttcagggcaaaaatgaagaccagcacattgatgtgaagatcaagaatgctaccgttactcagtaccagctcaagggcctagagccagagactacataccatgtggatatttttgctgagaacaacataggatcaagcaacccagccttttctcatgaactgaggacgcttccacattccccagcctctgcagacctcggagggggaaagatgctactcatagccatccttgggtcggctggaatgacttgcatcaccgtgctgttggcgtttctgattatgttgcaactgaagagagcaaatgtccaaaggagaatggctcaggcattccagaacgtgagagaagaaccagctgtgcagtttaactcaggaactctggcccttaacaggaaggccaaaaacaatccggatcccacaatttatcctgtgcttgactggaatgacatcaagtttcaagacgtgatcggagagggcaactttggccaggttctgaaggcacgcatcaagaaggatgggttacggatggatgccgccatcaagaggatgaaagagtatgcctccaaagatgatcacagggacttcgcaggagaactggaggttctttgtaaacttggacaccatccaaacatcattaatctcttgggagcatgtgaacaccgaggctatttgtacctagctattgagtatgccccgcatggaaacctcctggacttcctgcgtaagagcagagtgctagagacagaccctgcttttgccatcgccaacagtacagcttccacactgtcctcccaacagcttcttcattttgctgcagatgtggcccgggggatggactacttgagccagaaacagtttatccacagggacctggctgccagaaacattttagttggtgaaaactacatagccaaaatagcagattttggattgtcacgaggtcaagaagtgtatgtgaaaaagacaatgggaaggctcccagtgcgttggatggcaatcgaatcactgaactatagtgtctatacaaccaacagtgatgtctggtcctatggtgtattgctctgggagattgttagcttaggaggcaccccctactgcggcatgacgtgcgcggagctctatgagaagctaccccagggctacaggctggagaagcccctgaactgtgatgatgaggtgtatgatctaatgagacagtgctggagggagaagccttatgagagaccatcatttgcccagatattggtgtccttaaacaggatgctggaagaacggaagacatacgtgaacaccacactgtatgagaagtttacctatgcaggaattgactgctctgcggaagaagcagcctagagcagaactcttcatgtacaacggccatttctcctcactggcgcgagagcgccttgacacctgtaccaagcaagccacccactgccaagagatgtgatatataagtgtatatattgtgctgtgtttgggaccctcctcatacagctcgtgcggatctgcagtgtgttctgactctaatgtgactgtatatactgctcggagtaagaatgtgctaagatcagaatgcctgttcgtggtttcatataatatatttttctaaaagcatagattgcacaggaaggtatgagtacaaatactgtaatgcataacttgttattgtcctagatgtgtttgatatttttcctttacaactgaatgctataaaagtgttttgctgtgtacacataagatactgttcgttaaaataagcattcccttgacagcacaggaagaaaagcgagggaaatgtatggattatattaaatgtgggttactacacaagaggccgaacattccaagtagcagaagagagggtctctcaactctgctcctcacctgcagaagccagtttgtttggccatgtgacaattgtcctgtgtttttatagcacccaaatcattctaaaatatgaacatctaaaaactttgctaggagactaagaacctttggagagatagatataagtacggtcaaaaaacaaaactgtgggacttacatttattttctatagtaatctgttgtacattttaagaagtaaaactaggaatttaggagtgatgtgtgacatttctgacatggagttaccatccccacatgtatcacatactgtcatattcccacatgtatcacacatgtattgtaaaattttgtagttttgatcacttgtgaatttactgttgatgtggtagccacctgctgcaatggttcctcttgtaggtgaataaatgtcttgtctacccaca

SEQ ID NO: 184 - DNA, 3378 bp, Artificial
  Mutated Cyno sequence
atggacagcctggccagcctggtgctgtgcggagtgtccctgctgctgagcggcacagtggaaggcgccatggacctgatcctgatcaactccctgcccctggtgtccgacgccgagacaagcctgacctgtatcgctagcggctggcacccccacgagcctatcaccatcggccgggacttcgaggccctgatgaaccagcaccaggaccccctggaagtgacccaggacgtgacccgcgagtgggccaagaaagtcgtgtggaagagagagaaggccagcaagatcaacggcgcctacttctgcgagggcagagtgcggggcgaggccatcagaatccggacaatgaagatgcggcagcaggccagcttcctgcccgccaccctgaccatgaccgtggacaagggcgacaacgtgaacatcagcttcaagaaggtgctgatcaaagaagaggacgccgtcatctacaagaacggcagcttcatccacagcgtgccccggcacgaggtgcccgacatcctggaagtgcatctgccccacgcccagcctcaggatgccggcgtgtacagcgcccggtacatcggcggcaacctgttcaccagcgccttcacccggctgatcgtgcggagatgcgaggcccagaaatggggccctgagtgtaaccggctgtgcaccgtgtgcgtgaacaacggcgtgtgccacgaggacaccggcgagtgcatctgtcctcccggcttcatgggccggacctgcgagaaggcctgcgagagacacaccttcggcagaacctgcaaagagcggtgtagcggccaggacggctgcaagagctacgtgttctgcctgcccgacccctacggctgcagctgtgccacaggctggaagggcctgcagtgcaacgaggcctgccaccacggcttctacggccccgactgcaagctgcggtgcagctgcagcaacggcgagacatgcgaccggttccagggatgcctgtgcagccctggcagacagggactccagtgcgagcgcgagggcatccccagaatgacccccaagatcgtggacctgcccgatcacatcgaagtgaacagcggcaagttcaaccccatctgcaaggcctccggctggcccctgcccaccaacgaagagatgaccctggtcaagcccgacggcaccgtgctgcaccccaaggacttcaaccacaccgaccacttcagcgtggccatcttcaccatccaccggatcctgccccccgactctggcgtgtgggtctgcagcgccaataccgtggccggcatggtggaaaagcccttcaacatcagcgtgaaggtgctgcccaagcccctgaacgcccccaacgtgatcgacaccggccacaacttcgccgtgatcaacatctccagcgagccctacttcggcgacggccccatcaagagcaagaagctgctgtacaagcccgtgaaccactacgaggcctggcggcacatccaggtcacaaacgagatcgtgaccctgaaccacctggaaccccggaccgagtacgagctgtgcgtgcagctcgtgcggaggggcgaaggcggcgaaggacatcctggccccgtgcgcagattcaccaccgcctctatcggcctgcccccacccagaggcctgaacctgctgcctaagagccagaccaccctgaacctgacctggcagcccatcttccccagcagcgaggacgacttctacgtggaagtggaacggcggagcgtgcagaagtccgaccagcagaacatcaaggtgcccggcaacctgaccagcgtgctgctgaacaacctgcacccccgcgagcagtacgtcgtgcgggccagagtgaacaccaaggcccagggcgagtggtccgaggatctgaccgcctggaccctgagcgacatcctgcctccccagcccgagaatatcaaaatcagcaacatcacccacagcagcgccgtgatctcttggaccatcctggacggctacagcatcagctccatcaccatccggtacaaggtgcagggcaagaacgaggaccagcacatcgacgtgaagatcaagaacgccaccatcacccagtaccagctgaagggcctggaacccgagacagcctaccaggtggacatcttcgccgagaacaatatcggcagcagcaaccccgccttcagccacgagctggtcaccctgcccgagtctgaggcccctgctgatctgggcggaggcaagatgctgctgatcgccatcctgggcagcgccggcatgacctgtctgacagtgctgctggccttcctgatcatcctgcagctgaagcgggccaacgtgcagcggagaatggcccaggccttccagaacgtgcgcgaggaacccgccgtgcagttcaacagcggcaccctggccctgaaccggaaagtgaagaacaaccccgaccccaccatctaccccgtgctggactggaacgacatcaagttccaggatgtgatcggcgagggcaacttcggccaggtgctgaaggcccggatcaagaaagacggcctgcggatggacgccgccatcaagcggatgaaggaatacgccagcaaggacgaccacagagacttcgccggggagctggaagtgctgtgcaagctgggccaccaccccaacatcatcaacctgctgggcgcctgcgagcaccggggctatctgtacctggccatcgagtacgccccccacggcaatctgctggatttcctgcggaagtcccgggtgctggaaaccgaccctgccttcgccattgccaacagcaccgccagcaccctgagcagccagcagctcctgcacttcgccgccgatgtggccagaggcatggactacctgagccagaagcagttcatccaccgggacctggccgccagaaacatcctcgtgggcgagaactacgtggccaagatcgccgacttcggcctgagcagaggccaggaagtgtacgtcaagaaaaccatgggccggctgcccgtgcggtggatggccattgagagcctgaactacagcgtgtacaccaccaacagcgacgtgtggtcctacggggtgctgctgtgggagatcgtgtctctgggcggcaccccctactgcggcatgacatgcgccgagctgtacgagaagctgccccagggctaccggctggaaaagcctctgaactgcgacgacgaggtgttcgacctgatgcggcagtgctggcgcgagaagccctacgagcggcctagcttcgcccagatcctggtgtccctgaacagaatgctggaagaacgcaagacctacgtgaacaccaccctgtatgagaagttcacctacgccggcatcgactgcagcgccgaagaggccgcttgatga

SEQ ID NO: 185 - DNA, 1491 bp, Mouse
atgtggcagatcattttcctaacttttggctgggatcttgtcttggcctcagcctacagtaactttaggaagagcgtggacagcacaggcagaaggcagtaccaggtccagaacggaccctgcagctacacgttcctgctgccggagaccgacagctgccgatcttcctccagcccctacatgtccaatgccgtgcagagggatgcacccctcgactacgacgactcagtgcaaaggctgcaggtgctggagaacattctagagaacaacacacagtggctgatgaagctggagaattacattcaggacaacatgaagaaggagatggtggagatccaacagaatgtggtgcagaaccagacagctgtgatgatagagattggaaccagcttgctgaaccagacagcagcacaaactcggaaactgactgatgtggaagcccaagtactaaaccagacgacaagactcgagctgcagcttctccaacattctatttctaccaacaaattggaaaagcagattttggatcagaccagtgaaataaacaagctacaaaataagaacagcttcctagaacagaaagttctggacatggagggcaagcacagcgagcagctacagtccatgaaggagcagaaggacgagctccaggtgctggtgtccaagcagagctctgtcattgacgagctggagaagaagctggtgacagccacggtcaacaactcgctccttcagaagcagcagcatgacctaatggagaccgtcaacagcttgctgaccatgatgtcatcacccaactccaagagctcggttgctatccgtaaagaagagcaaaccaccttcagagactgtgcggaaatcttcaagtcaggactcaccaccagtggcatctacacactgaccttccccaactccacagaggagatcaaggcctactgtgacatggacgtgggtggaggagggtggacagtcatccaacaccgagaagatggcagtgtggacttccagaggacgtggaaagaatacaaagagggcttcgggagccctctgggagagtactggctgggcaatgagtttgtctcccagctgaccggtcagcaccgctacgtgcttaagatccagctgaaggactgggaaggcaacgaggcgcattcgctgtatgatcacttctacctcgctggtgaagagtccaactacaggattcaccttacaggactcacggggaccgcgggcaaaataagtagcatcagccaaccaggaagtgattttagcacaaaggattcggacaatgacaaatgcatctgcaagtgttcccagatgctctcaggaggctggtggtttgacgcatgtggtccttccaacttgaatggacagtactacccacaaaaacagaatacaaataagtttaacggtatcaagtggtactactggaaggggtccggctactcgctcaaggccacaaccatgatgatccggccagcagatttctaa

SEQ ID NO: 186 - DNA, 1488 bp, Cyno
atgtggcagattgttttctttaccctgagctgtgatcttgtcttggccgcagcctataacaactttcggaagagcatggacagcataggaaagaagcagtatcaggtccagcatgggtcctgcagctacactttcctcctgccagagatggacaactgccgctcttcctccagcccctacgtgtccaatgctgtgcagagggacgcgccgctagaatatgatgactcggtgcagaggctgcaagtgctggagaacatcatggaaaacaacactcagtggctaatgaagcttgagaattatatccaggacaacatgaaaaaagaaatggtagagatacagcagaacgcagtacagaaccagacggctgtgatgatagaagtagtgacgaacctgttgaaccaaacggcggagcaaacgcggaagttaactgatgtggaagcccaagtattaaaccagaccacaagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaatcaaagaagagaaagatcagctgcaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactctaaggaccccactgttgctaaggaagaacaaatcagcttcagagactgtgctgaagtgttcaaatcaggacacaccacgaatggcgtctacacgttaacactccccaactctacagaagaggtcaaggcctactgtgacatggaagctggtggaggcgggtggacaattattcagcgacgtgaggacggcagcgttgactttcagaggacttggaaagaatataaagtgggatttggtaacccttcaggagaatattggctgggaaatgagtttgtttcacaactgactaatcagcaacgctatgtgcttaaaatacaccttaaagactgggaagggaacgaggcttactcattgtatgaacatttctatctctcaagtgaagaactcaattataggattcaccttaaaggacttacagggacagccggcaaaataagcagcatcagtcaaccaggaaatgattttagcacaaaggatgcagacaacgacaaatgtatttgcaaatgttcacaaatgctaacaggaggctggtggtttgatgcatgtggtccttccaacttgaacggaatgtactatccacaaaggcagaacacaaataagttcaacggcattaagtggtactactggaaaggctcaggctactcgctcaagggcacaaccatgatgatccgaccggcagatttctaa

SEQ ID NO: 187 - protein, 495 aa, Cyno
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEVVTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSKDPTVAKEEQISFRDCAEVFKSGHTTNGVYTLTLPNSTEEVKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDADNDKCICKCSQMLTGGWWFDACGPSNLNGMYYPQRQNTNKFNGIKWYYWKGSGYSLKGTTMMIRPADF

SEQ ID NO: 188 - protein, 496 aa, Mouse
MWQIIFLTFGWDLVLASAYSNFRKSVDSTGRRQYQVQNGPCSYTFLLPETDSCRSSSSPYMSNAVQRDAPLDYDDSVQRLQVLENILENNTQWLMKLENYIQDNMKKEMVEIQQNVVQNQTAVMIEIGTSLLNQTAAQTRKLTDVEAQVLNQTTRLELQLLQHSISTNKLEKQILDQTSEINKLQNKNSFLEQKVLDMEGKHSEQLQSMKEQKDELQVLVSKQSSVIDELEKKLVTATVNNSLLQKQQHDLMETVNSLLTMMSSPNSKSSVAIRKEEQTTFRDCAEIFKSGLTTSGIYTLTFPNSTEEIKAYCDMDVGGGGWTVIQHREDGSVDFQRTWKEYKEGFGSPLGEYWLGNEFVSQLTGQHRYVLKIQLKDWEGNEAHSLYDHFYLAGEESNYRIHLTGLTGTAGKISSISQPGSDFSTKDSDNDKCICKCSQMLSGGWWFDACGPSNLNGQYYPQKQNTNKFNGIKWYYWKGSGYSLKATTMMIRPADF

SEQ ID NO: 189 - protein, 253 aa, Mouse
METDTLLLWVLLLWVPGSTGDDYKDDDDKGILEEQTTFRDCAEIFKSGLTTSGIYTLTFPNSTEEIKAYCDMDVGGGGWTVIQHREDGSVDFQRTWKEYKEGFGSPLGEYWLGNEFVSQLTGQHRYVLKIQLKDWEGNEAHSLYDHFYLAGEESNYRIHLTGLTGTAGKISSISQPGSDFSTKDSDNDKCICKCSQMLSGGWWFDACGPSNLNGQYYPQKQNTNKFNGIKWYYWKGSGYSLKATTMMIRPADF

SEQ ID NO: 190 - protein, 253 aa, Cyno
METDTLLLWVLLLWVPGSTGDDYKDDDDKGILEEQISFRDCAEVFKSGHTTNGVYTLTLPNSTEEVKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDADNDKCICKCSQMLTGGWWFDACGPSNLNGMYYPQRQNTNKFNGIKWYYWKGSGYSLKGTTMMIRPADF

(19) United States
(12) Patent Application Publication
(10) Pub. No.: US 2020/0207845 A1
(43) Pub. Date: Jul. 2, 2020

(54) ANG2-BINDING MOLECULES

(71) Applicant: BOEHRINGER INGELHEIM INTERNATIONAL GMBH, INGELHEIM AM RHEIN (DE)

(72) Inventors: Rene Georg Ott, Perchtoldsdorf (AT); Eric Borges, Moedling (DE); Andreas Gschwind, Ingelheim am Rhein (DE); Joachim Boucneau, De Pinte (BE); Marie-Ange Buyse, Merelbeke (BE); Erik Depla, Destelbergen (BE); Pascal Merchiers, Kasterlee (BE); Frederik Stevenaert, Maarkedal (BE)

(21) Appl. No.: 16/817,700

(22) Filed: Mar. 13, 2020

Related U.S. Application Data
(63) Continuation of application No. 16/025,067, filed on Jul. 2, 2018, now abandoned.
Application No. 16/025,067 is a continuation of application No. 15/386,181, filed on Dec. 21, 2016, now abandoned.
Application No. 15/386,181 is a continuation of application No. 13/852,402, filed on Mar. 28, 2013, now abandoned.

(30) Foreign Application Priority Data
Mar. 30, 2012 (EP) 12162615.4

(51) Int. Cl. C07K 16/22; A61K 39/395; A61K 45/06; C07K 16/30

(52) U.S. Cl. CPC C07K 16/22; A61K 39/3955; A61K 45/06; C07K 16/3015; C07K 16/303; C07K 16/3038; C07K 2317/567; C07K 2317/33; C07K 2317/569; C07K 2317/92; C07K 2317/76; C07K 2317/565; C07K 16/3069

(57) ABSTRACT

Ang2-binding molecules, preferably Ang2-binding immunoglobulin single variable domains like VHHs and domain antibodies, pharmaceutical compositions containing same and their use in the treatment of diseases that are associated with Ang2-mediated effects on angiogenesis. Nucleic acids encoding Ang2-binding molecules, host cells and methods for preparing same.

### FIELD OF THE INVENTION

The invention relates to the field of human therapy, in particular cancer therapy and agents and compositions useful in such therapy.

### BACKGROUND OF THE INVENTION

As described in e.g. US 2008/0014196, WO2008101985 and US 2011/0027286, angiogenesis is the biological process whereby new blood vessels are formed and being implicated in the pathogenesis of a number of disorders, including solid tumors and metastasis as well as eye diseases. When tumors reach a critical size of approximately 1 mm³ they become dependent on angiogenesis for maintaining blood supply with oxygen and nutritients to allow for further growth. Anti-angiogenesis therapies have become an important treatment option for several types of tumors.

One of the most important pro-angiogenic factors is Angiopoietin2 (Ang2), a ligand of the Tie2 receptor (Tie2), which controls vascular remodeling by enabling the functions of other angiogenic factors, such as VEGF. Ang2 is also referred to in the art as Tie2 ligand (U.S. Pat. No. 5,643,755, Yancopoulos et al. 2000, Nature 407: 242-248).

Ang2 is primarily expressed by endothelial cells, strongly induced by hypoxia and other angiogenic factors and has been demonstrated to regulate tumor vessel plasticity, allowing vessels to respond to VEGF and FGF2 (Augustin et al., Nat Rev Mol Cell Biol. 2009 March; 10(3):165-77). Consistent with this role, the deletion or inhibition of Ang2 results in reduced angiogenesis (Falcón et al., Am J Pathol. 2009 November; 175(5):2159-70). Elevated Ang2 serum concentrations have been reported for patients with colorectal cancer, NSCLC and melanoma (Goede et al., Br J Cancer. 2010 Oct. 26; 103(9):1407-14), (Park et al., Chest. 2007 July; 132(1): 200-6), (Helfrich et al., Clin Cancer Res. 2009 Feb. 15; 15(4):1384-92). In CRC cancer Ang2 serum levels correlate with therapeutic response to anti-VEGF therapy.

The Ang-Tie system consists of 2 receptors (Tie1 and Tie2) and 3 ligands (Ang1, Ang2 and Ang4) (Augustin et al., Nat Rev Mol Cell Biol. 2009 March; 10(3):165-77). Tie2, Ang1 and Ang2 are the best studied members of this family, Tie1 is an orphan receptor and the role of Ang4 for vascular remodelling still needs to be defined. Ang2 and Ang1 mediate opposing functions upon Tie2 binding and activation. Ang2-mediated Tie2 activation results in endothelial cell activation, pericyte dissociation, vessel leakage and induction of vessel sprouting. In contrast to Ang2, Ang1 signaling maintains vessel integrity by recruitment of pericytes, thereby maintaining endothelial cell quiescence.

Angiopoietin 2 (Ang2) is a secreted, 66 kDa ligand for the Tie2 receptor tyrosine kinase (Augustin et al., Nat Rev Mol Cell Biol. 2009 March; 10(3):165-77). Ang2 consists of an N-terminal coiled-coil domain and a C-terminal fibrinogen-like domain, the latter is required for Tie2 interaction. Ang2 is primarily expressed by endothelial cells and strongly induced by hypoxia and other angiogenic factors, including VEGF. Tie2 is found on endothelial cells, haematopoietic stem cells and tumor cells. Ang2-Tie2 has been demonstrated to regulate tumor vessel plasticity, allowing vessels to respond to VEGF and FGF2.

In vitro Ang2 has been shown to act as a modest mitogen, chemoattractant and inducer of tube formation in human umbilical vein endothelial cells (HUVEC). Ang2 induces tyrosine phosphorylation of ectopically expressed Tie2 in fibroblasts and promotes downstream signaling events, such as phosphorylation of ERK-MAPK, AKT and FAK in HUVEC. An antagonistic role of Ang2 in Ang1-induced endothelial cell responses has been described.

Ang2-deficiency has been shown to result in a profound lymphatic patterning defect in mice. Although the loss of Ang2 is dispensable for embryonic vascular development, Ang2-deficient mice have persistent vascular defects in the retina and kidney. Together with the dynamic pattern of Ang2 expression at sites of angiogenesis (for example ovary), these findings indicate that Ang2 controls vascular remodeling by enabling the functions of other angiogenic factors, such as VEGF.

The Ang2-Tie2 system exerts crucial roles during the angiogenic switch and later stages of tumor angiogenesis. Ang2 expression is strongly up-regulated in the tumor-associated endothelium. Reduced growth of tumors has been observed when implanted into Ang2-deficient mice, especially during early stages of tumor growth. Therapeutic blocking of Ang2 with Ang2 mAbs has shown broad efficacy in a variety of tumor xenograft models.

As summarized in the above mentioned US 2008/0014196, angiogenesis is implicated in the pathogenesis of a number of disorders, including solid tumors and metastasis.

In the case of tumor growth, angiogenesis appears to be crucial for the transition from hyperplasia to neoplasia, and for providing nourishment for the growth and metastasis of the tumor. Folkman et al., Nature 339-58 (1989), which allows the tumor cells to acquire a growth advantage compared to the normal cells. Therefore, anti-angiogenesis therapies have become an important treatment option for several types of tumors. These therapies have focused on blocking the VEGF pathway (Ferrara et al., Nat Rev Drug Discov. 2004 May; 3(5):391-400)).

Antibodies and other peptide inhibitors that bind to Ang2 and Ang1 are mentioned in e.g. U.S. Pat. Nos. 6,166,185; 7,521,053; 7,205,275; and US Patent Application Nos.: 2006/0018909 and 2006/0246071. Furthermore, US 2011/0027286 discloses specific monoclonal Ang2 antibodies which do not antagonize the related molecule Ang1.

However, the state-of-the art monoclonal antibodies (MAbs) and fusion proteins have several shortcomings in view of their therapeutic application: To prevent their degradation, they must be stored at near freezing temperatures. Also, since they are quickly digested in the gut, they are not suited for oral administration. Another major restriction of MAbs for cancer therapy is poor transport, which results in low concentrations and a lack of targeting of all cells in a tumor.

It has been an object of the present invention to provide novel improved Ang2-binding molecules, i.e. nanobodies that block binding of Ang2 to Tie2 but not the binding of Ang1 to Tie2.

More in particular, it has been object of the invention to provide novel Ang2-binding molecules, and, specifically, Ang2-binding molecules that bind to mammalian Ang2 but not to mammalian Ang1 and, especially, to human Ang2 but not to human Ang1, wherein such molecules or polypeptides are suitable for the therapeutic and diagnostic purposes as described herein. It has been a further object of the invention to provide immunoglobulin single variable domains that specifically bind to Ang2 but not to Ang1.

Such Ang2-binding molecules, or Ang2 antagonists, are useful as pharmacologically active agents in compositions in the prevention, treatment, alleviation and/or diagnosis of diseases or conditions associated with Ang2-mediated effects on angiogenesis.

Examples for such diseases are cancer or cancerous diseases such as breast cancer, renal cell carcinoma, ovarian cancer and pancreatic cancer, eye diseases such as age-related macular degeneration and diabetic retinopathy, and/or chronic kidney diseases such as diabetic nephropathy, postrenal failure, prerenal azotemia and intrinsic renal failure.

It has been a further object of the invention to provide methods for the prevention, treatment, alleviation and/or diagnosis of such diseases, disorders or conditions, involving the use and/or administration of such Ang2-binding molecules and compositions comprising them. In particular, it is has been an object of the invention to provide such pharmacologically active Ang2-binding molecules, compositions and/or methods that provide advantages compared to the agents, compositions and/or methods currently used and/or known in the art. These advantages include improved therapeutic and/or pharmacological properties and/or other advantageous properties, e.g. for manufacturing purposes, especially as compared to conventional antibodies as those described above, or fragments thereof.

### BRIEF SUMMARY OF THE INVENTION

According to a first aspect, there is provided an Ang2-binding molecule, comprising an immunoglobulin single variable domain, wherein said immunoglobulin single variable domain comprises three complementarity determining regions CDR1, CDR2 and CDR3, wherein CDR1 has an amino acid sequence selected from amino acid sequences shown in SEQ ID NOs: 168 to 170, CDR2 has an amino acid sequence selected from amino acid sequences shown in SEQ ID Nos: 171 to 173 and CDR3 has an amino acid selected from amino acid sequences shown in SEQ ID NOs: 174 to 177.

The invention further relates to an Ang2-binding molecule consisting of said immunoglobulin single variable domain.

Furthermore, the invention relates to an Ang2-binding molecule having a sequence selected from a group consisting of SEQ ID Nos: 167, 166, 129 and 138.

According to another aspect, there is provided a nucleic acid encoding said Ang2-binding molecule as well as an expression vector comprising said nucleic acid.

The invention further relates to a host cell carrying one or more expression vectors comprising said nucleic acids.

The invention further relates to a method for producing or generating an Ang2-binding molecule according to the invention, comprising the steps of:
(a) transfecting a host cell with one or more said vectors comprising said nucleic acid molecule,
(b) culturing said host cell, and
(c) recovering and purifying said Ang2-binding molecule.

Further aspect of the invention relates to a pharmaceutical composition comprising, as the active ingredient, one or more said Ang2-binding molecules and at least a physiologically acceptable carrier

The invention further relates to applications and uses of the Ang2-binding molecules, nucleic acids, host cells, products and compositions described herein, as well as to methods for the prevention and/or treatment for diseases associated with Ang2-mediated effects on angiogenesis, preferably cancer, cancerous diseases and eye diseases.

These and other aspects, embodiments, advantages and applications of the invention will become clear from the further description hereinbelow.

### Definitions

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al., “Immunology” (2^{nd} Ed.), Gower Medical Publishing, London, N.Y. (1989), as well as to the general background art cited herein. Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The term “angiopoetin-2” or “Ang2”, unless specified as being from non-human species (e.g. “mouse Ang2”, etc) refers to human Ang2 or a biologically active fragment thereof (e.g. a fragment of the Ang2 protein which is capable of inducing angiogenesis in vitro or in vivo), i.e. to human Ang2 (variant 1) with accession no. NM 001147.2, to human Ang2 (variant 2) having accession no. NM_001118887.1 or to human Ang2 (variant 3) having accession no. NM_001118888.1, and/or their biologically active fragments thereof. The amino acid sequences of Ang2 non human species, such as mouse Ang2 and cyno Ang2 are available from protein sequence database under Accession No: NM_007426.3 (SEQ ID NO: (SEQ ID 188) and AB172643.1 (SEQ ID NO: 187), respectively.

The term “angiopoietin-1” or “Ang1”, unless specified as being from non-human species (e.g. “mouse Ang2”, etc) refers to human Ang1 or a biologically active fragment thereof (e.g. a fragment of the Ang1 protein which is capable of inducing angiogenesis in vitro or in vivo), i.e. to human Ang1 with accession no. NM_001146.3, or a biologically active fragment thereof.

Unless indicated otherwise, the terms “immunoglobulin” and “immunoglobulin sequence”-whether used herein to refer to a heavy chain antibody or to a conventional 4-chain antibody—are used as general terms to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example in terms like “immunoglobulin sequence”, “antibody sequence”, “(single) variable domain sequence”, “VHH sequence” or “protein sequence”), should generally be understood to include both the relevant amino acid sequence as well as nucleic acid sequences or nucleotide sequences encoding the same, unless the context requires a more limited interpretation.

The term “domain” (of a polypeptide or protein) as used herein refers to a folded protein structure which has the ability to retain its tertiary structure independently of the rest of the protein. Generally, domains are responsible for discrete functional properties of proteins and in many cases may be added, removed or transferred to other proteins without loss of function of the remainder of the protein and/or of the domain.

The term “immunoglobulin domain” as used herein refers to a globular region of an antibody chain (such as e.g. a chain of a conventional 4-chain antibody or of a heavy chain antibody), or to a polypeptide that essentially consists of such a globular region. Immunoglobulin domains are characterized in that they retain the immunoglobulin fold characteristic of antibody molecules, which consists of a 2-layer sandwich of about 7 antiparallel beta-strands arranged in two beta-sheets, optionally stabilized by a conserved disulphide bond.

The term “immunoglobulin variable domain” as used herein means an immunoglobulin domain essentially consisting of four “framework regions” which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) that confer specificity to an antibody for the antigen by carrying the antigen-binding site.

The term “immunoglobulin single variable domain” as used herein means an immunoglobulin variable domain which is capable of specifically binding to an epitope of the antigen without pairing with an additional variable immunoglobulin domain. One example of immunoglobulin single variable domains in the meaning of the present invention are “domain antibodies”, such as the immunoglobulin single variable domains VH and VL (VH domains and VL domains). Another example of immunoglobulin single variable domains are “VHH domains” (or simply “VHHs”) from camelids, as defined hereinafter.

In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e. by a VH-VL pair of immunoglobulin domains, which jointly bind to an epitope of the respective antigen.

“VHH domains”, also known as VHHs, V_{H}H domains, VHH antibody fragments, and VHH antibodies, have originally been described as the antigen binding immunoglobulin (variable) domain of “heavy chain antibodies” (i.e. of “antibodies devoid of light chains”; Hamers-Casterman C, Atarhouch T, Muyldermans S, Robinson G, Hamers C, Songa E B, Bendahman N, Hamers R.: “Naturally occurring antibodies devoid of light chains”; Nature 363, 446-448 (1993)). The term “VHH domain” has been chosen in order to distinguish these variable domains from the heavy chain variable domains that are present in conventional 4-chain antibodies (which are referred to herein as “V_{H} domains” or “VH domains”) and from the light chain variable domains that are present in conventional 4-chain antibodies (which are referred to herein as “V_{L} domains” or “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

In the context of the present invention, the terms VHH domain, VHH, V_{H}H domain, VHH antibody fragment, VHH antibody, as well as “Nanobody®” and “Nanobody® domain” (“Nanobody” being a trademark of the company Ablynx N.V.; Ghent; Belgium) are used interchangeably and are representatives of immunoglobulin single variable domains (having the structure FR1-CDR1-FR2-CDR2-FR3-CDR3-FR4 and specifically binding to an epitope without requiring the presence of a second immunoglobulin variable domain), and which are distinguished from VH domains by the so-called “hallmark residues”, as defined in e.g. WO2009/109635, FIG. 1.

The amino acid residues of a immunoglobulin single variable domain, e.g. a VHH, are numbered according to the general numbering for V_{H} domains given by Kabat et al. (“Sequence of proteins of immunological interest”, US Public Health Services, NIH Bethesda, Md., Publication No. 91), as applied to VHH domains from Camelids, as shown e.g. in FIG. 2 of Riechmann and Muyldermans, J. Immunol. Methods 231, 25-38 (1999). According to this numbering,
FR1 comprises the amino acid residues at positions 1-30,
CDR1 comprises the amino acid residues at positions 31-35,
FR2 comprises the amino acids at positions 36-49,
CDR2 comprises the amino acid residues at positions 50-65,
FR3 comprises the amino acid residues at positions 66-94,
CDR3 comprises the amino acid residues at positions 95-102, and
FR4 comprises the amino acid residues at positions 103-113.

However, it should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of V_{H} domains, which methods can also be applied in an analogous manner to VHH domains, are known in the art. However, in the present description, claims and figures, the numbering according to Kabat and applied to VHH domains as described above will be followed, unless indicated otherwise.

The total number of amino acid residues in a VHH domain will usually be in the range of from 110 to 120, often between 112 and 115. It should however be noted that smaller and longer sequences may also be suitable for the purposes described herein.

Immunoglobulin single variable domains, e.g. VHHs and domain antibodies, according to the preferred embodiments of the invention, have a number of unique structural characteristics and functional properties which makes them highly advantageous for use in therapy as functional antigen-binding molecules. In particular, and without being limited thereto, VHH domains (which have been “designed” by nature to functionally bind to an antigen without pairing with a light chain variable domain) can function as single, relatively small, functional antigen-binding structural units.

Due to their unique properties, immunoglobulin single variable domains, as defined herein, like VHHs or VHs (or VLs)—either alone or as part of a larger polypeptide, e.g. a biparatopic molecule—offer a number of significant advantages:
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
immunoglobulin single variable domains can be expressed from a single nucleic acid molecule and do not require any post-translational modification (like glycosylation);
immunoglobulin single variable domains can easily be engineered into multivalent and multispecific formats (as further discussed herein);
immunoglobulin single variable domains have high specificity and affinity for their target, low inherent toxicity and can be administered via alternative routes than infusion or injection;
immunoglobulin single variable domains are highly stable to heat, pH, proteases and other denaturing agents or conditions and, thus, may be prepared, stored or transported without the use of refrigeration equipments;
immunoglobulin single variable domains are easy and relatively inexpensive to prepare, both on small scale and on a manufacturing scale. For example, immunoglobulin single variable domains can be produced using microbial fermentation (e.g. as further described below) and do not require the use of mammalian expression systems, as with for example conventional antibodies;
immunoglobulin single variable domains are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) and can be administered in higher doses than such conventional 4-chain antibodies and antigen-binding fragments thereof;
VHHs have specific so-called “cavity-binding properties” (inter alia due to their extended CDR3 loop, compared to VH domains from 4-chain antibodies) and can therefore also access targets and epitopes not accessible to conventional 4-chain antibodies and antigen-binding fragments thereof;
VHHs have the particular advantage that they are highly soluble and very stable and do not have a tendency to aggregate (as with the mouse-derived antigen-binding domains described by Ward et al., Nature 341: 544-546 (1989)).

The immunoglobulin single variable domains of the invention are not limited with respect to a specific biological source from which they have been obtained or to a specific method of preparation. For example, obtaining VHHs may include the following steps:

(1) isolating the VHH domain of a naturally occurring heavy chain antibody; or screening a library comprising heavy chain antibodies or VHHs and isolating VHHs therefrom;
(2) expressing a nucleic acid molecule encoding a VHH with the naturally occurring sequence;
(3) “humanizing” (as described herein) a VHH, optionally after affinity maturation, with a naturally occurring sequence or expressing a nucleic acid encoding such humanized VHH;
(4) “camelizing” (as described below) a immunoglobulin single variable heavy domain from a naturally occurring antibody from an animal species, in particular a species of mammal, such as from a human being, or expressing a nucleic acid molecule encoding such camelized domain;
(5) “camelizing” a VH, or expressing a nucleic acid molecule encoding such a camelized VH;
(6) using techniques for preparing synthetically or semi-synthetically proteins, polypeptides or other amino acid sequences;
(7) preparing a nucleic acid molecule encoding a VHH domain using techniques for nucleic acid synthesis, followed by expression of the nucleic acid thus obtained;
(8) subjecting heavy chain antibodies or VHHs to affinity maturation, to mutagenesis (e.g. random mutagenesis or site-directed mutagenesis) and/or any other technique(s) in order to increase the affinity and/or specificity of the VHH; and/or
(9) combinations or selections of the foregoing steps.

Suitable methods and techniques for performing the above-described steps are known in the art and will be clear to the skilled person. By way of example, methods of obtaining VHH domains binding to a specific antigen or epitope have been described in WO2006/040153 and WO2006/122786.

According to specific embodiments, the immunoglobulin single variable domains of the invention or present in the polypeptides of the invention are VHH domains with an amino acid sequence that essentially corresponds to the amino acid sequence of a naturally occurring VHH domain, but that has been “humanized” or “sequence-optimized” (optionally after affinity-maturation), i.e. by replacing one or more amino acid residues in the amino acid sequence of said naturally occurring VHH sequence by one or more of the amino acid residues that occur at the corresponding position(s) in a variable heavy domain of a conventional 4-chain antibody from a human being. This can be performed using methods known in the art, which can by routinely used by the skilled person.

A humanized VHH domain may contain one or more fully human framework region sequences, and, in an even more specific embodiment, may contain human framework region sequences derived from the human germline Vh3 sequences DP-29, DP-47, DP-51, or parts thereof, or be highly homologous thereto, optionally combined with JH sequences, such as JH5. Thus, a humanization protocol may comprise the replacement of any of the VHH residues with the corresponding framework 1, 2 and 3 (FRI, FR2 and FR3) residues of germline VH genes such as DP 47, DP 29 and DP 51) either alone or in combination. Suitable framework regions (FR) of the immunoglobulin single variable domains of the invention can be selected from those as set out e.g. in WO 2006/004678 and specifically, include the so-called “KERE” and “GLEW” classes. Examples are immunoglobulin single variable domains having the amino acid sequence G-L-E-W at about positions 44 to 47, and their respective humanized counterparts. A humanized VHH domain may contain one or more fully human framework region sequences.

By way of example, a humanizing substitution for VHHs belonging to the 103 P,R,S-group and/or the GLEW-group (as defined below) is 108Q to 108L. Methods for humanizing immunoglobulin single variable domains are known in the art.

Binding immunoglobulin single variable domains with improved properties in view of therapeutic application, e.g. enhanced affinity or decreased immunogenicity, may be obtained from individual binding molecules by techniques known in the art, such as affinity maturation (for example, starting from synthetic, random or naturally occurring immunoglobulin sequences), CDR grafting, humanizing, combining fragments derived from different immunoglobulin sequences, PCR assembly using overlapping primers, and similar techniques for engineering immunoglobulin sequences well known to the skilled person; or any suitable combination of any of the foregoing, also termed “sequence optimization”, as described herein. Reference is, for example, made to standard handbooks, as well as to the further description and Examples.

If appropriate, a binding molecule with increased affinity may be obtained by affinity-maturation of another binding molecule, the latter representing, with respect to the affinity-matured molecule, the “parent” binding molecule.

Methods of obtaining VHHs that bind to a specific antigen or epitope have been described earlier, e.g. in WO2006/040153 and WO2006/122786. As also described therein in detail, VHH domains derived from camelids can be “humanized” (also termed “sequence-optimized” herein, “sequence-optimizing” may, in addition to humanization, encompass an additional modification of the sequence by one or more mutations that furnish the VHH with improved properties, such as the removal of potential post translational modification sites) by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences, and, in an even more specific embodiment, can contain human framework region sequences derived from DP-29, DP-47, DP-51, or parts thereof, optionally combined with JH sequences, such as JH5.

“Domain antibodies”, also known as “Dab”s and “dAbs” (the terms “Domain Antibodies” and “dAbs” being used as trademarks by the GlaxoSmithKline group of companies) have been described in e.g. Ward, E. S., et al.: “Binding activities of a repertoire of single immunoglobulin variable domains secreted from Escherichia coli”; Nature 341: 544-546 (1989); Holt, L. J. et al.: “Domain antibodies: proteins for therapy”; TRENDS in Biotechnology 21(11): 484-490 (2003); and WO2003/002609.

Domain antibodies essentially correspond to the VH or VL domains of antibodies from non-camelid mammals, in particular human 4-chain antibodies. In order to bind an epitope as a single antigen binding domain, i.e. without being paired with a VL or VH domain, respectively, specific selection for such antigen binding properties is required, e.g. by using libraries of human single VH or VL domain sequences.

Domain antibodies have, like VHHs, a molecular weight of approximately 13 to approximately 16 kDa and, if derived from fully human sequences, do not require humanization for e.g. therapeutical use in humans. As in the case of VHH domains, they are well expressed also in prokaryotic expression systems, providing a significant reduction in overall manufacturing cost.

Furthermore, it will also be clear to the skilled person that it is possible to “graft” one or more of the CDR's mentioned above onto other “scaffolds”, including but not limited to human scaffolds or non-immunoglobulin scaffolds. Suitable scaffolds and techniques for such CDR grafting are known in the art.

The terms “epitope” and “antigenic determinant”, which can be used interchangeably, refer to the part of a macromolecule, such as a polypeptide that is recognized by antigen-binding molecules, such as conventional antibodies or the polypeptides of the invention, and more particularly by the antigen-binding site of said molecules. Epitopes define the minimum binding site for an immunoglobulin, and thus represent the target of specificity of an immunoglobulin.

A polypeptide (such as an immunoglobulin, an antibody, an immunoglobulin single variable domain of the invention, or generally an antigen-binding molecule or a fragment thereof) that can “bind to” or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein. In this context, an Ang2-binding molecule may also be referred to as “Ang2-neutralizing”.

Generally, the term “specificity” refers to the number of different types of antigens or epitopes to which a particular antigen-binding molecule or antigen-binding protein (such as an immunoglobulin single variable domain of the invention) molecule can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule (such as an immunoglobulin, an antibody, an immunoglobulin single variable domain or a polypeptide containing it and the pertinent antigen. Avidity is related to both the affinity between an epitope and its antigen binding site on the antigen-binding molecule and the number of pertinent binding sites present on the antigen-binding molecule.

The part of an antigen-binding molecule (such as an antibody or an immunoglobulin single variable domain of the invention) that recognizes the epitope is called a “paratope”.

Unless indicated otherwise, the term “Ang2-binding molecule” includes anti-Ang2 antibodies, anti-Ang2 antibody fragments, “anti-Ang2 antibody-like molecules” and conjugates with any of these. Antibodies include, but are not limited to, monoclonal and chimerized monoclonal antibodies. The term “antibody” encompasses complete immunoglobulins, like monoclonal antibodies produced by recombinant expression in host cells, as well as Ang2-binding antibody fragments or “antibody-like molecules”, including single-chain antibodies and linear antibodies, so-called “SMIPs” (“Small Modular Immunopharmaceuticals”), as e.g described in WO02/056910. Anti-Ang2 antibody-like molecules include immunoglobulin single variable domains, as defined herein. Other examples for antibody-like molecules are immunoglobulin super family antibodies (IgSF), or CDR-grafted molecules.

“Ang2-binding molecule” refers to monovalent Ang2-binding molecules (i.e. molecules that bind to one epitope of Ang2) as well as to Ang2-binding molecules containing more than one Ang2-binding immunoglobulin single variable domain, also termed “formatted” Ang2-binding molecules. The formatted Ang-2binding molecules may, in addition to the Ang2-binding immunoglobulin single variable domains, comprise linkers and/or moieties with effector functions, e.g. half-life-extending moieties like albumin-binding immunoglobulin single variable domains, and/or a fusion partner like serum albumin and/or an attached polymer like PEG.

A formatted Ang2-binding molecule may, albeit less preferred, also comprise two identical Ang2-binding immunoglobulin single variable domains or two different immunoglobulin single variable domains that recognize the same or overlapping epitopes. In this case, the two immunoglobulin single variable domains may bind to the same or an overlapping epitope in each of the two monomers that form the Ang2 dimer. Experimental data including competitive ELISA assay discloses a significant improvement in the potency of the formatted Ang2 dimers when compared to the individual building blocks of mono Ang2-binding molecules (data not shown).

Typically, the Ang2-binding molecules of the invention will bind with a dissociation constant (K_{D}) of 10E-5 to 10E-14 moles/liter (M) or less, and preferably 10E-7 to 10E-14 moles/liter (M) or less, more preferably 10E-8 to 10E-14 moles/liter, and even more preferably 10E-11 to 10E-13 (as measured in a Biacore or in a KinExA assay), and/or with an association constant (K_{A}) of at least 10E7 ME-1, preferably at least 10E8 ME-1, more preferably at least 10E9 ME-1, such as at least 10E11 ME-1. Any K_{D} value greater than 10E-4 M is generally considered to indicate non-specific binding. Preferably, a polypeptide of the invention will bind to the desired antigen, i.e. Ang2, with a K_{D} less than 500 nM, preferably less than 200 nM, more preferably less than 10 nM, such as less than 500 pM. Specific binding of an antigen-binding protein to an antigen or epitope can be determined in any suitable manner known per se, including, for example, the assays described herein, Scatchard analysis and/or competitive binding assays, such as radioimmunoassays (RIA), enzyme immunoassays (EIA) and sandwich competition assays, and the different variants thereof known per se in the art.

Amino acid residues will be indicated according to the standard three-letter or one-letter amino acid code, as generally known and agreed upon in the art. When comparing two amino acid sequences, the term “amino acid difference” refers to insertions, deletions or substitutions of the indicated number of amino acid residues at a position of the reference sequence, compared to a second sequence. In case of substitution(s), such substitution(s) will preferably be conservative amino acid substitution(s), which means that an amino acid residue is replaced with another amino acid residue of similar chemical structure and which has little or essentially no influence on the function, activity or other biological properties of the polypeptide. Such conservative amino acid substitutions are well known in the art, for example from WO98/49185, wherein conservative amino acid substitutions preferably are substitutions in which one amino acid within the following groups (i)-(v) is substituted by another amino acid residue within the same group: (i) small aliphatic, nonpolar or slightly polar residues: Ala, Ser, Thr, Pro and Gly; (ii) polar, negatively charged residues and their (uncharged) amides: Asp, Asn, Glu and Gin; (iii) polar, positively charged residues: His, Arg and Lys; (iv) large aliphatic, nonpolar residues: Met, Leu, lie, Val and Cys; and (v) aromatic residues: Phe, Tyr and Trp. Particularly preferred conservative amino acid substitutions are as follows:

Ala into Gly or into Ser; Arg into Lys; Asn into Gin or into His; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp or into Phe; Val into lie or into Leu.

A polypeptide or nucleic acid molecule is considered to be “(in) essentially isolated (form)”—for example, when compared to its native biological source and/or the reaction medium or cultivation medium from which it has been obtained—when it has been separated from at least one other component with which it is usually associated in said source or medium, such as another protein/polypeptide, another nucleic acid, another biological component or macromolecule or at least one contaminant, impurity or minor component. In particular, a polypeptide or nucleic acid molecule is considered “essentially isolated” when it has been purified at least 2-fold, in particular at least 10-fold, more in particular at least 100-fold, and up to 1000-fold or more. A polypeptide or nucleic acid molecule that is “in essentially isolated form” is preferably essentially homogeneous, as determined using a suitable technique, such as a suitable chromatographical technique, such as polyacrylamide gel electrophoresis.

The term “N-terminus” (also known as the amino-terminus, NH₂-terminus, N-terminal end or amine-terminus) refers to the start of a protein/polypeptide (i.e. Ang2-binding molecule) terminated by an amino acid with a free amine group (—NH₂). The convention for writing peptide sequences is to put the N-terminus on the left and write the sequence from N- to C-terminus. When the protein is translated from messenger RNA, it is created from N-terminus to C-terminus.

“Sequence identity” between two Ang2-binding molecule sequences indicates the percentage of amino acids that are identical between the sequences. It may be calculated or determined as described in paragraph f) on pages 49 and 50 of WO08/020079. “Sequence similarity” indicates the percentage of amino acids that either is identical or that represent conservative amino acid substitutions.

Alternative methods for numbering the amino acid residues of V_{H} domains, which methods can also be applied in an analogous manner to VHH domains, are known in the art. However, in the present description, claims and figures, the numbering according to Kabat and applied to VHH domains as described above will be followed, unless indicated otherwise.

An “affinity-matured” Ang2-binding molecule, in particular a VHH or a domain antibody, has one or more alterations in one or more CDRs which result in an improved affinity for Ang2, as compared to the respective parent Ang2-binding molecule. Affinity-matured Ang2-binding molecules of the invention may be prepared by methods known in the art, for example, as described by Marks et al., 1992, Biotechnology 10:779-783, or Barbas, et al., 1994, Proc. Nat. Acad. Sci, USA 91: 3809-3813.; Shier et al., 1995, Gene 169:147-155; Yelton et al., 1995, Immunol. 155: 1994-2004; Jackson et al., 1995, J. Immunol. 154(7):3310-9; and Hawkins et al., 1992, J. Mol. Biol. 226(3): 889 896; KS Johnson and RE Hawkins, “Affinity maturation of antibodies using phage display”, Oxford University Press 1996.

For the present invention, an “amino acid sequences of SEQ ID NO: x”: includes, if not otherwise stated, an amino acid sequence that is 100% identical with the sequence shown in the respective SEQ ID NO: x;
a) amino acid sequences that have at least 80% amino acid identity with the sequence shown in the respective SEQ ID NO: x;
b) amino acid sequences that have 3, 2, or 1 amino acid differences with the sequence shown in the respective SEQ ID NO: x.

The terms “cancer” and “cancerous” refer to or describe the physiological condition in mammals that is typically characterized by unregulated cell growth/proliferation. Examples of cancer to be treated with an Ang2-binding molecule of the invention include but are not limited to carcinoma, lymphoma, blastoma, sarcoma, and leukemia. More particular examples of such cancers, include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, renal cell carcinoma, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplasties include but are not limited those described above.

Non-neoplastic disorders include, but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3^{rd} spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osier-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

The term “eye diseases” refers to proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization.

The term “chronic kidney diseases” refers to diabetic nephropathy, postrenal failure, prerenal azotemia and intrinsic renal failure.

### DETAILED DESCRIPTION OF THE INVENTION

In a first aspect, the present invention relates to an Ang2-binding molecule comprising an immunoglobulin single variable domain, wherein said immunoglobulin single variable domain comprises three complementarity determining regions CDR1, CDR2 and CDR3, wherein CDR1 has an amino acid sequence selected from amino acid sequences shown in SEQ ID NOs: 168 to 170, CDR2 has an amino acid sequence selected from amino acid sequences shown in SEQ ID NOs: 171 to 173 and CDR3 has an amino acid selected from amino acid sequences SEQ ID NOs: 174 to 177.

CDR1 has a sequence selected from

SEQ ID NO: 168
DYAIG
SEQ ID NO: 169
DYALG
SEQ ID NO: 170
YYAIG

CDR2 has a sequence selected from

SEQ ID NO: 171
AIRSSGGSTYYADSVKG
SEQ ID NO: 172
CIRCSGGSTYYADSVKG
SEQ ID NO: 173
CISSSGGITYYADSVKG

CDR3 has a sequence selected from

SEQ ID NO: 174
VPAGRLRFGEQWYPLYEYDA
SEQ ID NO: 175
VPAGRLRYGEQWYPIYEYDA
SEQ ID NO: 176
SIVPRSKLEPYEYDA
SEQ ID NO: 177
DSGGYIDYDCSGLGYDY

According to preferred embodiments, the Ang2-binding molecule comprises an immunoglobulin single variable domain, wherein
(a) CDR1 has an amino acid sequence shown in SEQ ID NO: 168, CDR2 has an amino acid sequence shown in SEQ ID NO: 171 and CDR3 has an amino acid sequence shown in SEQ ID NO: 174, or wherein
(b) CDR1 has an amino acid sequence shown in SEQ ID NO: 168, CDR2 has an amino acid sequence shown in SEQ ID NO: 171 and CDR3 has an amino acid sequence shown in SEQ ID NO: 175, or wherein
(c) CDR1 has an amino acid sequence shown in SEQ ID NO: 169, CDR2 has an amino acid sequence shown in SEQ ID NO: 172 and CDR3 has an amino acid sequence shown in SEQ ID NO: 176, or wherein
(d) CDR1 has an amino acid sequence shown in SEQ ID NO: 170, CDR2 has an amino acid sequence shown in SEQ ID NO: 173 and CDR3 has an amino acid sequence shown in SEQ ID NO: 177.

According to another preferred embodiments, the Ang2-binding molecule comprises an immunoglobulin single variable domain, wherein said immunoglobulin single variable domain is a VHH or a domain antibody.

According to still more preferred embodiments, the Ang2-binding molecule comprises an immunoglobulin single variable domain, wherein said immunoglobulin single variable domain is a VHH.

According to specific embodiments, said VHH consists of an immunoglobulin single variable domain having a sequence selected from a group consisting of SEQ ID NOs: 167, 166, 129 and 138.

In another aspect, the present invention relates to an Ang2-binding molecule consisting of said immunoglobulin single variable domain.

The sequence of the Ang2 binders according to the invention can be modified at their N-terminus (i.e. deletion or exchange of the first amino acid) without significant reduction of their binding activity. This modification enhances the co-/post-translational cleavage of N-terminal methionine during intracellular/cytoplasmic expression in bacterial hosts (e.g. but not limited to Escherichia coli).

In one aspect said VHH consisting of an immunoglobulin single variable domain has a modification or exchange on N terminus, wherein said modification is a deletion of a first amino acid and said exchange is a replacement of the first amino acid by another amino acid.

In one of preferred embodiments the first amino acid on N terminus is Valine (V) or Aspartic acid (D) replaced by e.g. by Alanine (A).

Ang2-binding components with improved properties in view of therapeutic application, e.g. enhanced affinity or decreased immunogenicity, may be obtained from individual Ang2-binding components of the invention by techniques such as affinity maturation (for example, starting from synthetic, random or naturally occurring immunoglobulin sequences), CDR grafting, humanizing, combining fragments derived from different immunoglobulin sequences, PCR assembly using overlapping primers, and similar techniques for engineering immunoglobulin sequences well known to the skilled person; or any suitable combination of any of the foregoing. Reference is, for example, made to standard handbooks, as well as to the further description and Examples.

Preferably, an Ang2-binding component of the invention with increased affinity is obtained by affinity-maturation of another Ang2-binding component, the latter representing, with respect to the affinity-matured molecule, the “parent” Ang2-binding component.

Thus, in yet another preferred embodiment, an Ang2-binding molecule of the invention is an immunoglobulin single variable domain that has been obtained by affinity maturation of a parent immunoglobulin single variable domain defined above.

In yet another preferred embodiment, the invention relates to an immunoglobulin single variable domain obtained by affinity-maturation of a VHH.

In a preferred embodiment, the invention relates to an immunoglobulin single variable domain that has been used for humanization of a VHH with an amino acid sequence shown in SEQ ID NOs: 1, 17 and 80.

In another preferred embodiment, the invention relates to an immunoglobulin single variable domain that has been obtained by humanization of a VHH with an amino acid sequence shown in SEQ ID NOs: 127, 132 and 146.

The invention also relates to Ang2-binding molecules that have been obtained by affinity maturation and/or sequence optimization of an above-defined VHH, e.g. to a VHH that has been obtained by sequence optimization of a VHH having an amino acid sequence shown as SEQ ID NOs: 167, 166, 129 and 138.

In another more preferred embodiment, the invention relates to an immunoglobulin single variable domain that has been obtained by affinity maturation of a VHH with an amino acid sequence shown in SEQ ID NO: 167.

In even more preferred embodiment, the invention relates to an immunoglobulin single variable domain that has been obtained by affinity maturation of a VHH with an amino acid sequence shown in SEQ ID NO: 166.

In yet another preferred embodiment, the invention relates to an immunoglobulin single variable domain that has been obtained by affinity maturation of a VHH with an amino acid sequence shown in SEQ ID NO: 129.

In still further preferred embodiment, the invention relates to an immunoglobulin single variable domain that has been obtained by affinity maturation of a VHH with an amino acid sequence shown in SEQ ID NO: 138.

In yet another embodiment, the representatives of the class of Ang2-binding immunoglobulin single variable domains of the invention or present in the polypeptides of the invention have amino acid sequences that correspond to the amino acid sequence of a naturally occurring VH domain that has been “camelized”, i.e. by replacing one or more amino acid residues in the amino acid sequence of a naturally occurring variable heavy chain from a conventional 4-chain antibody by one or more amino acid residues that occur at the corresponding position(s) in a VHH domain of a heavy chain antibody. This can be performed in a manner known per se, which will be clear to the skilled person, and reference is additionally be made to WO 1994/04678.

Such camelization may preferentially occur at amino acid positions which are present at the VH-VL interface and at the so-called Camelidae Hallmark residues (see for example also WO 1994/04678). A detailed description of such “humanization” and “camelization” techniques and preferred framework region sequences consistent therewith can additionally be taken from e.g. pp. 46 and pp. 98 of WO 2006/040153 and pp. 107 of WO 2006/122786.

The Ang2-binding components of the invention, e.g. immunoglobulin single variable domains and or polypeptides containing them, have specificity for Ang2 in that they comprise one or more immunoglobulin single variable domains specifically binding to one or more epitopes within the Ang2 molecule.

Specific binding of an Ang2-binding component to its antigen Ang2 can be determined in any suitable manner known per se, including, for example, the assays described herein, Scatchard analysis and/or competitive binding assays, such as radioimmunoassays (RIA), enzyme immunoassays (EIA and ELISA) and sandwich competition assays, and the different variants thereof known per se in the art.

With regard to the antigen Ang2, an Ang2-binding component of the invention, e.g. an immunoglobulin single variable domain, is not limited with regard to the species. Thus, the immunoglobulin single variable domains of the invention or polypeptides containing them preferably bind to human Ang2, if intended for therapeutic purposes in humans. However, immunoglobulin single variable domains that bind to Ang2 from another mammalian species, or polypeptides containing them, are also within the scope of the invention. An immunoglobulin single variable domain of the invention binding to one species form of Ang2 may cross-react with Ang2 from one or more other species. For example, immunoglobulin single variable domains of the invention binding to human Ang2 may exhibit cross reactivity with Ang2 from one or more other species of primates and/or with Ang2 from one or more species of animals that are used in animal models for diseases, for example monkey (in particular Cynomolgus or Rhesus), mouse, rat, rabbit, pig, dog or) and in particular in animal models for diseases and disorders associated with Ang2-mediated effects on angiogenesis (such as the species and animal models mentioned herein). Immunoglobulin single variable domains of the invention that show such cross-reactivity are advantageous in a research and/or drug development, since it allows the immunoglobulin single variable domains of the invention to be tested in acknowledged disease models such as monkeys, in particular Cynomolgus or Rhesus, or mice and rats.

Also, the Ang2-binding components of the invention are not limited to or defined by a specific domain or an antigenic determinant of Ang2 against which they are directed. Preferably, in view of cross-reactivity with one or more Ang2 molecules from species other than human that is/are intended for use as an animal model during development of a therapeutic Ang2 antagonist, a Ang2-binding component recognizes an epitope in a region of the Ang2 of interest that has a high degree of identity with human Ang2. By way of example, in view of using a mouse model, an immunoglobulin single variable domain of the invention recognizes an epitope which is, totally or in part, located within the FLD-domain published in Kim H-Z, Jung K, Kim H M, Cheng Y, and Koh G Y (2009). A designed angiopoietin-2 variant, pentameric COMP-Ang2, strongly activates Tie2 receptor and stimulated angiogensis. Biochim Biophys Acta 1793, 772-780.

Therefore, according to a preferred embodiment, the invention relates to a Ang2-binding component, in particular an immunoglobulin single variable domain or a polypeptide containing same, wherein said immunoglobulin single variable domain is selected from the group that binds to an epitope that is totally or partially contained within the FLD domain (SEQ ID NOs: 188 to 190).

Preferably, an immunoglobulin single variable domain of the invention binds to Ang2 with an affinity less than 500 nM, preferably less than 200 nM, more preferably less than 10 nM, such as less than 500 pM (as determined by Surface Plasmon Resonance analysis).

Preferably, the immunoglobulin single variable domains of the invention have IC₅₀ values, as measured in a competition ELISA assay in the range of 10⁻⁶ to 10⁻¹⁰ moles/litre or less, more preferably in the range of 10⁻⁸ to 10⁻¹⁰ moles/litre or less and even more preferably in the range of 10⁻⁹ to 10⁻¹⁰ moles/litre or less.

According to a non-limiting but preferred embodiment of the invention, Ang2-binding immunoglobulin single variable domains of the invention or polypeptides containing them bind to Ang2 with an dissociation constant (K_{D}) of 10⁻⁵ to 10⁻¹² moles/liter (M) or less, and preferably 10^{0.7} to 10⁻¹² moles/liter (M) or less and more preferably 10⁻⁸ to 10⁻¹² moles/liter (M), and/or with an association constant (K_{A}) of at least 10⁷ M⁻¹, preferably at least 10⁸ M⁻¹, more preferably at least 10⁹ M⁻¹, such as at least 10¹² M⁻¹; and in particular with a K_{D} less than 500 nM, preferably less than 200 nM, more preferably less than 10 nM, such as less than 500 pM. The K_{D} and K_{A} values of the immunoglobulin single variable domain of the invention against Ang2 can be determined.

According to another embodiment, the immunoglobulin single variable domains are domain antibodies, as defined herein.

Immunoglobulin single variable domains present in the monospecific binding molecules of the invention have sequences that correspond to the amino acid sequence of a naturally occurring VH domain that has been “camelized”, i.e. by replacing one or more amino acid residues in the amino acid sequence of a naturally occurring variable heavy chain from a conventional 4-chain antibody by one or more amino acid residues that occur at the corresponding position(s) in a VHH domain of a heavy chain antibody. This can be performed in a manner known per se, which will be clear to the skilled person, and reference is additionally be made to WO 94/04678. Such camelization may preferentially occur at amino acid positions which are present at the VH-VL interface and at the so-called Camelidae Hallmark residues (see for example also WO 94/04678). A detailed description of such “humanization” and “camelization” techniques and preferred framework region sequences consistent therewith can additionally be taken from e.g. pp. 46 and pp. 98 of WO 2006/040153 and pp. 107 of WO 2006/122786.

The binding components have specificity for Ang2, in that they comprise in a preferred embodiment one immunoglobulin single variable domains specifically binding to one or more epitopes within the Ang2 molecule.

Specific binding of a binding component to its antigen Ang2 can be determined in any suitable manner known per se, including, for example, the assays described herein, Scatchard analysis and/or competitive binding assays, such as radioimmunoassays (RIA), enzyme immunoassays (EIA and ELISA) and sandwich competition assays, and the different variants thereof known per se in the art.

With regard to the antigen Ang2, an immunoglobulin single variable domain is not limited with regard to the species. Thus, the immunoglobulin single variable domains preferably bind to human Ang2, if intended for therapeutic purposes in humans. However, immunoglobulin single variable domains that bind to Ang2, from another mammalian species, or polypeptides containing them, are also within the scope of the invention. An immunoglobulin single variable domain binding to one species form of Ang2 may cross-react with the respective antigen from one or more other species. For example, immunoglobulin single variable domains binding to the human antigen may exhibit cross reactivity with the respective antigen from one or more other species of primates and/or with the antigen from one or more species of animals that are used in animal models for diseases, for example monkey (in particular Cynomolgus or Rhesus), mouse, rat, rabbit, pig, dog or) and in particular in animal models for diseases and disorders that can be modulated by inhibition of Ang2 (such as the species and animal models mentioned herein). Immunoglobulin single variable domains of the invention that show such cross-reactivity are advantageous in a research and/or drug development, since it allows the immunoglobulin single variable domains of the invention to be tested in acknowledged disease models such as monkeys, in particular Cynomolgus or Rhesus, or mice and rats.

Also, the binding components are not limited to or defined by a specific domain or an antigenic determinant of the antigen against which they are directed. Preferably, in view of cross-reactivity with one or more antigen molecules from species other than human that is/are intended for use as an animal model during development of a therapeutic Ang2 antagonist, a binding component recognizes an epitope in a region of the respective antigen that has a high degree of identity with the human antigen. By way of example, in view of using a mouse model, an anti-Ang2 immunoglobulin single variable domain contained in the monospecific binding molecules of the invention recognizes an epitope which is, totally or in part, located within the EGF-2 domain of Ang2, which shows a high identity between human and mouse.

Therefore, according to a preferred embodiment, the monospecific binding molecule of the invention comprises an Ang2-binding molecule which is an immunoglobulin single variable domain that is selected from the group consisting of SEQ ID NOs: 167, 166, 129 and 138 that binds to an epitope that is totally or partially contained within the FLD domain.

The present invention also relates to a nucleic acid encoding the Ang2-binding molecule according to the invention.

In a preferred embodiment an Ang2-binding molecule according to the invention when expressed in Escherichia coli is encoded by a nucelotide seuquence selected from:

SEQ ID NO: 178
GAGGTACAGCTGGTCGAGTCAGGTGGCGGATTAGTGCAGCCTGGGGGTTC
TCTGCGCTTATCTTGTGCCGCATCAGGCTTCACACTGGATGACTACGCCA
TCGGCTGGTTCCGGCAAGCGCCTGGAAAAGAACGCGAAGGTGTTTCAGCA
ATCCGTTCAAGCGGTGGTTCAACATATTACGCCGACTCTGTTAAAGGACG
CTTCACCATTAGCTCCGACAATAGTAAAAATACAGTCTACTTACAAATGA
ACAGTTTACGCCCAGAAGATACTGCGGTATACTATTGCGCTGCCGTGCCT
GCTGGTCGCTTACGCTTTGGCGAGCAATGGTATCCTCTGTACGAGTACGA
CGCCTGGGGACAGGGTACGCTGGTAACGGTTTCAAGC
SEQ ID NO: 179
GAAGTGCAACTGGTTGAGTCAGGTGGCGGACTGGTGCAACCGGGTGGTTC
ACTGCGCCTGAGTTGCGCAGTTAGCGGTATTACCCTGGATGATTATGCAA
TTGGTTGGTTTCGCCAAGCCCCAGGCAAAGAGCGTGAAGGCGTTAGCGCA
ATTCGTAGCAGCGGTGGTAGCACCTATTATGCCGATTCAGTTAAAGGCCG
TTTTACGATCAGCAGCGATAACAGTAAAAACACGGTTTATCTGCAAATGA
ACTCATTACGTCCAGAGGACACTGCAGTTTACTATTGCGCAGCAGTTCCG
GCAGGTCGTCTGCGTTATGGTGAACAGTGGTATCCGATTTATGAATATGA
TGCATGGGGTCAAGGTACACTGGTTACAGTGAGTAGC
SEQ ID NO: 180
GAAGTGCAGTTAGTCGAAAGTGGCGGAGGCCTGGTACAACCTGGTGGCAG
TCTGCGCTTATCTTGTGCCGCTTCAGGTTTTACATTCGACGACTACGCCC
TGGGGTGGTTCCGGCAAGCGCCTGGAAAAGAACGTGAGGGCGTTTCATGC
ATTCGTTGTTCAGGTGGTTCAACCTATTATGCCGATAGTGTAAAAGGTCG
GTTCACCATTAGTAGCGACAATAGCAAGAATACAGTCTATCTGCAAATGA
ACTCTTTACGTCCTGAAGATACTGCGGTGTACTACTGCGCTGCATCAATC
GTTCCTCGTTCAAAACTTGAACCTTACGAGTACGACGCCTGGGGTCAGGG
TACGTTAGTAACGGTGTCAAGC
And
SEQ ID NO: 181
GAAGTGCAACTGGTTGAGTCAGGTGGCGGTTTAGTGCAACCGGGTGGTTC
ACTGCGCCTGAGTTGCGCAGCCAGCGGTTTTGCACTGGATTATTATGCAA
TTGGTTGGTTTCGCCAAGCCCCAGGCAAAGAGCGTGAAGGCGTTAGCTGT
ATTAGCAGCAGCGGTGGTATTACCTATTATGCCGATTCAGTTAAAGGCCG
TTTTACGATCTCTCGTGATAATAGTAAAAACACGGTTTACCTGCAGATGA
ACTCATTAAGACCAGAGGACACTGCAGTTTACTATTGTGCAACCGATAGC
GGTGGCTATATTGATTATGATTGTAGCGGTCTGGGCTACGATTATTGGGG
ACAAGGTACGCTGGTGACAGTTAGCAGC

The invention relates to nucleic acid molecules that encode monospecific binding molecules of the invention. Such nucleic acid molecules will also be referred to herein as “nucleic acids of the invention” and may also be in the form of a genetic construct, as defined herein. A nucleic acid of the invention may be genomic DNA, cDNA or synthetic DNA (such as DNA with a codon usage that has been specifically adapted for expression in the intended host cell or host organism). According to one embodiment of the invention, the nucleic acid of the invention is in essentially isolated form, as defined hereabove.

Further aspect of the invention relates to an expression vector comprising the nucleic acid molecule encoding said Ang2-binding molecule according to invention.

In preferred embodiments, the nucleic acid of the invention may also be in the form of, may be present in and/or may be part of a vector, such as for example a plasmid, cosmid or YAC

The vector may especially be an expression vector, i.e. a vector that can provide for expression of the monospecific binding molecule in vitro and/or in vivo (i.e. in a suitable host cell, host organism and/or expression system). Such expression vector generally comprises at least one nucleic acid of the invention that is operably linked to one or more suitable regulatory elements, such as promoter(s), enhancer(s), terminator(s), and the like. Such elements and their selection in view of expression of a specific sequence in a specific host are common knowledge of the skilled person. Specific examples of regulatory elements and other elements useful or necessary for expressing bispecific binding molecules of the invention, such as promoters, enhancers, terminators, integration factors, selection markers, leader sequences, reporter genes, and the like, are disclosed e.g. on pp. 131 to 133 of WO 2006/040153.

Such vectors express or are capable of expressing one or more monospecific binding molecules of the invention; and/or contain a nucleic acid of the invention.

The nucleic acids of the invention may be prepared or obtained in a manner known per se (e.g. by automated DNA synthesis and/or recombinant DNA technology), based on the information on the amino acid sequences for the polypeptides of the invention given herein, and/or can be isolated from a suitable natural source.

In another aspect, the invention relates to a host cell carrying one or more expression vectors comprising a nucleic acid molecule encoding the Ang2-binding molecule according to the invention.

According to a particularly preferred embodiment, said host cells are bacterial cells; other useful cells are yeast cells, fungal cells or mammalian cells.

Suitable bacterial cells include cells from gram-negative bacterial strains such as strains of Escherichia coli, Proteus, and Pseudomonas, and gram-positive bacterial strains such as strains of Bacillus, Streptomyces, Staphylococcus, and Lactococcus. Suitable fungal cell include cells from species of Trichoderma, Neurospora, and Aspergillus. Suitable yeast cells include cells from species of Saccharomyces (for example Saccharomyces cerevisiae), Schizosaccharomyces (for example Schizosaccharomyces pombe), Pichia (for example Pichia pastoris and Pichia methanolica), and Hansenula.

Suitable mammalian cells include for example CHO cells, BHK cells, HeLa cells, COS cells, and the like. However, amphibian cells, insect cells, plant cells, and any other cells used in the art for the expression of heterologous proteins can be used as well.

The invention further provides methods of manufacturing a monospecific binding molecule of the invention, such methods generally comprising the steps of:
culturing host cells comprising a nucleic acid capable of encoding a monospecific binding molecule under conditions that allow expression of the monospecific binding molecule of the invention; and
recovering or isolating the polypeptide expressed by the host cells from the culture; and
optionally further purifying and/or modifying and/or formulating the monospecific binding molecule of the invention.

The preferred embodiment represents a method for producing an Ang2 binding molecule having sequence SEQ ID NOs: 167, 166, 129 and 138 comprising the steps of:
(a) transfecting a host cell with one or more said vectors
(b) culturing said host cell, and
(c) recovering and purifying said Ang2 binding molecule.

For production on an industrial scale, preferred host organisms include strains of E. coli, Pichia pastoris, and S. cerevisiae that are suitable for large scale expression, production and fermentation, and in particular for large scale pharmaceutical expression, production and fermentation.

The choice of the specific expression system depends in part on the requirement for certain post-translational modifications, more specifically glycosylation. The production of a monospecific binding molecule of the invention for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression.

Monospecific binding molecules of the invention may be produced either in a cell as set out above intracellullarly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

Methods and reagents used for the recombinant production of polypeptides, such as specific suitable expression vectors, transformation or transfection methods, selection markers, methods of induction of protein expression, culture conditions, and the like, are known in the art. Similarly, protein isolation and purification techniques useful in a method of manufacture of a polypeptide of the invention are well known to the skilled person.

These peptides correspond to CDR3s derived from the VHHs of the invention. They, in particular the nucleic acid molecules encoding them, are useful for CDR grafting in order to replace a CDR3 in an immunoglobulin chain, or for insertion into a non-immunoglobulin scaffold, e.g. a protease inhibitor, DNA-binding protein, cytochrome b562, a helix-bundle protein, a disulfide-bridged peptide, a lipocalin or an anticalin, thus conferring target-binding properties to such scaffold. The method of CDR-grafting is well known in the art and has been widely used, e.g. for humanizing antibodies (which usually comprises grafting the CDRs from a rodent antibody onto the Fv frameworks of a human antibody).

In order to obtain an immunoglobulin or a non-immunoglobulin scaffold containing a CDR3 of the invention, the DNA encoding such molecule may be obtained according to standard methods of molecular biology, e.g. by gene synthesis, by oligonucleotide annealing or by means of overlapping PCR fragments, as e.g. described by Daugherty et al., 1991, Nucleic Acids Research, Vol. 19, 9, 2471-2476. A method for inserting a VHH CDR3 into a non-immunoglobulin scaffold has been described by Nicaise et al., 2004, Protein Science, 13, 1882-1891.

The invention further relates to a product or composition containing or comprising at least one monospecific binding molecule of the invention and optionally one or more further components of such compositions known per se, i.e. depending on the intended use of the composition.

In further aspect, the invention relates to use of a monospecific binding molecule of the invention as a medicament.

In still another aspect, the invention relates to use of a monospecific binding molecule of the invention for method of treating of cancer, cancerous or eye diseases.

For pharmaceutical use, a monospecific binding molecule of the invention or a polypeptide containing same may be formulated as a pharmaceutical preparation or composition comprising at least one monospecific binding molecule of the invention and at least one physiologically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one monospecific binding molecule, in particular one immunoglobulin single variable domain of the invention or a polypeptide containing same and at least one suitable carrier, diluent or excipient (i.e. suitable for pharmaceutical use), and optionally one or more further active substances.

The monospecific binding molecules of the invention may be formulated and administered in any suitable manner known per se: Reference, in particular for the immunoglobulin single variable domains, is for example made to WO 2004/041862, WO 2004/041863, WO 2004/041865, WO 2004/041867 and WO 2008/020079, as well as to the standard handbooks, such as Remington's Pharmaceutical Sciences, 18^{th} Ed., Mack Publishing Company, USA (1990), Remington, the Science and Practice of Pharmacy, 21^{th} Edition, Lippincott Williams and Wilkins (2005); or the Handbook of Therapeutic Antibodies (S. Dubel, Ed.), Wiley, Weinheim, 2007 (see for example pages 252-255).

For example, an immunoglobulin single variable domain of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

Thus, the monospecific binding molecule of the invention may be systemically administered, e.g., orally, in combination with a pharmaceutically acceptable vehicle such as an inert diluent or an assimilable edible carrier. For oral therapeutic administration, the bispecific binding molecule of the invention may be combined with one or more excipients and used in the form of ingestible tablets, buccal tablets, troches, capsules, elixirs, suspensions, syrups, wafers, and the like. Such compositions and preparations should contain at least 0.1% of the Ang2-binding molecule of the invention. Their percentage in the compositions and preparations may, of course, be varied and may conveniently be between about 2 to about 60% of the weight of a given unit dosage form. The amount of the bispecific binding molecule of the invention in such therapeutically useful compositions is such that an effective dosage level will be obtained.

The tablets, pills, capsules, and the like may also contain binders, excipients, disintegrating agents, lubricants and sweetening or flavouring agents, for example those mentioned on pages 143-144 of WO 08/020079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the bispecific binding molecules of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the bispecific binding molecules of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The monospecific binding molecules of the invention may also be administered intravenously or intraperitoneally by infusion or injection, as further described on pages 144 and 145 of WO 2008/020079.

For topical administration of the monospecific binding molecules of the invention, it will generally be desirable to administer them to the skin as compositions or formulations, in combination with a dermatologically acceptable carrier, which may be a solid or a liquid, as further described on page 145 of WO 2008/020079.

Generally, the concentration of the monospecific binding molecules of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the monospecific binding molecules of the invention required for use in treatment will vary not only with the particular monospecific binding molecule selected, but also with the route of administration, the nature of the condition being treated and the age and condition of the patient and will be ultimately at the discretion of the attendant physician or clinician. Also, the dosage of the monospecific binding molecules of the invention varies depending on the target cell, tumor, tissue, graft, or organ. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen may include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

According to a further embodiment, the invention relates to the use of monospecific binding molecules of the invention, e.g. immunoglobulin single variable domains or polypeptides containing them, for therapeutic purposes, such as
for the prevention, treatment and/or alleviation of a disorder, disease or condition, especially in a human being, that is associated with Ang2-mediated and/or Ang2-related effects on angiogenesis or that can be prevented, treated or alleviated by modulating the Notch signaling pathway and/or the Tie2 signalling pathway with a monospecific binding molecule according to the invention,
in a method of treatment of a patient in need of such therapy, such method comprising administering, to a subject in need thereof, a pharmaceutically active amount of at least one monospecific binding molecule of the invention, e.g. an immunoglobulin single variable domain, or a pharmaceutical composition containing same;
for the preparation of a medicament for the prevention, treatment or alleviation of disorders, diseases or conditions associated with Ang2-mediated and/or Ang2-mediated effects on angiogenesis;
as an active ingredient in a pharmaceutical composition or medicament used for the above purposes.

According to a specific aspect, said disorder, disease or condition is a cancer or cancerous disease, as defined herein.

In preferred embodiments, the invention relates to said pharmaceutical composition for treatment of cancer and cancerous diseases, such as breast, renal cell carcinoma, ovarian cancer and pancreatic cancer.

According to another aspect, the disease is an eye disease associated with Ang2-mediated and/or Ang2-mediated effects on angiogenesis or which can be treated or alleviated by modulating the Notch signaling pathway and/or the Tie2 signalling pathway with a monospecific binding molecule.

In another preferred embodiments, the invention relates to said pharmaceutical composition for treatment of eye diseases, such as age-related macular degeneration and diabetic retinopathy.

In still another preferred embodiments, the invention relates to said pharmaceutical composition for treatment of chronic kidney diseases.

Depending on cancer/cancerous diseases, eye diseases and/or chronic kidney diseases to be treated, a monospecific binding molecule of the invention may be used on its own or in combination with one or more additional therapeutic agents.

In preferred embodiments, the invention relates to the pharmaceutical composition comprising, as the active ingredient one or more said Ang-2 binding molecules, further comprising one or more additional therapeutic agents, such as chemotherapeutic agents like DNA damaging agents and/or anti-mitotic drugs in cancer cells (e.g. taxol) or therapeutically active compounds that inhibit angiogenesis (an anti-angiogenic drug such as anti VEGF/VEGF receptor inhibitor, e.g. avastin, nitedanib and sunitinib), or signal transduction pathway inhibitors such as mTOR inhibitors (e.g. temsirolimus) or hormonal therapy agents (e.g. tamoxifen).

The additional therapeutic agent may be administered simultaneously with, optionally as a component of the same pharmaceutical preparation, or before or after administration of the monospecific binding molecule.

In certain embodiments, the additional therapeutic agent may be, without limitation (and in the case of the receptors, including the respective ligands), one or more inhibitors selected from the group of inhibitors of EGFR, VEGF, VEGFR, HER2-neu, Her3, AuroraA, AuroraB, PLK and PI3 kinase, FGFR, PDGFR, Raf, KSP, PDK1, PTK2, IGF-R or IR.

Further examples of additional therapeutic agents are inhibitors of CDK, Akt, src/bcr abl, cKit, cMet/HGF, c-Myc, Flt3, HSP90, hedgehog antagonists, inhibitors of JAK/STAT, Mek, mTor, NFkappaB, the proteasome, Rho, an inhibitor of wnt signaling or an inhibitor of the ubiquitination pathway or another inhibitor of the Notch signaling pathway.

Examples for Aurora inhibitors are, without limitation, PHA-739358, AZD-1152, AT 9283, CYC-116, R-763, VX-680, VX-667, MLN-8045, PF-3814735.

An example for a PLK inhibitor is GSK-461364.

Examples for VEGF inhibitor are avastin (Roche), aflibercept (Regeneron,)

Examples for raf inhibitors are BAY-73-4506 (also a VEGFR inhibitor), PLX 4032, RAF-265 (also in addition a VEGFR inhibitor), sorafenib (also in addition a VEGFR inhibitor), and XL 281.

Examples for KSP inhibitors are ispinesib, ARRY-520, AZD-4877, CK-1122697, GSK 246053A, GSK-923295, MK-0731, and SB-743921.

Examples for a src and/or bcr-abl inhibitors are dasatinib, AZD-0530, bosutinib, XL 228 (also an IGF-1R inhibitor), nilotinib (also a PDGFR and cKit inhibitor), imatinib (also a cKit inhibitor), and NS-187.

An example for a PDK1 inhibitor is BX-517.

An example for a Rho inhibitor is BA-210.

Examples for PI3 kinase inhibitors are PX-866, BEZ-235 (also an mTor inhibitor), XL 418 (also an Akt inhibitor), XL-147, and XL 765 (also an mTor inhibitor).

Examples for inhibitors of cMet or HGF are XL-184 (also an inhibitor of VEGFR, cKit, Flt3), PF-2341066, MK-2461, XL-880 (also an inhibitor of VEGFR), MGCD-265 (also an inhibitor of VEGFR, Ron, Tie2), SU-11274, PHA-665752, AMG-102, and AV-299.

An example for a c-Myc inhibitor is CX-3543.

Examples for Flt3 inhibitors are AC-220 (also an inhibitor of cKit and PDGFR), KW 2449, lestaurtinib (also an inhibitor of VEGFR, PDGFR, PKC), TG-101348 (also an inhibitor of JAK2), XL-999 (also an inhibitor of cKit, FGFR, PDGFR and VEGFR), sunitinib (also an inhibitor of PDGFR, VEGFR and cKit), and tandutinib (also an inhibitor of PDGFR, and cKit).

Examples for HSP90 inhibitors are tanespimycin, alvespimycin, IPI-504 and CNF 2024.

Examples for JAK/STAT inhibitors are CYT-997 (also interacting with tubulin), TG 101348 (also an inhibitor of Flt3), and XL-019.

Examples for Mek inhibitors are ARRY-142886, PD-325901, AZD-8330, and XL 518.

Examples for mTor inhibitors are temsirolimus, AP-23573 (which also acts as a VEGF inhibitor), everolimus (a VEGF inhibitor in addition). XL-765 (also a PI3 kinase inhibitor), and BEZ-235 (also a PI3 kinase inhibitor).

Examples for Akt inhibitors are perifosine, GSK-690693, RX-0201, and triciribine.

Examples for cKit inhibitors are AB-1010, OSI-930 (also acts as a VEGFR inhibitor), AC-220 (also an inhibitor of Flt3 and PDGFR), tandutinib (also an inhibitor of Flt3 and PDGFR), axitinib (also an inhibitor of VEGFR and PDGFR), XL-999 (also an inhibitor of Flt3, PDGFR, VEGFR, FGFR), sunitinib (also an inhibitor of Flt3, PDGFR, VEGFR), and XL-820 (also acts as a VEGFR- and PDGFR inhibitor), imatinib (also a bcr-abl inhibitor), nilotinib (also an inhibitor of bcr-abl and PDGFR).

Examples for hedgehog antagonists are IPI-609 and CUR-61414.

Examples for CDK inhibitors are seliciclib, AT-7519, P-276, ZK-CDK (also inhibiting VEGFR2 and PDGFR), PD-332991, R-547, SNS-032, PHA-690509, and AG 024322.

Examples for proteasome inhibitors are bortezomib, carfilzomib, and NPI-0052 (also an inhibitor of NFkappaB).

An example for an NFkappaB pathway inhibitor is NPI-0052.

An example for an ubiquitination pathway inhibitor is HBX-41108.

In preferred embodiments, the additional therapeutic agent is an anti-angiogenic agent.

Examples for anti-angiogenic agents are inhibitors of the FGFR, PDGFR and VEGFR or the respective ligands (e.g VEGF inhibitors like pegaptanib or the anti-VEGF antibody bevacizumab), and thalidomides, such agents being selected from, without limitation, bevacizumab, motesanib, CDP-791, SU-14813, telatinib, KRN-951, ZK-CDK (also an inhibitor of CDK), ABT-869, BMS-690514, RAF-265, IMC-KDR, IMC-18F1, IMiDs (immunomodulatory drugs), thalidomide derivative CC-4047, lenalidomide, ENMD 0995, IMC-D11, Ki 23057, brivanib, cediranib, XL-999 (also an inhibitor of cKit and Flt3), 1B3, CP 868596, IMC 3G3, R-1530 (also an inhibitor of Flt3), sunitinib (also an inhibitor of cKit and Flt3), axitinib (also an inhibitor of cKit), vemurafenib (also known as PLX4032, RG7204 or R05185426, marketed as zelboraf) a B-Raf enzyme inhibitor, crizotinib known as an ALK (anaplastic lymphoma kinase) and ROS1 (c-ros oncogenel, receptor tyrosine kinase) inhibitor, lestaurtinib (also an inhibitor of Flt3 and PKC), vatalanib, tandutinib (also an inhibitor of Flt3 and cKit), pazopanib, GW 786034, PF-337210, IMC-1121B, AVE-0005, AG-13736, E-7080, CHIR 258, sorafenib tosylate (also an inhibitor of Raf), RAF-265 (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL 880 (also an inhibitor of cMet), XL-647 (also an inhibitor of EGFR and EphB4), XL 820 (also an inhibitor of cKit), and nilotinib (also an inhibitor of cKit and brc-abl) and nitedanib.

The additional therapeutic agent may also be selected from EGFR inhibitors; it may be a small molecule EGFR inhibitor or an anti-EGFR antibody. Examples for anti-EGFR antibodies, without limitation, are cetuximab, panitumumab, matuzumab; an example for a small molecule EGFR inhibitor is gefitinib. Another example for an EGFR modulator is the EGF fusion toxin.

Among the EGFR and Her2 inhibitors useful for combination with the bispecific binding molecule of the invention are lapatinib, gefitinib, erlotinib, cetuximab, trastuzumab, nimotuzumab, zalutumumab, vandetanib (also an inhibitor of VEGFR), pertuzumab, XL-647, HKI-272, BMS-599626 ARRY-334543, AV 412, mAB-806, BMS-690514, JNJ-26483327, AEE-788 (also an inhibitor of VEGFR), ARRY-333786, IMC-11F8, Zemab.

Other agents that may be advantageously combined in a therapy with the monospecific binding molecule of the invention are tositumumab and ibritumomab tiuxetan (two radiolabelled anti-CD20 antibodies), alemtuzumab (an anti-CD52 antibody), denosumab, (an osteoclast differentiation factor ligand inhibitor), galiximab (a CD80 antagonist), ofatumumab (a CD20 inhibitor), zanolimumab (a CD4 antagonist), SGN40 (a CD40 ligand receptor modulator), rituximab (a CD20 inhibitor) or mapatumumab (a TRAIL-1 receptor agonist) or OMP-21 M18 (D114 inhibitors).

Other chemotherapeutic drugs that may be used in combination with the bispecific binding molecule s of the present invention are selected from, but not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

Particularly preferred combination partners of the bispecific binding molecules of the present invention are VEGF antagonists, like bevacizumab (Avastin®), nitedanib, Sorafenib and Sunitinib.

According to another embodiment of the invention, there is provided a method of diagnosing a disease by
a) contacting a sample with a binding molecule of the invention as defined above, and
b) detecting binding of said binding molecule to said sample, and
c) comparing the binding detected in step (b) with a standard, wherein a difference in binding relative to said sample is diagnostic of a disease or disorder associated with VEGF- and/or Ang2-mediated effects on angiogenesis.

For this and other uses, it may be useful to further modify a monospecific binding molecule of the invention, such as by introduction of a functional group that is one part of a specific binding pair, such as the biotin-(strept)avidin binding pair. Such a functional group may be used to link the binding molecule of the invention to another protein, polypeptide or chemical compound that is bound to the other half of the binding pair, i.e. through formation of the binding pair. For example, a monospecific binding molecule of the invention may be conjugated to biotin, and linked to another protein, polypeptide, compound or carrier conjugated to avidin or streptavidin. For example, such a conjugated monospecific binding molecule of the invention may be used as a reporter, for example in a diagnostic system where a detectable signal-producing agent is conjugated to avidin or streptavidin.

The efficacy of monospecific binding molecule of the invention or polypeptides, and of compositions comprising the same, can be tested using any suitable in vitro assay, cell-based assay, in vivo assay and/or animal model known per se, or any combination thereof, depending on the specific disease or disorder of interest. Suitable assays and animal models will be clear to the skilled person, and for example include the assays described herein and used in the Examples below, e.g. a proliferation assay.

Monospecific binding molecules of the invention have undergone an extensive sequence optimization process involving affinity maturation, humanization and removal of potential posttranslational modification sites to ensure low immunogenicity potential in man and improved biophysical stability. Unexpectedly, the data show that monospecific binding molecules of the invention have properties that are superior to those of binding molecules of the prior art. Among such properties are high selectivity for Ang2 neutralization as compared to Ang1 neutralization, as can e.g. be taken from the data of FIGS. 9 to 10, 13 to 14, 16 to 19; complete inhibition of the Ang2-Tie2 interaction with high potency, as can e.g. be taken from the ELISA data of FIGS. 6, 9, 13, 16, 18 and 20 and Tables 12 to 13, 16 to 17, 20 to 22, as well as the IC50 (nM) values for VHHs in the AlphaScreen assay as shown e.g. in Table 7 (Example 7); and the affinity KD (nM) of purified VHHs on recombinant human Ang2, cyno Ang2, mouse Ang2 in Tables 8, 14, 18 and 23.

This indicates that monospecific binding molecules of the invention are promising candidates to have therapeutic efficacy in diseases and disorders associated with Ang2-mediated effects on angiogenesis, such as cancer, cancerous diseases, eye diseases and/or chronic kidney diseases.

### BRIEF DESCRIPTION OF THE FIGURES

The axes annotation in FIGS. 1 to 4, 6 to 7, 9 to 10, 12 to 14 and 16 to 20: X axes stand for OD 450 (nm) and Y axes stand for log competitor (M).

FIG. 1 (FIG. 1-1A to 1-2C): Purified VHHs blocking hAng2-hTie2 interaction (ELISA)

FIG. 2 (FIG. 2-1A to 2-2C): Purified VHHs blocking mAng2-mTie2 interaction (ELISA)

FIG. 3 (FIG. 3A to 3B): Purified VHHs blocking cAng2-cTie2 interaction (ELISA)

FIG. 4 (FIG. 4A to 4I): Purified VHHs blocking hAng1-hTie2 interaction (ELISA)

FIG. 5: Sequence alignment of affinity matured variants of VHH 28D10. The amino acid sequence is aligned to the human germline VH3/JH consensus sequence. Residues are numbered according to Kabat, CDRs are shown in bold according to AbM definition. Residues that have been substituted are underlined.

FIG. 6(FIG. 6A to 6C): Purified affinity matured variants of VHH 28D10 blocking hAng2-hTie2 interaction (ELISA)

FIG. 7(FIG. 7A to 7C): Purified affinity matured variants of VHH 28D10 blocking hAng1-hTie2 interaction (ELISA)

FIG. 8(FIG. 8A to 8B): Sequence alignment of VHH 1D01 with hVH3-JH consensus (A) and of sequence optimized variants of VHH 1D01 (B). The amino acid sequence is aligned to the human germline VH3/JH consensus sequence. Residues are numbered according to Kabat, CDRs are shown in bold according to AbM definition. Residues to be mutated to their human counterpart are underlined. Potential post-translational modification sites to be tackled are boxed.

FIG. 9 (FIG. 9-1A to 9-3B): Purified sequence optimized variants of VHH 1D01 blocking hAng2-hTie2 (10-1), mAng2-mTie2 (10-2) and cAng2-cTie2 (11-3) interaction (ELISA)

FIG. 10: Purified sequence optimized variants of VHH 1D01 blocking hAng1-hTie2 interaction (ELISA)

FIG. 11 (FIG. 11A to 11C): Sequence alignment of VHH 37F02 with hVH3-JH consensus (A), of cycle 1 (B) and of cycle 2 (C) sequence optimized variants of VHH 37F02. The amino acid sequence is aligned to the human germline VH3/JH consensus sequence. Residues are numbered according to Kabat, CDRs are shown in bold according to AbM definition. Residues to be mutated to their human counterpart are underlined. Potential post-translational modification sites to be tackled are boxed.

FIG. 12 (FIG. 12-1 to 12-3): Purified cycle 1 sequence optimized variants of VHH 37F02 blocking hAng2-hTie2 (14-1), mAng2-mTie2 (14-2) and cAng2-cTie2 (14-3) interaction (ELISA)

FIG. 13(FIG. 13-1A to 13-3): Purified cycle 2 sequence optimized variants of VHH 37F02 blocking hAng2-hTie2 (15-1), mAng2-mTie2 (15-2) and cAng2-cTie2 (15-3) interaction (ELISA)

FIG. 14: Purified cycle 2 sequence optimized variants of VHH 37F02 blocking hAng1-hTie2 interaction (ELISA)

FIG. 15 (FIG. 15A to 15D): Sequence alignment of VHH 28D10 with hVH3-JHconsensus (A), of cycle 1 sequence optimized variants (B), of cycle 2 variants (C) and of cycle 3 (D) sequence optimized variants of VHH 28D10. The amino acid sequence is aligned to the human germline VH3/JH consensus sequence. Residues are numbered according to Kabat, CDRs are shown in bold according to AbM definition. Residues to be mutated to their human counterpart are underlined. Potential post-translational modification sites to be tackled are boxed.

FIG. 16 (FIG. 16-1A to 16-3C): Purified cycle 1 sequence optimized variants of VHH 28D10 blocking hAng2-hTie2 (18-1), mAng2-mTie2 (18-2) and cAng2-cTie2 (18-3) interaction (ELISA)

FIG. 17 (FIG. 17A to 17B): Purified cycle 1 sequence optimized variants of VHH 28D10 blocking hAng1-hTie2 interaction (ELISA)

FIG. 18 (FIG. 18-1A to 18-3): Purified sequence optimized C₅₀X—S₅₃X variants of VHH 28D10 blocking hAng2-hTie2 (20-1), mAng2-mTie2 (20-2) and cAng2-cTie2 (20-3) interaction (ELISA)

FIG. 19: Purified sequence optimized C₅₀X—S₅₃X variant of VHH 28D10 blocking hAng1-hTie2 interaction (ELISA)

FIG. 20(FIG. 20-1A to 20-3C): Purified cycle 2 sequence optimized variants of VHH 28D10 blocking hAng2-hTie2 (22-1), mAng2-mTie2 (22-2) and cAng2-cTie2 (22-3) interaction (ELISA)

### EXAMPLES

### Materials and Methods

### a) Generation of HEK293H Stable Cell Lines Overexpressing Human or Mouse Tie2 Receptor

The cDNAs encoding human Tie2 (NM_000459.3; SEQ ID NO:182;), mouse Tie2 (NM_013690.2; SEQ ID NO:183) and cyno Tie2 (SEQ ID NO:184); are cloned in pcDNA3.1-neo expression vector (Invitrogen, Carlsbad, Calif., USA). To establish Human Embryonic Kidney (HEK) cells overexpressing human Tie2 or mouse Tie2, parental HEK293H cells undergo lipid mediated transfection with Fugene (Roche) with pcDNA3.1-neo-hTie2 or pcDNA3.1-neo-mTie2, respectively. For all conditions, transfectants are selected 2 days post-transfection by adding 1 mg/mL geneticin (Invitrogen, Carlsbad, Calif., USA). For human, mouse and cyno Tie2, final high expressing clones are selected by single cell sorting clones binding to PE labeled anti-human Tie2 (R&D Systems, Minneapolis, Minn., US), PE labeled anti-mouse Tie2 (eBioscience, San Diego, Calif., USA) and a 2-step goat-anti-human Tie2 (R&D Systems, Minneapolis, Minn., US) followed by PE labeled donkey-anti-goat (Jackson ImmunoResearch, West Grove, Pa., USA), respectively, using the FACSAria Cell Sorter (BD Biosciences, San Jose, Calif., USA).

### b) Generation of HEK293T Cell Lines Overexpressing Mouse or Cynomolgus Ang2 and Production of Recombinant Mouse and Cynomolgus Ang2 Conditioned Medium

The cDNAs encoding N-terminally FLAG-tagged mouse Ang2 (NM_007426.3; SEQ ID SEQ ID NO: 185) and cynomolgus Ang2 (AB172643.1; SEQ ID NO: 186) are cloned in a pSecTag2B expression vector (Invitrogen, Carlsbad, Calif., USA). Human Embryonic Kidney (HEK) cells transiently overexpressing mouse Ang2 or cynomolgus Ang2 are generated by lipid-mediated transfection (Fugene; Roche) of pSecTag2B-mAng2 or pSecTag2B-cAng2, respectively, in the HEK293T parental cell line. Productions are performed in 1.5 liter CF10 Bag, and 1.5 L conditioned medium (CM) is collected 5 days post-transfection.

### c) Production of Recombinant Cynomolgus Tie2/Fc Chimera in HEK293-F Cells

The cDNA encoding for the extracellular domain of Tie-2 is subcloned into the expression plasmid pSecTag2b using appropriate restriction sites to generate an Fc-fusion protein. Transfection into HEK293-F cells (Invitrogen) is performed as described by the manufacturer using Megaprep (Qiagen) preparations of plasmids, Optimem-Medium (Invitrogen), 293-fectin (Invitrogen) at an initial cell density of 1×10⁶ viable cells/mL with 1 μg plasmid DNA/10⁶ cells. Transfected cells are cultivated in shaker flasks for 7 days at 37° C. Conditioned Medium (CM) is harvested by centrifugation at 4000 g for 10 min and filtered through a sterile filter (0.45 μm membrane).

Fc-fusion proteins are purified using affinity chromatography by loading the CM at 5 ml/min onto a 5 ml MabSelect SuRe Protein A column equilibrated with DPBS. After a washing step with DPBS, bound Fc-protein is eluted with 10 mM sodium citrate buffer pH 3.0 and subsequently neutralized to pH 7.0 by adding 1 M Tris/HCl pH 8.0. The purified protein is concentrated and buffer exchanged to DPBS with a Millipore Amicon Ultra (10 kDa molecular weight cuttoff) centrifugal concentrator. Presence of the protein is confirmed with standard analytical methods (electrophoresis with Experion Pro 260 kit-BioRad; mass spectrometry). The protein is further analysed by size-exclusion chromatography and the endotoxin-content is determined (Endosafe PTS kit—Charles River).

### d) Production of Recombinant Human, Cynomolgus, Mouse and Rat Ang2-FLD in HEK293-F Cells

Molecular cloning and cell culture is performed as described for Tie2-Fc-fusion protein. For purification of His-tagged proteins the CM is loaded at 5 ml/min on a 2 ml Ni²⁺ chelating sepharose fast flow column (His-Trap—GE Healthcare Life Sciences) equilibrated with DPBS. After loading in the presence of 4% elution buffer (DPBS+0.5% imidazol) to prevent unspecific binding, the column is washed with DPBS. Ang2-FLD-proteins are eluted from the column with DPBS containing 0.5% imidazol. Subsequently an ultrafiltration step was done for concentration and buffer exchange (10 kDa molecular withgt cut off). An aliquot of the protein is retained for analytical characterization as described for Tie2-Fc.

### Example 1

Immunization with Recombinant Human Ang2 Induces a Humoral Immune Response in Llama

### 1.1. Immunizations

After approval of the Ethical Committee of the faculty of Veterinary Medicine (University Ghent, Belgium), 4 llamas (designated No. 406, 408, 454, 455) are immunized with 4 intramuscular injections (day 0: 50 μg, day 14: 20 μg, day 28: 17.5 μg and day 42: 17.5 μg dose) of recombinant human Ang2 (R&D Systems, Minneapolis, Minn., US). The antigen is formulated in Complete Freund's Adjuvant for the prime injection at day 0 (Difco, Detroit, Mich., USA) and in Incomplete Freund's Adjuvant for the booster injections (Difco, Detroit, Mich., USA).

### 1.2. Evaluation of Induced Immune Responses in Llama

To evaluate the induction of an immune response in the animals against human Ang2 by ELISA, sera are collected at day 0 (pre-immune), day 35 and day 46 (time of peripheral blood lymphocyte [PBL] collection). In short, 1 μg/mL of recombinant human Ang2 (R&D Systems, Minneapolis, Minn., USA) is immobilized overnight at 4° C. in a 96-well MaxiSorp plate (Nunc, Wiesbaden, Germany). Wells are blocked with a casein solution (PBS+1% casein). After addition of serial serum dilutions, specifically bound immunoglobulins are detected using a horseradish peroxidase (HRP)-conjugated goat anti-llama immunoglobulin (Bethyl Laboratories Inc., Montgomery, Tex., USA) and a subsequent enzymatic reaction in the presence of the substrate TMB (3,3′,5,5′-tetramentylbenzidine) (Pierce, Rockford, Ill., USA), showing that a significant antibody dependent immune response against human Ang2 is induced. The antibody response is mounted both by conventional and heavy-chain only antibody expressing B-cell repertoires, since bound immunoglobulins can be detected with antibodies specifically recognizing the conventional llama IgG1 antibodies or the heavy chain only llama IgG2 or IgG3 antibodies. In all llamas injected with human Ang2, an antibody response is mounted by conventional and heavy chain only antibody expressing B-cells specifically against human Ang2. The Ang2 serum titer responses for each llama are depicted in Table 1.

TABLE 1
Antibody mediated specific serum response
against recombinant human Ang2.

IgG reponse

| Llama | Immunogen | IgG1 | IgG2 | IgG3 |
|---|---|---|---|---|
| 406 | rec. human Ang2 | +++ | ++ | ++ |
| 408 | rec. human Ang2 | +++ | ++ | ++ |
| 454 | rec. human Ang2 | +++ | ++ | ++ |
| 455 | rec. human Ang2 | +++ | ++ | +++ |

Legend: (*)
Low (or +/−): 1,000 ≥ HSD_{S/N≥2} < 1,500
Moderate (or +): 1,500 ≥ HSD_{S/N≥2} < 13,500
Good (or ++): 13,500 ≥ HSD_{S/N≥2} < 365,000
Excellent (or +++): HSD_{S/N≥2} ≥ 365,000
(*) HSD, Highest Serum Dilution; S/N ≥ 2, signal-to-noise ratio ≥ 2

### Example 2

### Cloning of the Heavy-Chain Only Antibody Fragment Repertoires and Preparation of Phage

Following the final immunogen injection, immune tissues as the source of B-cells that produce the heavy-chain only antibodies are collected from the immunized llamas. Typically, two 150 mL blood samples collected 4 and 10 days after the last antigen injection, and one lymph node biopsy, collected 4 days after the last antigen injection are collected per animal. From the blood samples, peripheral blood mononuclear cells (PBMCs) are prepared using Ficoll-Hypaque according to the manufacturer's instructions (Amersham Biosciences, Piscataway, N.J., USA). From the PBMCs and the lymph node biopsy (not prelevated from animal No. 406), total RNA is extracted, which is used as starting material for RT-PCR to amplify the VHH encoding DNA segments, as described in Example 3 (page 46) of WO 05/044858. For each immunized llama, a library is constructed by pooling the total RNA isolated from all collected immune tissues of that animal. In short, the PCR-amplified VHH repertoire is cloned via specific restriction sites into a vector designed to facilitate phage display of the VHH library. The vector is derived from pUC119 and contains the LacZ promoter, a M13 phage gill protein coding sequence, a resistance gene for ampicillin or carbenicillin, a multiple cloning site and a hybrid gIII-peIB leader sequence. In frame with the VHH coding sequence, the vector encodes a C-terminal c-myc tag and a His6 tag. Phage are prepared according to standard protocols and stored after filter sterilization at 4° C. for further use.

### Example 3

### Selection of Ang2 Specific VHHs Via Phage Display

VHH repertoires obtained from all llamas and cloned as phage library are used in different selection strategies, applying a multiplicity of selection conditions. Variables include i) the Ang2 protein format: biotinylated C-terminally His-tagged full length recombinant human Ang2 (R&D Systems, Minneapolis, Minn., USA) and C-terminally His-tagged full length mouse Ang2 (produced at GeneArt, now Invitrogen, Carlsbad, Calif., USA), ii) the Ang2 presentation method: plates directly coated with mouse Ang2 or incubation in solution with biotinylated human Ang2 followed by capturing on neutravidin-coated plates, and iii) the antigen concentration. All selections are done in 96 well MaxiSorp plates (Nunc, Wiesbaden, Germany).

Multi-round selections are performed as follows: Ang2 preparations for solid and solution phase selection formats are presented as described above at multiple concentrations (biotinylated human Ang2: 50, 5, 0.5, 0.05 and 0.005 nM; mouse Ang2: 10, 1, 0.1 and 0.01 μg/mL). After 2 h incubation with the phage libraries, followed by extensive washing, bound phages are eluted with trypsin (1 mg/mL) for 15-30 minutes at room temperature. Trypsin activity is then immediately neutralized by applying 0.8 mM protease inhibitor ABSF. As background control, selections w/o antigen are performed in parallel. Phage outputs that show enrichment over background are used to infect E. coli. Infected E. coli cells are either used to prepare phage for the next selection round (phage rescue) or plated on LB agar plates (ampicillin+glucose^{2%}) for analysis of individual VHH clones. In order to screen a selection output for specific binders or blockers, single colonies are picked from the agar plates and grown in 1 mL 96-deep-well plates. LacZ-controlled VHH expression is induced by adding IPTG (0.1-1 mM final) in the absence of glucose. Periplasmic extracts (in a volume of ˜80 uL) are prepared according to standard protocols (as disclosed in for example WO 2006/040153 cited herein). Briefly, cultures were centrifuged for 15 minutes at 4,500 rpm. The pellet was frozen overnight or for 1 hour at −20° C. Next, the pellet was thawed at room temperature for 40 minutes, re-suspended in 15 ml peri buffer (50 mM NaHPO4, 300 mM NaCl) and shaken for 1 hour. Periplasmic fraction was isolated by centrifugation for 20 minutes at 14000 rpm.

### Example 4

### Screening of Periplasmic Extracts in Ang2-Tie2 and Ang1-Tie2 ELISA and AlphaScreen Competition Assays

Periplasmic extracts containing expressed VHHs are screened in a human Ang2-human Tie2 AlphaScreen competition assay to assess their blocking capacity. In brief, human Tie2/Fc chimera (R&D Systems, Minneapolis, Minn., USA) is biotinylated using N-hydroxysulfosuccinimide ester of biotin (Thermo Fisher Scientific, Rockford, Ill., USA). FLAG tagged human Ang2 (Alexis Biochemicals, San Diego, Calif., USA) is captured using Acceptor beads (Perkin Elmer, Waltham, Mass., US) coated with anti-FLAG M2 antibody (Sigma, St Louis, Mo., USA). To evaluate the capacity of the VHHs to inhibit binding of human Ang2 to its receptor human Tie2, 1:25 dilutions of the periplasmic extracts containing expressed VHHs are incubated with 0.1 nM FLAG tagged human Ang2. To this mixture, the Acceptor beads and 0.3 nM biotinylated human Tie2/Fc chimeras are added and further incubated for 2 hours at room temperature. Finally, streptavidin conjugated Donor beads (Perkin Elmer, Waltham, Mass., US) are added and the mixture is incubated for an additional 2 hours at room temperature. Assay buffer is PBS+0.03% Tween-20+0.1% BSA. Fluorescence is measured using the Envision Multilabel Plate reader (Perkin Elmer, Waltham, Mass., USA) using an excitation wavelength of 680 nm and an emission wavelength of 520 nm. Decrease in fluorescence signal indicates that the binding of human Ang2 to human Tie2 is blocked by the VHH expressed in the periplasmic extract. VHHs able to block the human Ang2-human Tie2 interaction for at least 50% are screened in a confirmatory ELISA based competition assay. Additionally, cross-reactivity for binding to mouse Ang2 and selectivity over human Ang1 is also assessed in a competition ELISA. In brief, human or mouse Tie2/Fc chimera (R&D Systems, Minneapolis, Minn., USA) are immobilized at 2 μg/mL overnight at 4° C. in a 96-well MaxiSorp plate (Nunc, Wiesbaden, Germany). Wells are blocked with a 1% casein solution. A 1:5 dilution of periplasmic extract containing expressed VHHs is incubated with the following Ang species according to the type of assay: 0.02 nM FLAG tagged human Ang2, a 1:3,000 dilution of HEK293 conditioned medium containing FLAG tagged mouse Ang2 or 0.02 nM FLAG-tagged human Ang1 (Alexis Biochemicals, San Diego, Calif., USA). This mixture is added to the Tie2/Fc coated well and incubated for 2 hours at room temperature. Residual binding of Ang is detected using HRP-conjugated anti-FLAG M2 antibody (Sigma, St Louis, Mo., USA).

In a second screening cycle, periplasmic extracts containing expressed VHHs of selection outputs that yielded a high diversity of mouse Ang2 cross-reactive blocking VHHs are screened at a 1:300 dilution. VHHs inhibiting the binding of human Ang2 to human Tie2, mouse Ang2 to mouse Tie2 and showing no inhibition of human Ang1 binding to human Tie2 are selected. Sequence analysis revealed 86 unique VHHs belonging to 38 different B-cell lineages. The total number of unique sequence variants found for each B-cell lineage, a representative VHH and the selection condition used, is depicted in Table 2. An overview of AlphaScreen and ELISA based screening data is given in Table 3. The amino acid sequences of all unique VHHs are shown in the Sequence Listing (SEQ ID NOs: 1 to 86) and in Table 4.

TABLE 2
Selection parameters used for the identification
of Ang2 specific VHH B-cell lineages.

| B-cell | Representative | # Unique | | | Selection |
|---|---|---|---|---|---|
| Lineage no. | VHH ID | variants | Library | Selection format | rounds |
| 1 | 2F04 | 3 | 408 | biot-hAng2 | 1 or 2 |
| | | | | biot-hAng2 > biot-hAng2 | |
| | | | | rmAng2 > rmAng2 | |
| 2 | 1D01 | 2 | 406 | biot-hAng2 | 1 or 2 |
| | | | | biot-hAng2 > rmAng2 | |
| 3 | 10H02 | 7 | 408 | biot-hAng2 > biot-hAng2 | 1 or 2 |
| | | | | rmAng2 > rmAng2 | |
| 4 | 3A07 | 3 | 454 | biot-hAng2 | 1 or 2 |
| | | | | biot-hAng2 > rmAng2 | |
| 5 | 7G08 | 3 | 454 | biot-hAng2 > biot-hAng2 | 1 or 2 |
| 6 | 2G01 | 1 | 408 | biot-hAng2 | 1 |
| 7 | 8A11 | 1 | 455 | biot-hAng2 > biot-hAng2 | 2 |
| 8 | 16A03 | 4 | 455 | biot-hAng2 | 1 or 2 |
| | | | | biot-hAng2 > rmAng2 | |
| 9 | 14A09 | 2 | 408 | biot-hAng2 | 1 or 2 |
| | | | | biot-hAng2 > rmAng2 | |
| 10 | 11B07 | 18 | 454 | biot-hAng2 | 1 or 2 |
| | | | | biot-hAng2 > biot-hAng2 | |
| | | | | biot-hAng2 > rmAng2 | |
| | | | | rmAng2 > rmAng2 | |
| 11 | 1E01 | 1 | 406 | biot-hAng2 | 1 |
| 12 | 13A03 | 2 | 406 | biot-hAng2 > rmAng2 | 2 |
| 13 | 15A06 | 1 | 454 | biot-hAng2 > rmAng2 | 2 |
| 14 | 11A03 | 2 | 454 | rmAng2 > rmAng2 | 2 |
| 15 | 14H02 | 3 | 408 | biot-hAng2 > rmAng2 | 2 |
| | | | | rmAng2 > rmAng2 | |
| 16 | 14A08 | 1 | 408 | biot-hAng2 > rmAng2 | 2 |
| 17 | 15H04 | 1 | 454 | biot-hAng2 > rmAng2 | 2 |
| 18 | 16G09 | 3 | 455 | biot-hAng2 > rmAng2 | 2 |
| 19 | 13A02 | 1 | 406 | biot-hAng2 > rmAng2 | 2 |
| 20 | 10C06 | 4 | 408 | rmAng2 > rmAng2 | 2 |
| 21 | 12A08 | 1 | 455 | rmAng2 > rmAng2 | 2 |
| 22 | 12B03 | 2 | 455 | biot-hAng2 > rmAng2 | 2 |
| | | | | rmAng2 > rmAng2 | |
| 23 | 10A03 | 1 | 408 | rmAng2 > rmAng2 | 2 |
| 24 | 16A02 | 2 | 455 | biot-hAng2 > rmAng2 | 2 |
| | | | | rmAng2 > rmAng2 | |
| 25 | 10A09 | 3 | 408 | biot-hAng2 > rmAng2 | 2 |
| | | | | rmAng2 > rmAng2 | |
| 26 | 22C07 | 1 | 408 | rmAng2 > rmAng2 | 2 |
| 27 | 21G10 | 2 | 408 | biot-hAng2 > rmAng2 | 2 |
| | | | | rmAng2 > rmAng2 | |
| 28 | 19A03 | 1 | 406 | biot-hAng2 > rmAng2 | 2 |
| 29 | 23C10 | 1 | 454 | biot-hAng2 > rmAng2 | 2 |
| 30 | 25B01 | 1 | 455 | biot-hAng2 > rmAng2 | 2 |
| 31 | 25F01 | 1 | 455 | biot-hAng2 > rmAng2 | 2 |
| 32 | 25D08 | 1 | 455 | biot-hAng2 > rmAng2 | 2 |
| 33 | 24B05 | 1 | 454 | rmAng2 > rmAng2 | 2 |
| 34 | 22G11 | 1 | 408 | rmAng2 > rmAng2 | 2 |
| 35 | 25G04 | 1 | 455 | biot-hAng2 > rmAng2 | 2 |
| 36 | 28D10 | 1 | 408 | biot-hAng2 > rmAng2 | 2 |
| 37 | 32H10 | 1 | 408 | rmAng2 > rmAng2 | 2 |
| 38 | 29B08 | 1 | 408 | biot-hAng2 > rmAng2 | 2 |

TABLE 3
Screening of periplasmic extracts containing expressed
anti-Ang2 VHH^{(a)}

| B-cell | Represen- | # | AlphaScreen | ELISA | | |
|---|---|---|---|---|---|---|
| Lineage | tative | Unique | hAng2 | hAng2 | mAng2 | hAng1 |
| No. | VHH ID | variants | (% inh) | (% inh) | (% inh) | (% inh) |
| 1 | 2F04 | 3 | 79-84 | 50-86 | 13-27 | 0-4 |
| 2 | 1D01 | 2 | 77-83 | 97-101 | 85-102 | 0 |
| 3 | 10H02 | 7 | 54-94 | 65-101 | 42-98 | 0-3 |
| 4 | 3A07 | 3 | 53-80 | 0-75 | 1-27 | 0-4 |
| 5 | 7G08 | 3 | 96-96 | 101 | 101 | 0 |
| 6 | 2G01 | 1 | 55 | 39 | 2 | 0 |
| 7 | 8A11 | 1 | 57 | 67 | 13 | 0 |
| 8 | 16A03 | 4 | 54-96 | 0 | 0-4 | 0-2 |
| 9 | 14A09 | 2 | 0-69 | 6-13 | 0-2 | 0 |
| 10 | 11B07 | 18 | 46-92 | 26-107 | 9-93 | 0-2 |
| 11 | 1E01 | 1 | 0 | 2 | 3 | 4 |
| 12 | 13A03 | 2 | 50-53 | 28-40 | 0 | 0-2 |
| 13 | 15A06 | 1 | 72 | 0 | 0 | 0 |
| 14 | 11A03 | 2 | 57-74 | 0-3 | 0 | 0 |
| 15 | 14H02 | 3 | 54-63 | 54-56 | 36-48 | 0-7 |
| 16 | 14A08 | 1 | 52 | 5-5 | 4 | 0 |
| 17 | 15H04 | 1 | 57 | 82 | 38 | 0 |
| 18 | 16G09 | 3 | 55-95 | 57-99 | 21-96 | 1-7 |
| 19 | 13A02 | 1 | 91 | 96 | 97 | 1 |
| 20 | 10C06 | 4 | 64-85 | 84-90 | 43-50 | 0-2 |
| 21 | 12A08 | 1 | 98 | 0 | 2 | 3 |
| 22 | 12B03 | 2 | 83-92 | 92-94 | 11-99 | 0-7 |
| 23 | 10A03 | 1 | 67 | 0 | 0 | 0 |
| 24 | 16A02 | 2 | 67-90 | 0 | 0-1 | 0-1 |
| 25 | 10A09 | 3 | 52-65 | 0-11 | 0-1 | 0-4 |
| 26 | 22C07 | 1 | 73 | 78 | 82 | 0 |
| 27 | 21G10 | 2 | 55-85 | 45-72 | 13-30 | 0 |
| 28 | 19A03 | 1 | 74 | 74 | 22 | 0 |
| 29 | 23C10 | 1 | 57 | 37 | 24 | 0 |
| 30 | 25B01 | 1 | 61 | 5 | 24 | 4 |
| 31 | 25F01 | 1 | 67 | 85 | 42 | 14 |
| 32 | 25D08 | 1 | 78 | 94 | 6 | 10 |
| 33 | 24B05 | 1 | 97 | 102 | 98 | 3 |
| 34 | 22G11 | 1 | 96 | 100 | 98 | 0 |
| 35 | 25G04 | 1 | 90 | 87 | 9 | 0 |
| 36 | 28D10 | 1 | 89 | 118 | 93 | 4 |
| 37 | 32H10 | 1 | 55 | 112 | 58 | 17 |
| 38 | 29B08 | 1 | 60 | 112 | 63 | 0 |

^{(a)}if multiple unique VHH variants within a B-cell lineage are identified, the range (min-max) of % inhibition is given.

TABLE 4
Amino acid sequence of unique anti-Ang2 VHHs identified
during screening

| VHH ID/ | | | | | | | |
|---|---|---|---|---|---|---|---|
| SEQ ID | | | | | | | |
| NOs: | FR1 | CDR1 | FR2 | CDR2 | FR3 | CDR3 | FR4 |
| 001D01/1 | EVQLVES | DYAL | WFRQA | CIRCSD | RFTISSDNAK | SIVPRSKLE | WGQ |
| | GGGLVQA | G | AGKERE | GSTYYA | NTVYLQMNSL | PYEYDA | GTQV |
| | GGSLRLS | | GVS | DSVKG | KPEDTAVYYC | | TVSS |
| | CAASGFT | | | | AA | | |
| | FD | | | | | | |
| 001E01/2 | EVQLVES | THAM | WYRQA | TFTNRG | RFTISRDNAK | GPY | WGQ |
| | GGGLVQP | G | PGKQR | STYYAG | NTMYLQMNSL | | GTQV |
| | GGSLRLS | | EHVA | SVKG | KPEDTAVYYC | | TVSS |
| | CVHSGTIS | | | | NT | | |
| | S | | | | | | |
| 002A01/3 | EVQLVES | SSVM | WFRQA | AISGSG | RFTISRDNAK | GRAFLARDT | WGQ |
| | GGGLVQA | G | PGKERE | SSTDSA | NTVYLQMNSL | FYYDI | GTQV |
| | GGSLRLS | | FVA | QG | KPEDTAVYYC | | TVSS |
| | CAASGRT | | | | AA | | |
| | FS | | | | | | |
| 002F04/4 | EVQLVES | DHLI | WFRQA | CISRSA | RFTISSDNAK | GPAWGRPA | WGQ |
| | GGGLVQA | G | PGKERE | GSTYYA | NTVYLQMNSL | SPLPYEYDY | GTQV |
| | GGSLRLS | | AVS | DSVKG | KADDTAVYYC | | TVSS |
| | CAASGFA | | | | AA | | |
| | FD | | | | | | |
| 002G01/5 | EVQLVES | SYAM | WFRQA | AISLSG | RFTISRDNAK | TDWDFEDIP | WGQ |
| | GGGLVQA | A | PGKERE | DSTYYA | NTVYLQMNSL | EYYCSGYG | GTQV |
| | GGSLRLS | | FVA | DSVKG | KPEDTAEYYC | CDESLFDS | TVSS |
| | CAASGRTI | | | | AA | | |
| | S | | | | | | |
| 003A07/6 | EVQLVES | YDAI | WFRQA | CITSSD | RFTISRDNAK | GNRRIYYSD | WGQ |
| | GGGLVQP | G | PGKERE | GITYYA | NTVYLQMNSL | YALACFPYE | GTQV |
| | GGSLRLS | | GVS | DSVKG | KPEDTAVYYC | YDY | TVSS |
| | CAASGFT | | | | AT | | |
| | LD | | | | | | |
| 003D01/7 | EVQLVES | TYLM | WFRQA | VIWSSG | RFTISRDSAK | SYDGNYYIP | WGQ |
| | GGGLVQV | VG | PGKERE | DTAYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |
| 003E10/8 | EVQLVES | YYAV | WFRQA | CISSSD | RFTISRDNAK | GNLRIYYSD | WGQ |
| | GGGLVQP | G | PGKERE | GSTYYA | NTVYLQMNSL | YALACFPYE | GTQV |
| | GGSLRLS | | GVS | DSVKG | KPEDTAVYYC | YDY | TVSS |
| | CTTSGFTL | | | | AT | | |
| | D | | | | | | |
| 003F02/9 | EVQLVES | TYLM | WFRQA | GIWSSG | RFTISRDNAK | SYDGNYYIP | WGQ |
| | GGGLVQV | VG | PGKERE | DTAYAD | NTVYLQMNSL | GFYKD | GTQV |
| | GDSLRLA | | FAA | SVKG | KTEDTAVYYC | | TVSS |
| | CAASGRT | | | | AG | | |
| | FS | | | | | | |
| 003F07/10 | EVQLVES | TYLM | WFRQA | AMWSS | RFTISRDNAK | SYGGNYYIP | WGQ |
| | GGGLVQV | VG | PGKERE | GVPAYA | NTVYLQMNSL | GFYED | GTQV |
| | GDSLRLS | | FAA | DSVKG | KAEDTAVYYC | | TVSS |
| | CAASGRT | | | | AG | | |
| | FS | | | | | | |
| 004B06/11 | EVQLVES | RYAM | WFRRV | HITWNR | RFTISRDKAS | QIKYGAVTH | WGQ |
| | GGGLVQA | G | PGKERE | GSTYYA | NTLYLQMNSL | PEEYSY | GTQV |
| | GGSLRLS | | FVT | DSVKG | KPEDTAVYYC | | TVSS |
| | CAASGHT | | | | AA | | |
| | FS | | | | | | |
| 006F05/12 | KVQLVES | DHLI | WFRQA | CISRSA | RFTISSDNAK | GPAWGRPA | WGQ |
| | GGGLVQA | G | PGKERE | GSTYYA | NTVYLQMNSL | SPLPYEYDY | GTQV |
| | GGSLRLS | | AVS | DSVKG | KADDTAVYYC | | TVSS |
| | CAASGFA | | | | AA | | |
| | FD | | | | | | |
| 006H05/13 | EVQLVES | DYAI | WFRQA | CISSSD | RFTISSDNAK | VPATRRTPQ | WGQ |
| | GGGLVQA | G | PGKERE | GSTAYA | NTVYLQMNSL | MVAANVCW | GTQV |
| | GGSLRLS | | GVS | DSVKG | KPEDTAVYYC | LAEYEYDY | TVSS |
| | CAASGFT | | | | TA | | |
| | FD | | | | | | |
| 007B09/14 | EVQLVES | TYLM | WFRQA | GIWSSG | RFTVSRDNAK | SYDGNYYIP | WGQ |
| | GGGLVQV | VG | PGKERE | GTAYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |
| 007C01/15 | EVQLVEF | TYLM | WFRQA | GIWSSG | RFTVSRDNDK | SYDGNYYIP | WGQ |
| | GGGLVQV | VG | PGKERE | GTAYAD | NTVYLQMNSL | GFYKD | GTQV |
| | GDSLRLS | | FAA | SVKE | QAEDTAVYYC | | TVSS |
| | CAASGRT | | | | AG | | |
| | FS | | | | | | |
| 007C07/16 | EVQLVES | TYLM | WFRQT | VIWSSG | RFTISRDNAK | SYGGNYYIP | WGQ |
| | GGGLVQV | VG | PGKERE | DTAYAD | NTVYLQMNSL | GFYED | GTQV |
| | GDSLRLS | | FAA | SVKG | KAEDTAVYYC | | TVSS |
| | CAASGHT | | | | AG | | |
| | FS | | | | | | |
| 007G08/17 | EVQLVES | YYAI | WFRQV | CISSSD | RFTISRDNAK | DSGGYIDYD | WGQ |
| | GGGLVQP | G | PGKERE | GITYYV | NTVYLQMNSL | CMGLGYDY | GTQV |
| | GGSLRLS | | GVS | DSVKG | KPEDTAVYYC | | TVSS |
| | CAASGFA | | | | AT | | |
| | LD | | | | | | |
| 008A11/18 | EVQLVES | SYAM | WFRQA | AVSWS | RFTISRDSAK | QSTIVEVTTL | WGQ |
| | GGGLVQA | G | PGKELE | GGSTYY | NTVYLQMNSL | EAYDY | GTQV |
| | GGSLRLS | | FVT | ADSVKG | KPEDTAVYYC | | TVSS |
| | CAASGRT | | | | AA | | |
| | FS | | | | | | |
| 010A03/19 | EVQLVES | PYAM | WFRQA | HITWSA | RFTISRDNAK | KRRYGIVDR | WGQ |
| | GGGSVQA | G | PGKERE | GSTYYA | NTVYLQMNSL | DYND | GTQV |
| | GGSLRLS | | FVA | DSVKG | KPEDTAVYYC | | TVSS |
| | CAASERT | | | | AA | | |
| | FS | | | | | | |
| 010A09/20 | EVQLVES | SVSA | WYRQA | GISNIGA | RFTISRDNAK | LLWSGNL | WGQ |
| | GGGLVQA | TG | PGKQR | TKFADS | NTVYLQMNSL | | GTQV |
| | GGSLRLA | | EFVA | VKG | KPEDTAVYYC | | TVSS |
| | CIASGRDI | | | | NV | | |
| | F | | | | | | |
| 010A10/21 | EVQLVES | SITAI | WYRQA | GISNIGA | RFTISGDNAE | LLWSANY | WGQ |
| | GGGLVQA | G | PGKQR | TKYTDS | NTVYLQMNSL | | GTQV |
| | GGSLRLS | | EFVA | VKG | KPEDTAVYYC | | TVSS |
| | CIASGRDI | | | | NV | | |
| | F | | | | | | |
| 010B02/22 | EVQLVES | DHLI | WFRQA | CISRSA | RFTISGDNAK | GPAWGRPA | WGQ |
| | GGGLVQA | G | PGKERE | GSTYYA | NTVYLQMNSL | SPLPYEYDY | GTQV |
| | GGSLRLS | | AVS | DSVKG | KADDTAVYYC | | TVSS |
| | CAASGFA | | | | AA | | |
| | FD | | | | | | |
| 010B08/23 | EVQLVES | DYAI | WFRQA | CISSSD | RFTISSDNAK | VPATRRTPQ | WGQ |
| | GGGLVQA | G | PGKERE | GSTAYA | NTVYLQMNSL | MVVANVCW | GTQV |
| | GGSLRLS | | GVS | DSVKG | KPEDTAVYYC | LAEYEYDY | TVSS |
| | CAASGFT | | | | TA | | |
| | FD | | | | | | |
| 010C06/24 | EVQLVES | DYAI | WFRQA | CISSSD | RFTISGDNAK | GITPCSDYT | WGQ |
| | GGGLVQA | G | PGKERE | GSTYYA | NTVYLQMNSL | QTYEYDV | GTQV |
| | GGSLRLS | | GVS | DSVKG | KPEDTAVYYC | | TVSS |
| | CVASGFT | | | | AA | | |
| | FD | | | | | | |
| 010C07/25 | EVQLVES | DYAI | WFRQA | CISRSD | RFTISSDNAK | VPATRRTPQ | WGQ |
| | GGGLVQA | G | PGKERE | GSTSYA | NTVYLVMNSL | MVVANMCW | GTQV |
| | GGSLRLS | | GVS | DSVKG | KPEDTAVYYC | LAEYEYDY | TVSS |
| | CAASGFT | | | | TA | | |
| | FD | | | | | | |
| 010D04/26 | EVQLVES | DYAI | WFRQA | CISSSD | RFTISSDNAK | GITPCSDYT | WGQ |
| | GGGLVQA | G | PGKERE | GSTYYA | NTVYLQMNSL | QTYEYDV | GTQV |
| | GGSLRLS | | GVS | DSVKG | KPEDTAVYYC | | TVSS |
| | CAASGFT | | | | AA | | |
| | FD | | | | | | |
| 010E02/27 | EVQLVES | DYAI | WFRQA | CISSSD | RFTISSDNAK | VPATRRTPQ | WGQ |
| | GGGLVQA | G | PGKERE | GSTAYA | NTVYLQMNSL | MVVANVCW | GTQV |
| | GGSLRLS | | GVS | DSVKG | KPEDTAVYYC | LAEYEYDY | TVSS |
| | CAVSGFT | | | | TA | | |
| | FD | | | | | | |
| 010F10/28 | EVQLVES | DYAI | WFRQA | YISSSD | RFTSSSDNAK | RPTLRVRLD | WGQ |
| | GGGLVQA | G | PGKERE | GSTYYA | NTVYLQMNSL | NDRHHLLYE | GTQV |
| | GGSLRLS | | GVS | DSVKG | KPEDTAVYYC | YEYDY | TVSS |
| | CAASGFT | | | | AA | | |
| | FD | | | | | | |
| 010G02/29 | EVQLVES | DYTI | WFRQA | CISSSD | RFTISSDNAK | VPATRRTPQ | WGQ |
| | GGGLVQA | G | PGKERE | GSTSYA | NTVYLQMNSL | MVVLNMCW | GTQV |
| | GGSLRLS | | GVS | DSVKG | KPEDTAVYYC | LAEYEYDY | TVSS |
| | CAASGFT | | | | TA | | |
| | FD | | | | | | |
| 010G11/30 | EVQLVES | DYAI | WFRQA | CISSSD | RFTISSDNAK | GITPCSDYT | WGQ |
| | GGGLVQA | G | PGKERE | GSTYYA | NTVYLQMNSL | QTYEYDV | GTQV |
| | GGSLRLS | | GVS | DSVKG | KPEDTAVYYC | | TVSS |
| | CVASGFT | | | | AA | | |
| | FD | | | | | | |
| 010H02/31 | EVQLVES | DYAI | WFRQA | CISSSD | RFTISSDTAKN | VPATRRTPQ | WGQ |
| | GGGSVQA | G | PGKERE | GSTNYA | TVYLQMNSLK | MVDANMCW | GTQV |
| | GGSLRLS | | GVS | DSVKG | PEDTAVYYCT | LAEYEYDY | TVSS |
| | CAASGFT | | | | A | | |
| | FD | | | | | | |
| 011A02/32 | EVQLVES | GHA | WFRQA | TIYWTS | RFTISRDNAE | IKDFQLRVD | WGQ |
| | GGGLAQA | MG | PGEERE | GMTRYA | NTVFLQMNSL | VTSASAYDY | GTQV |
| | GGSLRLS | | FVA | GSVKG | KPEDTAVYYC | | TVSS |
| | CAASGRR | | | | AV | | |
| | FG | | | | | | |
| 011A03/33 | GVQLVES | GHA | WFRQA | TIYWTT | RFTISRDNAE | IRDFNIRLDV | WGQ |
| | GGGLAQA | MG | PGKDRE | GMTRYA | NTVFLQMNSL | TSASAYGY | GTQV |
| | GGSLRLS | | FVA | DSVKG | KPEDTAVYYC | | TVSS |
| | CAASGRR | | | | AL | | |
| | FG | | | | | | |
| 011B07/34 | EVQLVES | TYLM | WFRQA | GIWSSG | RFTISRDNAK | SYDGNYYIP | WGQ |
| | GGGLVQV | VG | PGKERE | DTAYAD | NTVYLQMNSL | GFYKD | GTQV |
| | GDSLRLS | | FAA | SVRG | KTEDTAVYYC | | TVSS |
| | CAASGRT | | | | AG | | |
| | FS | | | | | | |
| 011C01/35 | EVQLVES | TYLV | WFRQA | AIWSSG | RFTISRDNAK | SYGGNYYIP | WGQ |
| | GGGLVQV | VG | PGKERE | DTAYAD | NTVYLQMNSL | GFYED | GTQV |
| | GDSLRLS | | FAA | SVKG | KAEDTAVYYC | | TVSS |
| | CAASGRT | | | | AG | | |
| | FS | | | | | | |
| 012A02/36 | EVQLVES | RNA | WFRQV | GIRWNV | RFTISRDNAE | YAGLVFSGI | WGQ |
| | GGGLVQA | MA | PGKVRE | GRLDYA | NTVYLQMNDL | PDY | GTQV |
| | GGSLRLS | | FVA | DSVKG | KTEDTAVYYC | | TVSS |
| | CAASGRT | | | | AA | | |
| | FS | | | | | | |
| 012A08/37 | EVQLVES | INAM | WYRQA | AITSGG | RFTISRDNAK | DSDYSSDYY | WGQ |
| | GRGLVQA | L | PGKQR | STNYAD | NTVYLQMNSL | Y | GTQV |
| | GGSLRLS | | ELVA | SVKG | KPEDTAVYYC | | TVSS |
| | CAASGSIF | | | | AA | | |
| | S | | | | | | |
| 012B03/38 | EVQLVES | YYTI | WFRQA | CISGGD | RFTISRDNAK | DSAGVPAG | WGQ |
| | GGGLVQS | G | PGKERE | TSTYYA | NTVYLQMNSL | PAAVYGSTC | GAQV |
| | GGSLRLS | | GVS | DSVKG | KPEDTAVYYC | SRLEYDY | TVSS |
| | CAASGFA | | | | AT | | |
| | LD | | | | | | |
| 013A02/39 | EVQLVES | SYSM | WFRQA | AINWNG | RFTVSRDNAK | TGWGRAYE | WGQ |
| | GGGLVQA | G | PGKERE | DSTYYE | NTVYLQMNSL | QAYEYDV | GTQV |
| | GGSLRLS | | FIA | DSVKG | KPEDTAVYYC | | TVSS |
| | CAASGGT | | | | AA | | |
| | FS | | | | | | |
| 013A03/40 | EVQLVES | DYAM | WVRQA | TISWND | RFTISRDNAK | GGSRLYDY | WGQ |
| | GGGLVQP | S | PGKGLE | EYTYYA | NTLYLQMNSL | HY | GTQV |
| | GGSLRLS | | WVS | ESMKG | KSEDTAVYYC | | TVSS |
| | CAASGFT | | | | AK | | |
| | VD | | | | | | |
| 014A08/41 | EVQLVES | DYAI | WFRQA | CISSSD | RFTISSDNAK | RPPFHSCSE | WGQ |
| | GGGLVQA | G | PGKERE | GSTYYA | NTVYLQMNSL | YENDY | GTQV |
| | GGSLRLS | | GVS | DSVKG | KPEDTAVYYC | | TVSS |
| | CASSGFT | | | | AA | | |
| | FD | | | | | | |
| 014A09/42 | EVQLVES | SSVL | WFRQA | AISGSG | RFTISRDNAK | GRAFLTRDP | WGQ |
| | GGGLVQA | G | PGKERE | SSTDSA | NTVYLQMNSL | FYYDI | GTQV |
| | GGSLRLS | | FVA | KD | KPEDTAVYYC | | TVSS |
| | CAASGRTI | | | | AA | | |
| | S | | | | | | |
| 014A11/43 | EVQLVES | SVTA | WYRQA | GLSNIG | RFTVSGDAAK | LLWSGNY | WGQ |
| | GGGLVQA | MG | PGKQR | ATKYAD | NTVYLQMNSL | | GTQV |
| | GGSLRLT | | EFVA | SVKG | KPEDTAVYYC | | TVSS |
| | CIASGRDI | | | | NV | | |
| | F | | | | | | |
| 014D03/44 | EVQLVES | DYAI | WFRQA | YISKSD | RFTSSSDNAK | RPTLRVRLD | WGQ |
| | GGGLVQA | G | PGKERE | GTTYYA | NTVYLQINSLK | NDRHHLLYE | GTQV |
| | GGSLRLS | | GVS | DSVKG | PEDTAVYYCA | YEYDY | TVSS |
| | CAASGFT | | | | A | | |
| | FD | | | | | | |
| 014H02/45 | EVQLVES | EYAI | WFRQA | YISSSD | RVTSSSDNAK | RPTLRVRLD | WGQ |
| | GGGLVQA | G | PGKERE | GSTYYA | NTVYLQMNSL | NDRHHLLYE | GTQV |
| | GGSLRLS | | GVS | DSVKG | KPEDTAVYYC | YEYDY | TVSS |
| | CAASGFT | | | | AA | | |
| | FD | | | | | | |
| 015A06/46 | EVQLVES | SYAM | WFRQA | RISWNG | RFTISRDNPK | SIALVGGVT | WGQ |
| | GGGLVQA | G | PGKERE | GSTYHA | NTVYLQMDSL | PHSYDY | GTQV |
| | GGSLRLS | | FVA | DSVKG | KPEDAAIYYC | | TVSS |
| | CAASGRT | | | | AA | | |
| | FS | | | | | | |
| 015C05/47 | EVQLVES | TYLM | WFRQA | VIWSSG | RFTISRDNAK | SYGGNYYIP | WGQ |
| | GGGLVQV | VG | PGKERE | DTDYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |
| 015D05/48 | EVQLVES | TYLM | WFRQA | GIWSSG | RFTISRDNAK | SYDGNYYIP | WGQ |
| | GGGLVQV | VG | PGKERE | GTAYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |
| 015H04/49 | EVQLVES | SYVM | WFRQA | GISWSS | RFTISRDAAE | NSVSEPTLH | WGQ |
| | GGGLVQA | G | PGKELE | GRTYYT | NTWYLQMNS | TWQYEASY | GTQV |
| | GGSLGLS | | FVA | DSVKG | LKPEDTAVYY | DY | TVSS |
| | CAASERT | | | | CAS | | |
| | LP | | | | | | |
| 016A01/50 | EVQLVES | RYAM | WFRRV | HITWNR | RFTISRDKAS | QIKYGEITHP | WGQ |
| | GGGLVQA | G | PGEERE | GSTYYA | NTLYLQMNSL | EEYSY | GTQV |
| | GGSLRLS | | FVT | DSVKG | KPEDTAVYYC | | TVSS |
| | CAASGHT | | | | AA | | |
| | FS | | | | | | |
| 016A02/51 | EVQLVES | RNA | WFRQV | AIRWNV | RFAISRDNAE | YAGLVYSGI | WGQ |
| | GGGLVQA | MG | PGKARE | GRLDYA | NTVYLQMNDL | PDY | GTQV |
| | GGSLRLS | | FVA | DSVKG | KTEDTAVYYC | | TVSS |
| | CAASGRT | | | | AA | | |
| | FS | | | | | | |
| 016A03/52 | EVQLVES | RYAM | WFRRV | HITWNR | RFTISRDKAS | QIKYGEITHP | WGQ |
| | GGGLVQA | G | PGKERE | GSTYYA | NTLYLQMNSL | EEYSY | GTQV |
| | GGSLRLS | | FVT | DSVKG | KPEDTAVYYC | | TVSS |
| | CAASGHT | | | | AA | | |
| | FS | | | | | | |
| 016A05/53 | EVQLVES | RYAM | WFRRV | HITWNR | RFTISRDKAS | QTKYGEITR | WGQ |
| | GGGLVQA | G | PGKERE | GSTYYA | NTLYLQMNSL | PEEYSY | GTQV |
| | GDSLRLS | | FVT | DSVKG | KSEDTAVYYC | | TVSS |
| | CAASGHT | | | | AA | | |
| | FS | | | | | | |
| 016G09/54 | EVQLVES | INAM | WYRQA | FMINDS | RFTISRDSTKN | AYEQHTY | WGQ |
| | GGGLVQA | A | PGKQR | STDYTD | ILYLQMNNLN | | GTQV |
| | GGSLRLV | | EWVA | SVKG | VEDTAVYYCN | | TVSS |
| | CSASGIDF | | | | T | | |
| | S | | | | | | |
| 019A03/55 | EVQLVES | DYAI | WFRQA | CITPSD | RFIISSDNAKN | VPRLRGLGY | WGQ |
| | GGGLVQA | A | PGKERE | DRTYYA | TVYLQMNSLK | WPYPEYEY | GTQV |
| | GGSLRLS | | GIS | DSVKG | PEDTADYYCA | DY | TVSS |
| | CAASGFT | | | | A | | |
| | FD | | | | | | |
| 019G07/56 | KVQLVES | DYAL | WFRQA | CIRCSD | RFTISSDNAK | SIVPRSKLE | WGQ |
| | GGGLVQA | G | AGKERE | GSTYYA | NTVYLQMNSL | PYEYDA | GTQV |
| | GGSLRLS | | GVS | DSVKG | KPEDTAVYYC | | TVSS |
| | CAASGFT | | | | AA | | |
| | FD | | | | | | |
| 019G08/57 | EVQLVES | DYAM | WVRQA | TISWND | RFTISRDNAK | GGSRLYDY | WGQ |
| | GGGLVQP | S | PGKGLE | EYTYYA | NTLYLQMNSL | HY | GTQV |
| | GGSLRLS | | WVS | ESMKG | KSEDTAVYYC | | TVSS |
| | CAVSGFT | | | | AK | | |
| | VD | | | | | | |
| 021G10/58 | EVQLVES | DYAI | WFRQA | CISSSD | RFTVSSDNAK | GLRGRYYR | WGQ |
| | GGGLVQA | G | PGKERE | GSTTYA | NTVYLQMNSL | GTYSLVCAP | GTQV |
| | GGSLRLS | | GVS | DSVKG | KPEDTAVYYC | YEYDF | TVSS |
| | CAASGFT | | | | AA | | |
| | LD | | | | | | |
| 022B09/59 | EVQLVES | DYAI | WFRQA | CISSSD | RFTISADNAK | GLRGRYYS | WGQ |
| | GGGLVQA | G | PGKERE | GSTYYA | NTVYLQMHSL | GSNYLVCAP | GTQV |
| | GGSLRLS | | GVS | DSVKG | KPEDTAVYYC | YEYDY | TVSS |
| | CAASGFT | | | | AA | | |
| | LD | | | | | | |
| 022C07/60 | EVQLVES | DYAI | WFRQA | CISSSD | RFTISSDNAK | DFIISSKRLC | RGQG |
| | GGGLVQA | G | PGKERE | GSTYYA | NTVYLQMNSL | LDLFGS | TQVT |
| | GGSLRLS | | GVS | DSVKG | KPEDTAVYYC | | VSS |
| | CAASGFT | | | | AA | | |
| | FD | | | | | | |
| 022G03/61 | EVQLVES | DYAI | WFRQA | CISSSD | RFTISSDNAK | GITPCSDYT | WGQ |
| | GGGSVQA | G | PGKERE | GSTYYA | NTVYLQMNSL | QTYEYDV | GTQV |
| | GGSLRLS | | GVS | DSVKG | KPGDTAVYYC | | TVSS |
| | CAASGFT | | | | AA | | |
| | FD | | | | | | |
| 022G05/62 | EVQLVES | DYAI | WFRRA | CITSSD | RFTISSDSAKN | VPATRRNP | WGQ |
| | GGGLVQA | G | PGKERE | GSTSYA | TVYLQMNSLK | QMVVAKKC | GTQV |
| | GGSLRLS | | GVS | DSVKG | PEDTAVYYCS | WLAEYEYD | TVSS |
| | CAASGFT | | | | V | Y | |
| | FD | | | | | | |
| 022G11/63 | EVQLVES | DYAI | WFRQA | CISRSD | RFTISSDNAK | SWSGAYYS | WGQ |
| | GGGLVQA | G | PGKERE | GSPYYA | GTVYLQMSSL | GTYYCDRLY | GTQV |
| | GGSLRLS | | GVS | DSVKG | KPEDTAVYYC | EYDA | TVSS |
| | CAVSGFT | | | | AA | | |
| | FD | | | | | | |
| 023A04/64 | EVQLVES | TYLM | WFRQA | TMWSS | RFTISRDNAK | SYGGNYYIP | WGQ |
| | GGGLVQV | VG | PGKERE | GDTAYA | NTVYLQMNSL | GFYED | GTQV |
| | GDSLRLS | | FAA | DSVKG | KAEDTAVYYC | | TVSS |
| | CAASERT | | | | AG | | |
| | FS | | | | | | |
| 023C10/65 | EVQLVES | YYAI | WFRQA | CISSSD | RFTISRDNAK | DSLGYGSS | WGQ |
| | GGGLVQP | G | PGKERE | GSTYYA | NTVYLQMNSL | CRMAPYEY | GTQV |
| | GGSLRLS | | GVS | DSVKG | KPEDTAVYYC | DY | TVSS |
| | CAASGFA | | | | AT | | |
| | LD | | | | | | |
| 023D01/66 | EVQLVES | TYLM | WFRQA | VIWSSG | RFTISRDNAK | SYDGNYYIP | WGQ |
| | GGGLVQV | VG | PGKERE | GTAYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |
| 023E02/67 | EVQLVES | DYAI | WFRQA | CISSSD | RFTISRDNAK | GNRRIYYSD | WGQ |
| | GGGLVQP | G | PGKERE | GITYYA | NTVYLQMNSL | YALACFPYE | GTQV |
| | GGSLRLS | | GVS | DSVKG | KPEDTAVYYC | YDY | TVSS |
| | CAASGFT | | | | AT | | |
| | SD | | | | | | |
| 023E08/68 | EVQLVES | TYLM | WFRQA | GIWSSG | RFTISRDNAK | SYDGNYYIP | WGQ |
| | GRRLVQV | VG | PGKERE | DTAYAD | NTVYLQMNSL | GFYKD | GTQV |
| | GDSLRLA | | FAA | SVKG | KTEDTAVYYC | | TVSS |
| | CAASGRT | | | | AG | | |
| | FS | | | | | | |
| 023F10/69 | EVQLVES | TYLM | WFRQA | TMWVS | RFTISRDNPK | SYGGNYYIP | WGQ |
| | GGGLVQV | VG | PGKERE | GDTAYA | NTVYLQMNSL | GFYKD | GTQV |
| | GDSLRLS | | FAA | DSVKG | KAEDTAVYYC | | TVSS |
| | CAASERT | | | | AG | | |
| | FS | | | | | | |
| 023F11/70 | EVQLVES | TYLM | WFRQA | AIWSSG | RFTISRDNAK | SYGGNYYIP | WGQ |
| | GGGLVQI | VG | PGKERE | DTAVAD | NTVYLQMNSL | GFYED | GTQV |
| | GDSLRLS | | FAA | SVKG | KAEDTAVYYC | | TVSS |
| | CAASGRT | | | | AG | | |
| | FS | | | | | | |
| 024B05/71 | EVQLVES | YYAI | WFRQA | CISSSD | RFTISRDNAK | DSIVCGSYY | WGK |
| | GGGLVQP | G | PGKEW | GSTYYA | NTVYLQMNSL | GMDY | GTQV |
| | GGSLRLS | | EGVS | DSVKG | KPEDTAVYYC | | TVSS |
| | CAASGFT | | | | AT | | |
| | LD | | | | | | |
| 024G05/72 | EVQLVES | TYLM | WFRQA | GIWSSG | RFTVSRDNDK | SYDGNYYIP | WGQ |
| | GGASVQP | VG | PGKERE | GTAYAD | NTVYLQMNSL | GFYKD | GTQV |
| | GGSLRLS | | FAA | SVKE | QAEDTAVYYC | | TVSS |
| | CAASGRT | | | | AG | | |
| | FS | | | | | | |
| 025B01/73 | EVQLVES | DYAI | WFRQA | CISSSD | RFTISSDNAK | AWGASRLPI | WGQ |
| | GGGLVQA | G | PGKERE | GSTYYA | NTVYLQMNSL | GTMPPYEY | GTQV |
| | GGSLRLS | | GVS | DSVKG | KPEDTAVYYC | DY | TVSS |
| | CAASGFT | | | | AA | | |
| | FD | | | | | | |
| 025C06/74 | EVQLVES | INVM | WYRQA | FIGSGG | RFTISRDSTKN | AYEQHTY | WGQ |
| | GGGLVQA | G | PGKQR | STDYID | ILYLRMNNLNV | | GTQV |
| | GGSLRLV | | EWVA | YTDSVK | EDTAVYYCNT | | TVSS |
| | CSASGIDF | | | G | | | |
| | S | | | | | | |
| 025D08/75 | EVQLVES | DYAI | WFRQA | CISSSD | RFTISSDNAK | VNGLGPFSV | WGQ |
| | GGGLVQA | G | PGKDLE | GSTYYA | NTVYLQMNSL | PVPVYDF | GTQV |
| | GGSLRLS | | GVS | DSVKG | KPEDTAVYYC | | TVSS |
| | CAASGFT | | | | AA | | |
| | FD | | | | | | |
| 025F01/76 | EVQLVES | DYVI | WFRQA | CISSSD | RFTISSDNAK | GGPRINIAT | WGQ |
| | GGGLVQA | G | PGKERE | GSTYYA | NTVYLQMNSL | MTCSHDEY | GTQV |
| | GGSLRLS | | GVS | DSVKG | KPEDTAAYYC | EYDY | TVSS |
| | CAVSGLP | | | | AA | | |
| | FD | | | | | | |
| 025F07/77 | EVQLVES | YYAI | WFRQA | CIESSD | RFTISRDNAK | DSAGVPAG | WGQ |
| | GGGLVQP | G | PGKERE | GSTYYA | NTVYLQMNSL | PAAVYGSSC | GTQV |
| | GGSLRLS | | GVS | DSVKG | KPEDTAVYYC | SRLEYDY | TVSS |
| | CAASGFA | | | | AT | | |
| | LD | | | | | | |
| 025G04/78 | EVQLVES | SYDM | WVRQA | AINSRG | RFTISRDNAK | DPYSLSYYG | WGQ |
| | GGGLVQP | S | PGKGPE | GSTYYA | NTLYLQMNSL | YPLYDY | GTQV |
| | GGSLRLS | | WVS | DSVKG | KPEDTAVYYC | | TVSS |
| | CAASGFT | | | | AT | | |
| | FG | | | | | | |
| 025G10/79 | EVQLVES | INVM | WYRQA | FIGSGS | RFSISRDSTK | AYEQHTY | WGQ |
| | GGGLVQA | G | PGKQR | STGYTD | NILYLQMNNL | | GTQV |
| | GGSLRLV | | EWVA | SVKG | NVEDTAVYYC | | TVSS |
| | CSASGIDF | | | | NT | | |
| | S | | | | | | |
| 028D10/80 | EVQLVES | DYAI | WFRQA | CIRDSD | RTISSDNDK | VPAGRLRFG | WGQ |
| | GGGLVQA | G | PGKERE | GSTYYA | NTVYLQMNSL | EQWYPLYE | GTQV |
| | GGSLRLS | | GVS | DSVKG | KPEDTAVYYC | YDA | TVSS |
| | CAASGFT | | | | AA | | |
| | LD | | | | | | |
| 029B08/81 | EVQLVES | SYTM | WFRQT | AISSSLG | RFQILRDNAK | SRSLNLAYT | WGQ |
| | GGGVVQA | A | PGKERD | RTYYAD | ETVWLQMNSL | TKPYDY | GTQV |
| | GDSVRLS | | IVA | SVKG | KPEDTAVYIC | | TVSS |
| | CAASGPT | | | | AA | | |
| | FR | | | | | | |
| 032H10/82 | EVQLVES | DYD | WFRQA | YISSSD | RFTISSDNAK | RPWTRRVY | WGQ |
| | GGGLVQA | MG | PGKERE | GSTYYT | NTVYLQMNSL | GSSWLARS | GTQV |
| | GGSLRLS | | GVS | DSVKG | IPEDTAVYYCA | LDEYEYDY | TVSS |
| | CAASGFD | | | | A | | |
| | FE | | | | | | |
| 036H10/83 | EVQLVES | TYLM | WFRQT | VIWSSG | RFTISRDNAK | SYGGNYYIP | WGQ |
| | GGGLVQV | VG | PGKERE | DTAYAD | NTVYLQMNSL | GFYED | GTQV |
| | GDSLRLS | | FAA | SAKG | KAEDTAVYYC | | TVSS |
| | CAASGHT | | | | AG | | |
| | FS | | | | | | |
| 037A09/84 | EVQLMES | YYAI | WFRQV | CISSSD | RFTISRDNAK | DSGGYIDYD | WGQ |
| | GGGLVQP | G | PGKERE | GITYYV | NTVYLQMNSL | CMGLGYDY | GTQV |
| | GGSLRLS | | GVS | DSVKG | KPGDTAVYYC | | TVSS |
| | CAASGFA | | | | AT | | |
| | LD | | | | | | |
| 037F02/85 | EVQLVES | YYAI | WFRQA | CISSSD | RFTISRDNAK | DSGGYIDYD | WGQ |
| | GGGLVQP | G | PGKERE | GITYYV | NTVYLQMNSL | CMGLGYDY | GTQV |
| | GGSLRLS | | GVS | DSVKG | KPEDTAVYYC | | TVSS |
| | CAASGFA | | | | AT | | |
| | LD | | | | | | |
| 043E10/86 | EVQLVES | TYLM | WFRQA | VIWSSG | RFTISRDNAK | SYDGNYYIP | WGQ |
| | GGGLVQV | VG | PGKERE | DTAYAD | NTVYLQMNSL | GFYKD | GTQV |
| | GDSLRLS | | FAA | SVKG | KAEDTAVYYC | | TVSS |
| | CAASGRT | | | | AG | | |
| | FS | | | | | | |

### Example 5

### Characterization of Purified Anti-Ang2 VHHs

A subset of inhibitory anti-Ang2 VHHs selected from the screening described in Example 4 are further purified and characterized. Selected VHHs are expressed in E. coli TG1 as c-myc, His6-tagged proteins. Expression is induced by addition of 1 mM IPTG and allowed to continue for 4 hours at 37° C. After spinning the cell cultures, periplasmic extracts are prepared by freeze-thawing the pellets. These extracts are used as starting material and VHHs are purified via IMAC and size exclusion chromatography (SEC) resulting in ≥95% purity as assessed via SDS-PAGE.

5.1. Evaluation of hAng2 Blocking VHHs in ELISA

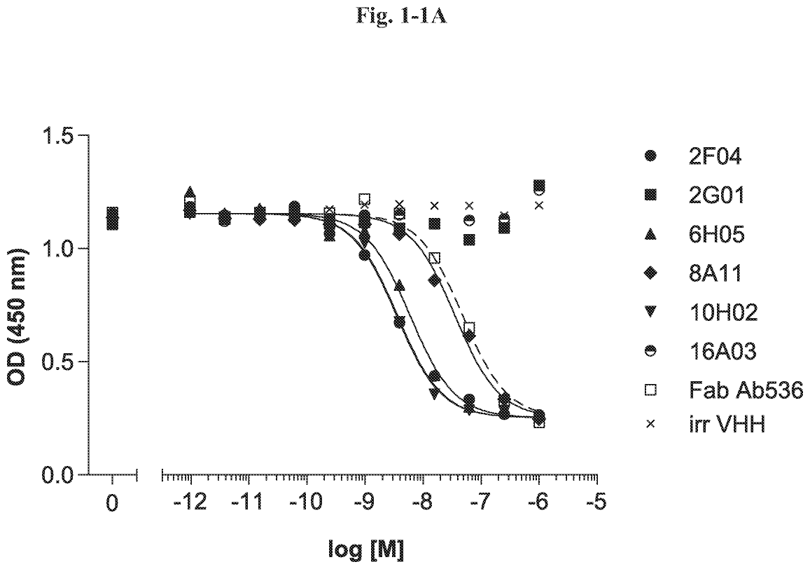

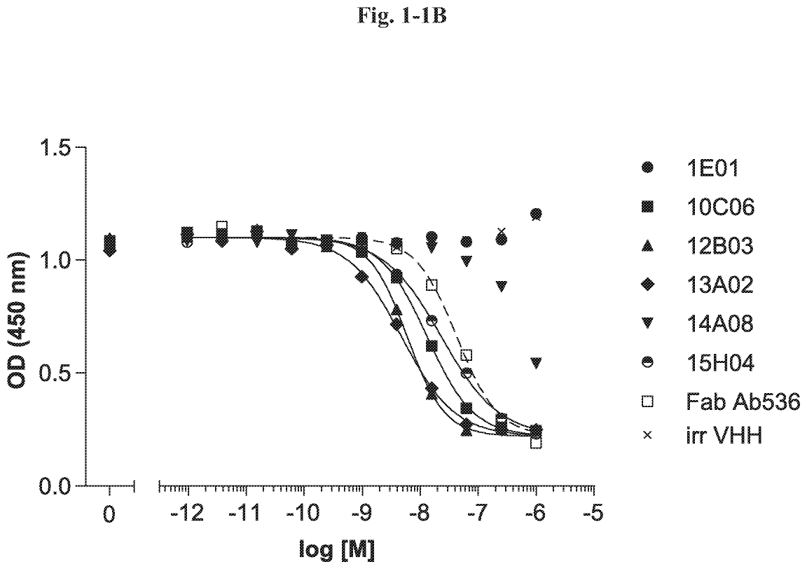

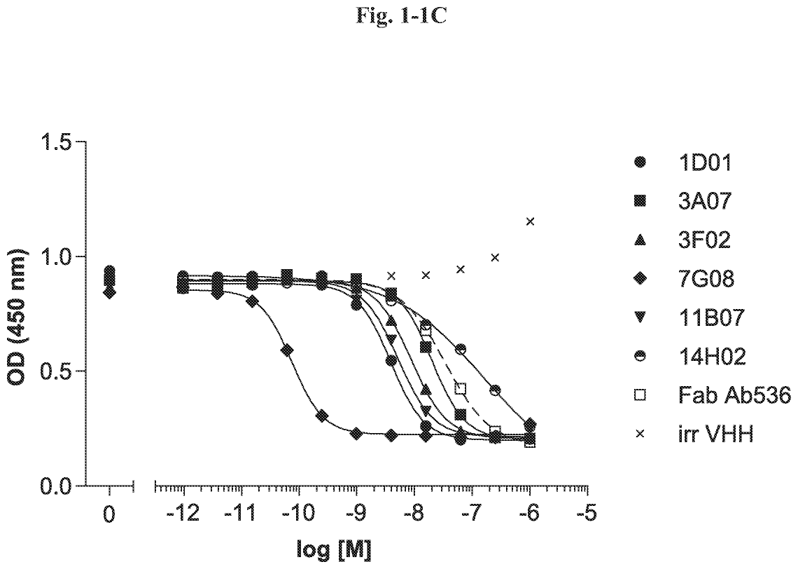

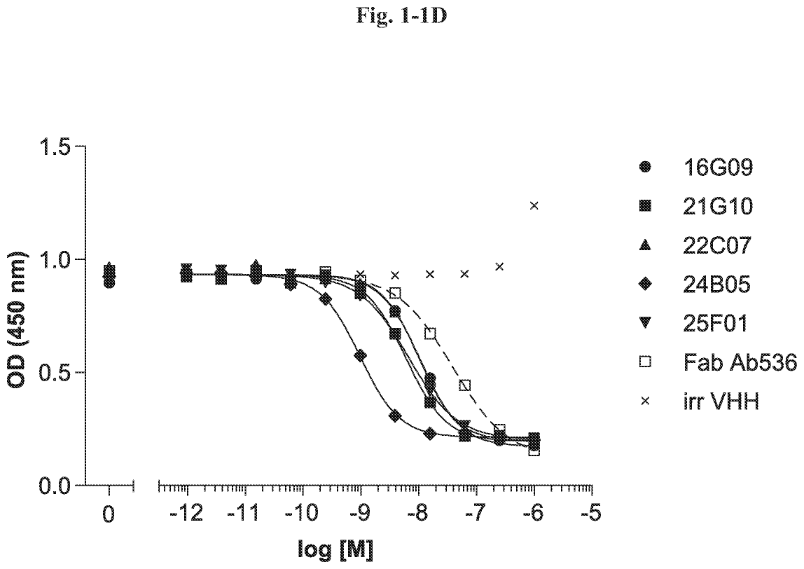

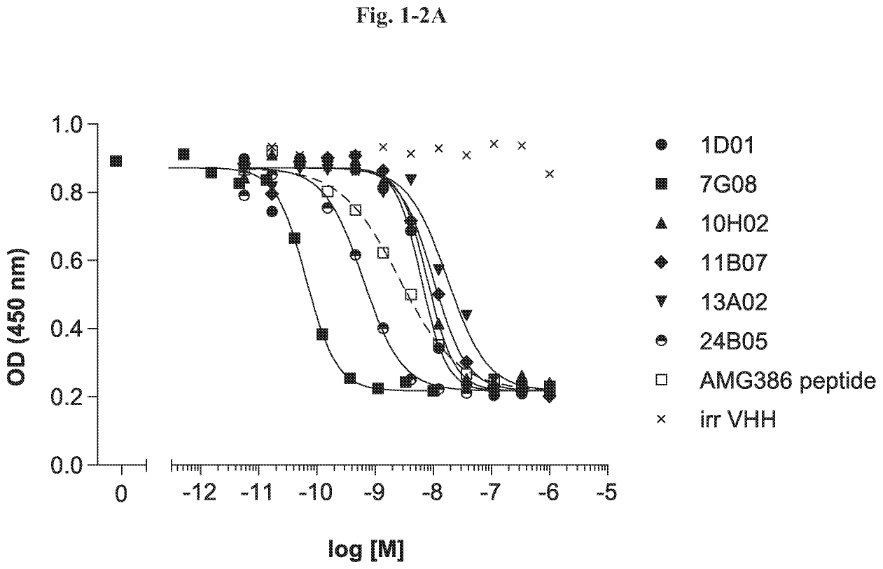

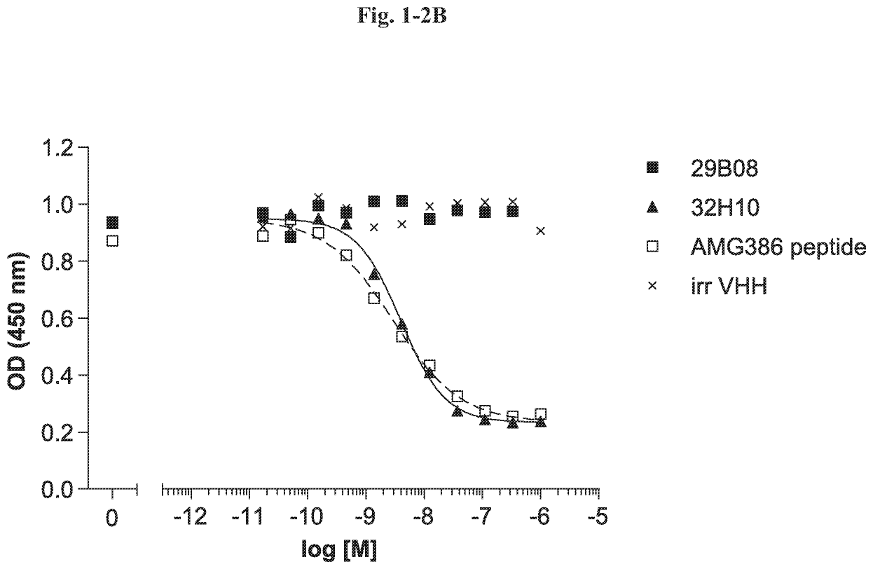

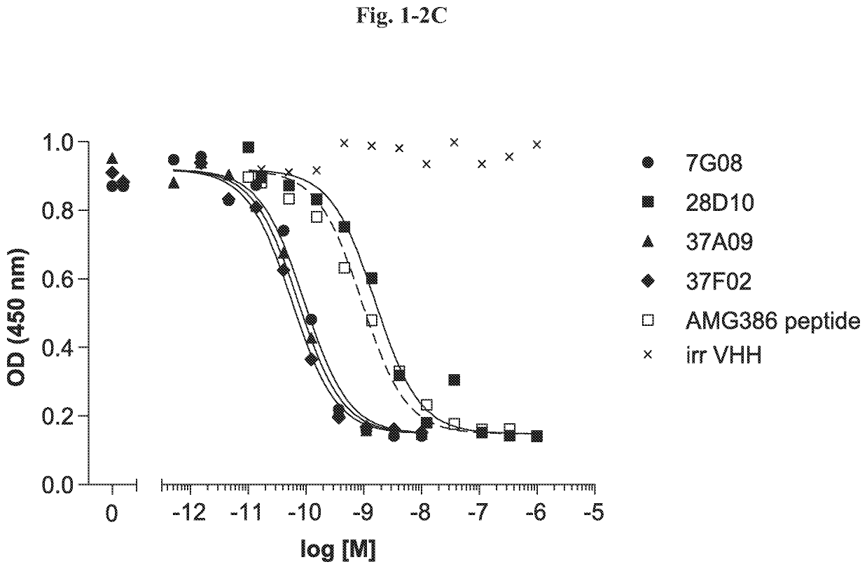

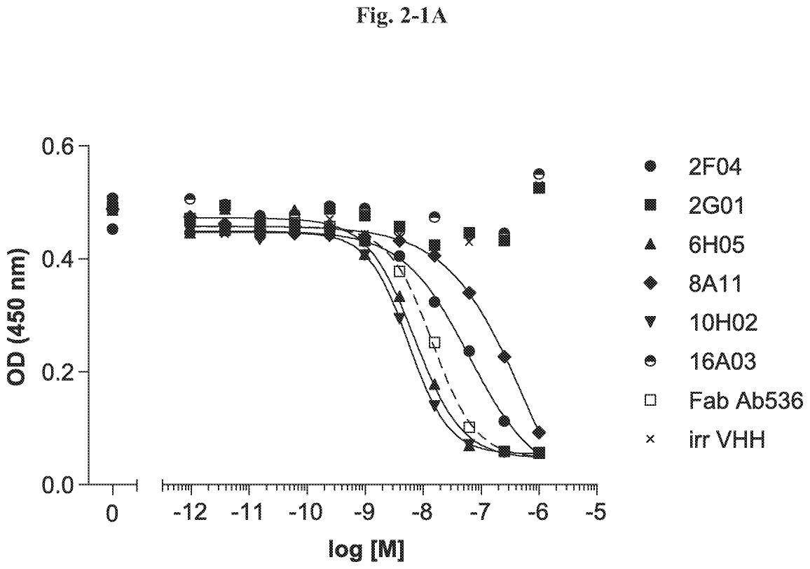

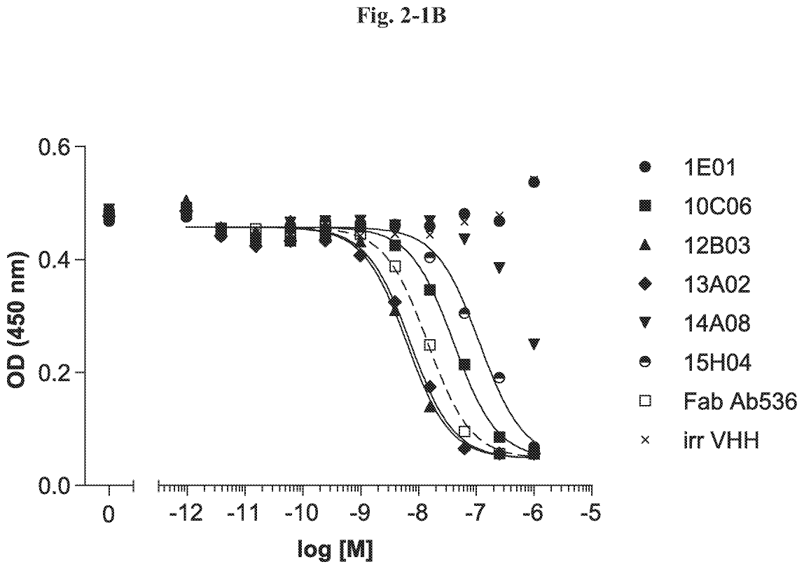

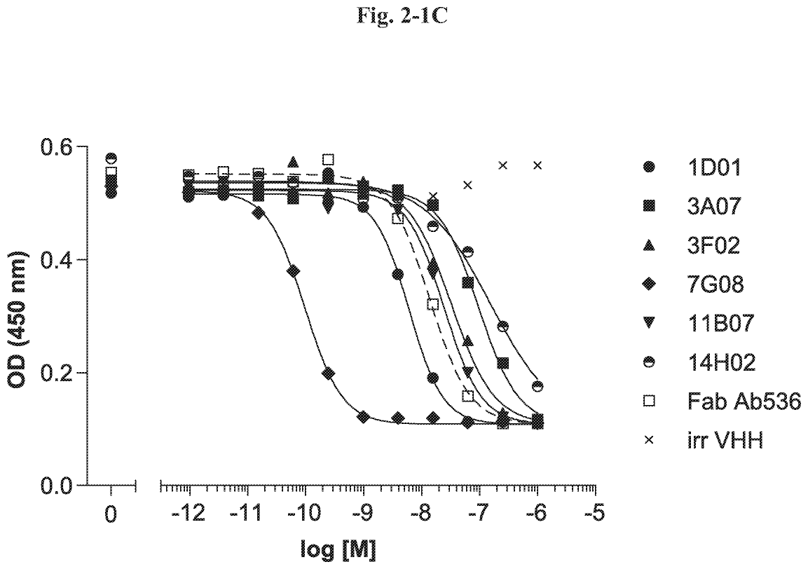

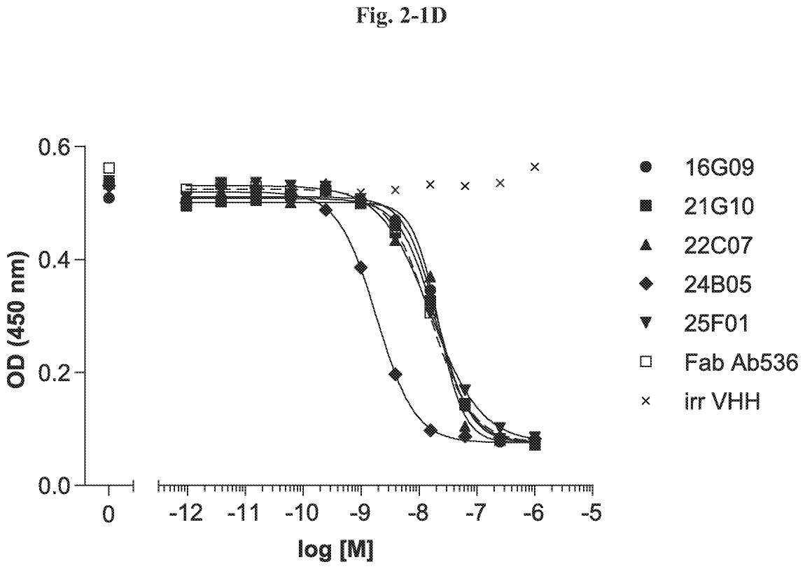

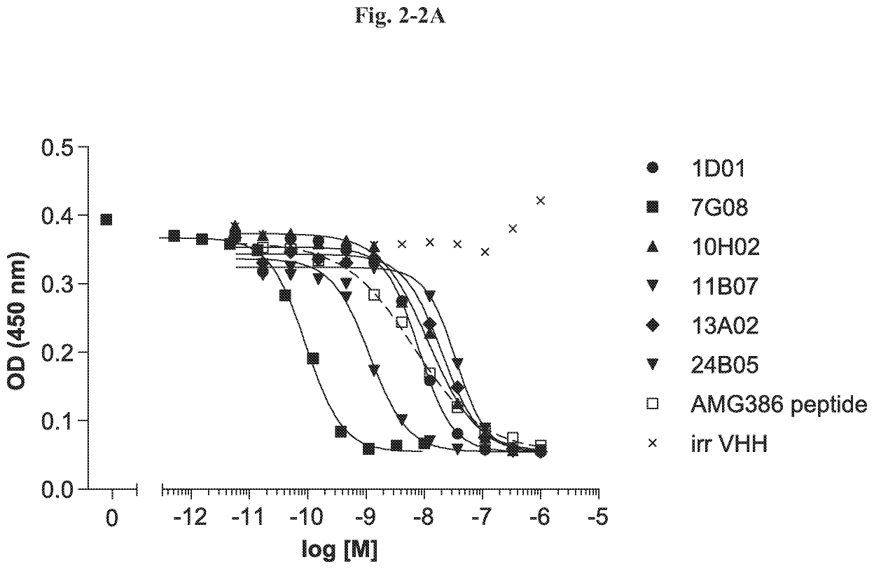

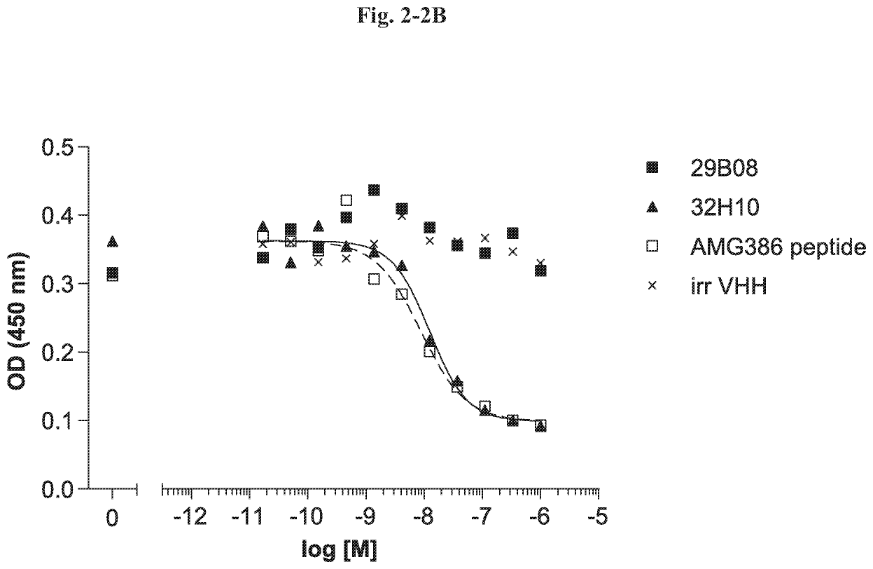

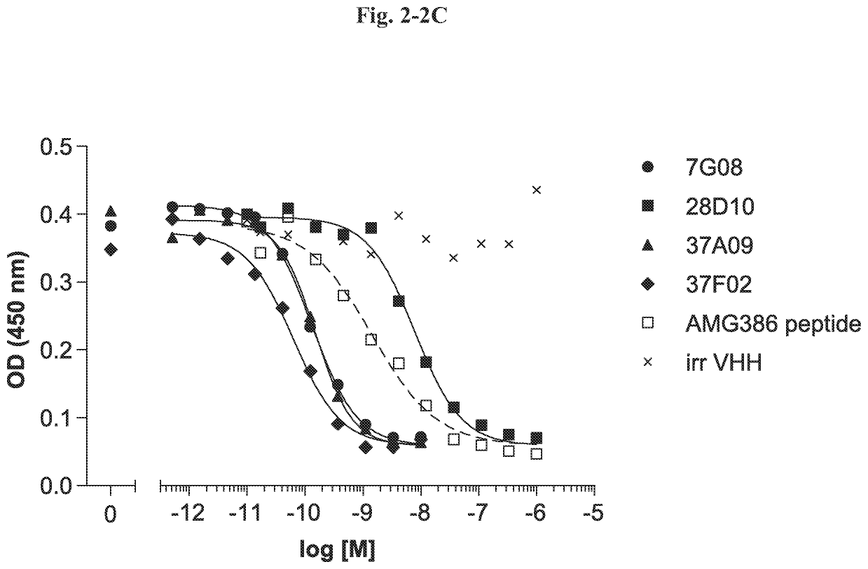

The blocking capacity of the VHHs is evaluated in a human Ang2-human Tie2 blocking ELISA. In brief, 2 μg/mL of Tie2/Fc chimera (R&D Systems, Minneapolis, Minn., USA) is coated in a 96-well MaxiSorp plate (Nunc, Wiesbaden, Germany). A fixed concentration of 0.02 nM FLAG-tagged human Ang2 (Alexis Biochemicals, San Diego, Calif., USA) is added to a dilution series of the purified VHH (diluted in PBS+0.1% casein+0.05% Tween-20), and incubated on the coated human Tie2 receptor for 2 hours. Residual binding of human Ang2 is detected using horseradish peroxidase (HRP) conjugated anti-FLAG M2 (Sigma, St. Louis, Mo., USA) (FIG. 1). Reference molecule is the Fab fragment of Ab536 (US2009/0191212) (FIG. 1-1) or the peptide moiety of peptibody AMG386 (SEQ ID NO:25 in WO2004/092215) (FIG. 1-2). As negative control an irrelevant VHH is used. The IC₅₀ values for VHHs blocking the human Ang2-human Tie2 interaction are depicted in Table 5-1 and Table 5-2, respectively.

TABLE 5-1
IC₅₀ (nM) values of purified VHHs blocking the
hAng2/hTie2 interaction (competition ELISA; VHH:
n = 2 − 3; Fab Ab536: n = 6)

| VHH ID | IC₅₀ (nM) |
|---|---|
| 1D01 | 3.4 |
| 2F04 | 2.8 |
| 3A07 | 21.0 |
| 3F02 | 9.1 |
| 6H05 | 5.3 |
| 7G08 | 0.07 |
| 8A11 | 30.2 |
| 10C06 | 7.7 |
| 10H02 | 3.0 |
| 11B07 | 5.4 |
| 12B03 | 4.6 |
| 13A02 | 4.1 |
| 14H02 | 64.4 |
| 15H04 | 18.6 |
| 16G09 | 11.3 |
| 21G10 | 6.2 |
| 22C07 | 11.0 |
| 24B05 | 1.0 |
| 25F01 | 6.5 |
| Fab Ab536 | 39.3 |

TABLE 5-2
IC₅₀ (nM) values of purified VHHs blocking the hAng2-hTie2
interaction (competition ELISA; VHH: n = 1 − 3;
AMG386 peptide: n = 3)

| VHH ID | IC₅₀ (nM) |
|---|---|
| 1D01 | 6.2 |
| 7G08 | 0.04 |
| 10H02 | 8.7 |
| 11B07 | 14.0 |
| 13A02 | 23.0 |
| 24B05 | 1.1 |
| 28D10 | 1.3 |
| 32H10 | 4.0 |
| 37A09 | 0.1 |
| 37F02 | 0.08 |
| AMG386 peptide | 3.4 |

### 5.2. Evaluation of Cross-Reactivity Towards Mouse and Cynomolgus Ang2 in Blocking ELISA

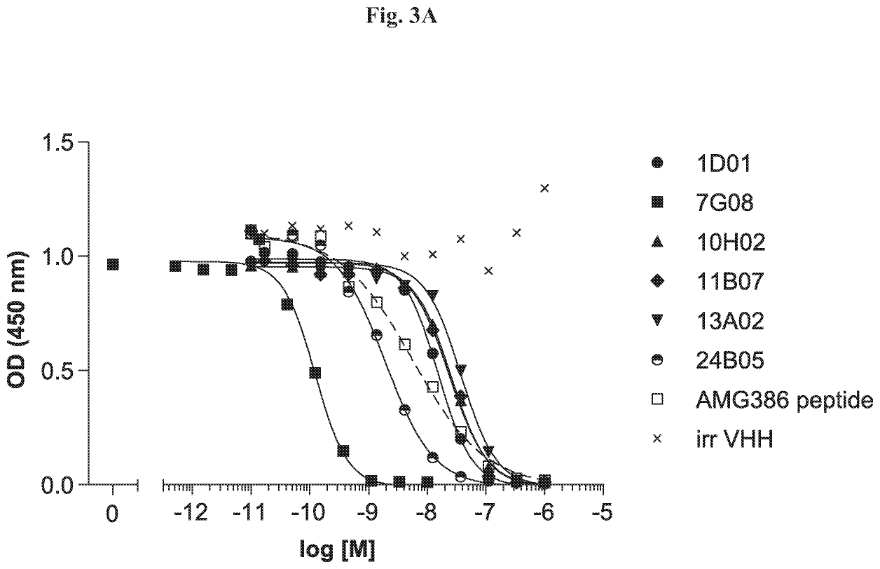

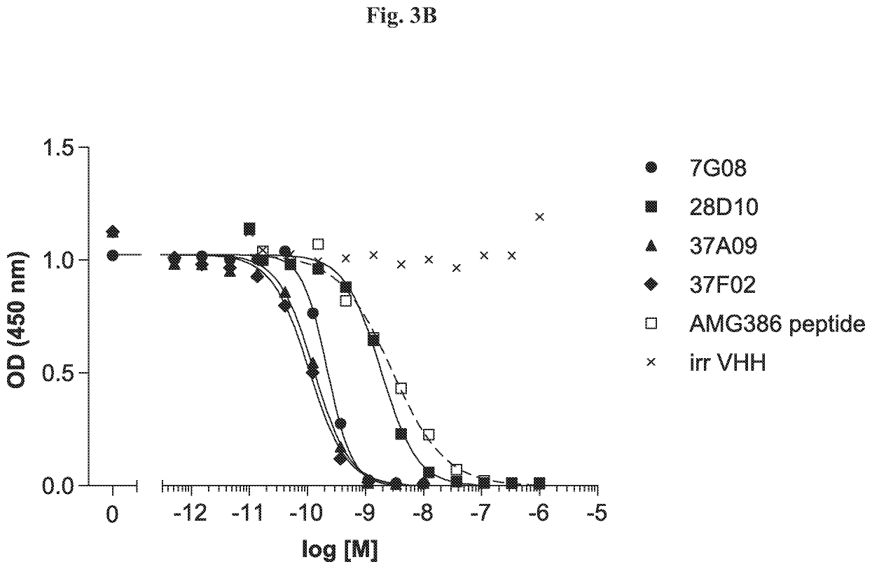

In order to determine if the VHH inhibits binding of mouse Ang2 to mouse Tie2 and cyno Ang2 to cyno Tie2, a competition ELISA is performed. In brief, 2 μg/mL of recombinant mouse Tie2-Fc or cyno Tie2-Fc is coated overnight at 4° C. in a 96-well MaxiSorp plate (Nunc, Wiesbaden, Germany). Coated wells are blocked with a 1% casein solution. FLAG-tagged mouse Ang2 (1:3,000 dilution of conditioned medium from transient HEK transfection) or FLAG-tagged cyno Ang2 (1:800 dilution of conditioned medium from transient HEK transfection) and a dilution series of purified VHH (diluted in PBS+0.1% casein+0.05% Tween-20) are incubated on the coated Tie2-Fc receptor for 2 hours at room temperature to reach binding equilibrium. Residual binding of FLAG-mAng2 or FLAG-cAng2 is detected using HRP conjugated anti-FLAG M2 mAb (Sigma, St. Louis, Mo., USA). Reference molecule is the Fab fragment of Ab536 (mouse: FIG. 2-1) or the peptide moiety of peptibody AMG386 (mouse: FIG. 2-2; cyno: FIG. 3). As negative control an irrelevant VHH is used. The IC₅₀ values for VHHs blocking the mouse Ang2-mouse Tie2 interaction are depicted in Table 6-1. The IC₅₀ values for VHHs blocking the mouse and cyno Ang2 binding to mouse and cyno Tie2, respectively, is shown in Table 6-2.

TABLE 6-1
IC₅₀ (nM) values of purified VHHs blocking the interaction
of mAng2 to mTie2 (competition ELISA; VHH: n = 2 −
3; Fab Ab536: n = 5)

| | mAng2 |
|---|---|
| VHH ID | IC₅₀ (nM) |
| 1D01 | 6.3 |
| 2F04 | 57.4 |
| 3A07 | 99.3 |
| 3F02 | 32.7 |
| 6H05 | 7.7 |
| 7G08 | 0.09 |
| 8A11 | 442.1 |
| 10C06 | 45.2 |
| 10H02 | 5.2 |
| 11B07 | 21.0 |
| 12B03 | 6.7 |
| 13A02 | 6.1 |
| 14H02 | 143.2 |
| 15H04 | 124.6 |
| 16G09 | 19.4 |
| 21G10 | 16.8 |
| 22C07 | 13.6 |
| 24B05 | 1.5 |
| 25F01 | 13.3 |
| Fab Ab536 | 15.3 |

TABLE 6-2
IC₅₀ (nM) values of purified VHHs blocking the interaction of mAng2
and cAng2 to mTie2 and cTie2, respectively (competition ELISA;
VHH: n = 1 − 3; AMG386 peptide: n = 3; n.d., not determined)

| | mAng2 | cAng2 |
|---|---|---|
| VHH ID | IC₅₀ (nM) | IC₅₀ (nM) |
| 1D01 | 10.0 | 16.4 |
| 7G08 | 0.07 | 0.14 |
| 10H02 | 21.4 | 23.7 |
| 11B07 | 39.7 | 24.8 |
| 13A02 | 26.6 | 33.1 |
| 24B05 | 1.1 | 2.1 |
| 28D10 | 6.1 | 2.0 |
| 32H10 | 13.0 | n.d. |
| 37A09 | 0.1 | 0.2 |
| 37F02 | 0.09 | 0.1 |
| AMG386 peptide | 5.2 | 6.6 |

### 5.3. Evaluation of Selectivity of Human Ang2 Blocking VHHs Towards Human Ang1 in ELISA

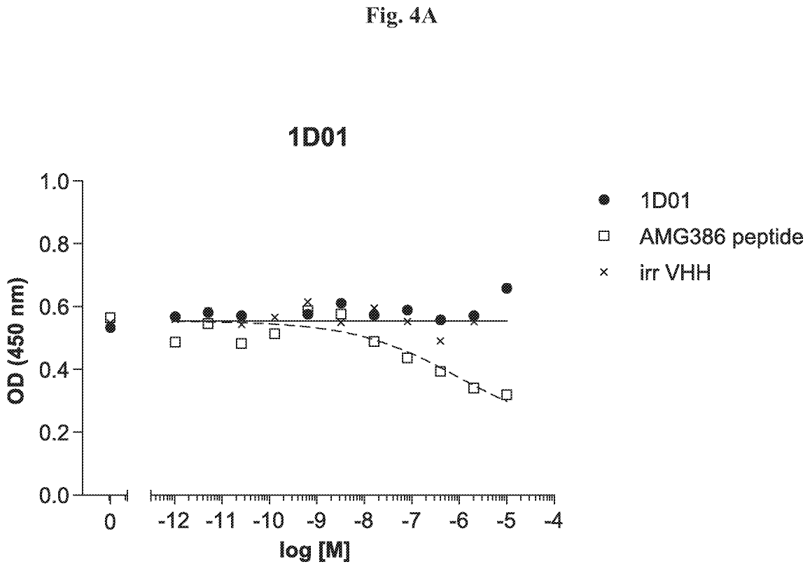

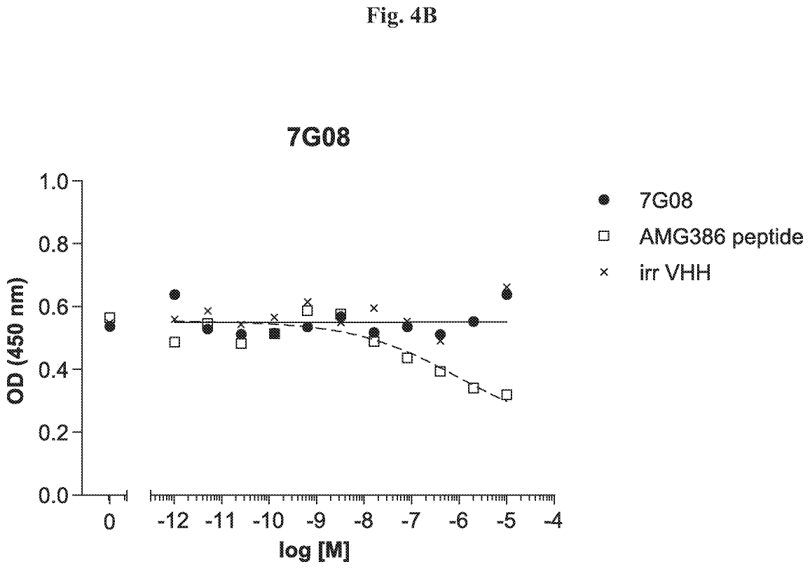

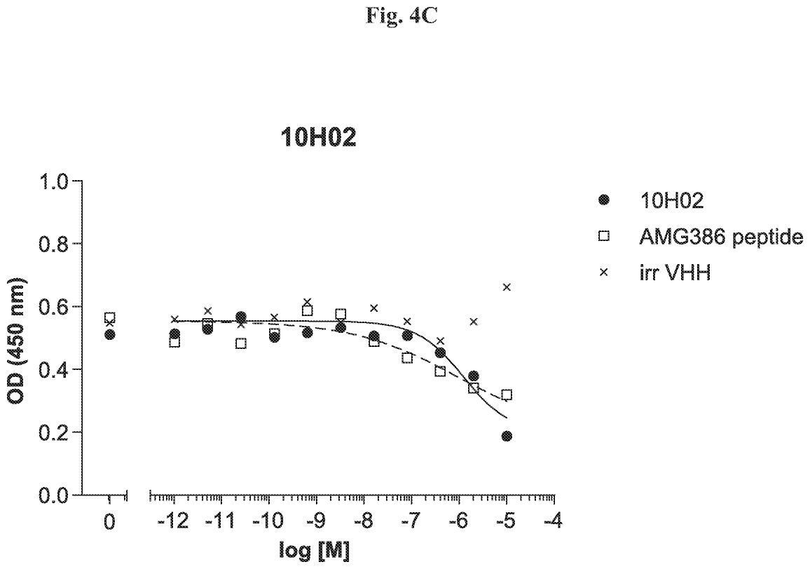

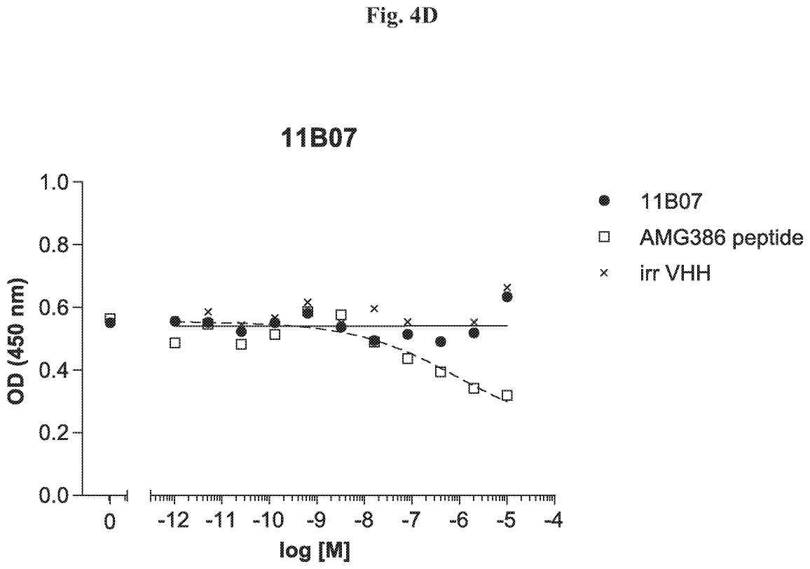

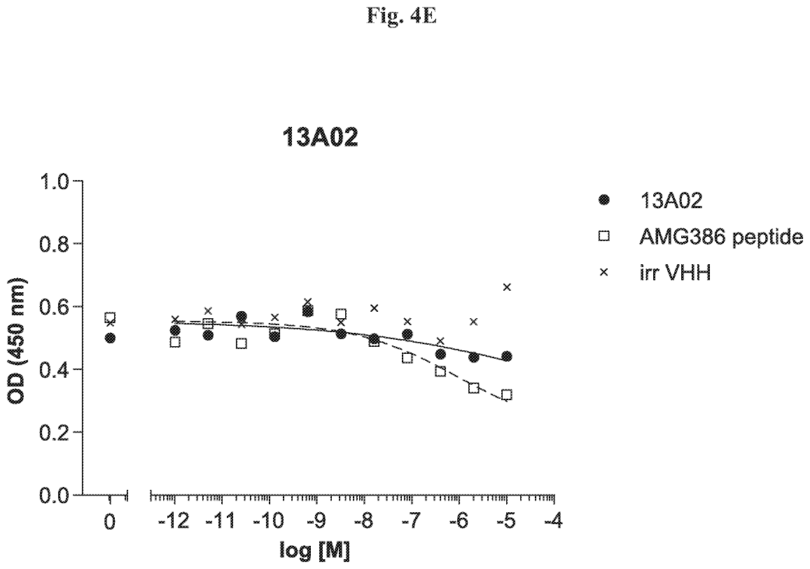

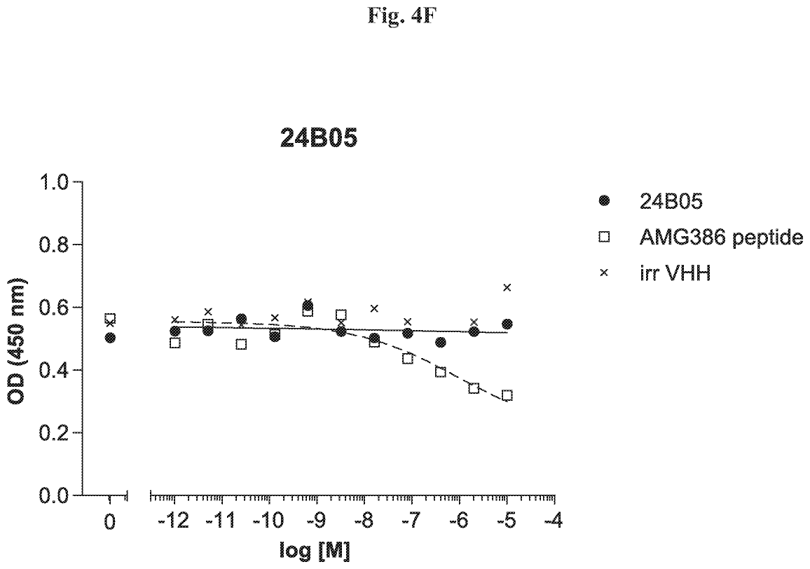

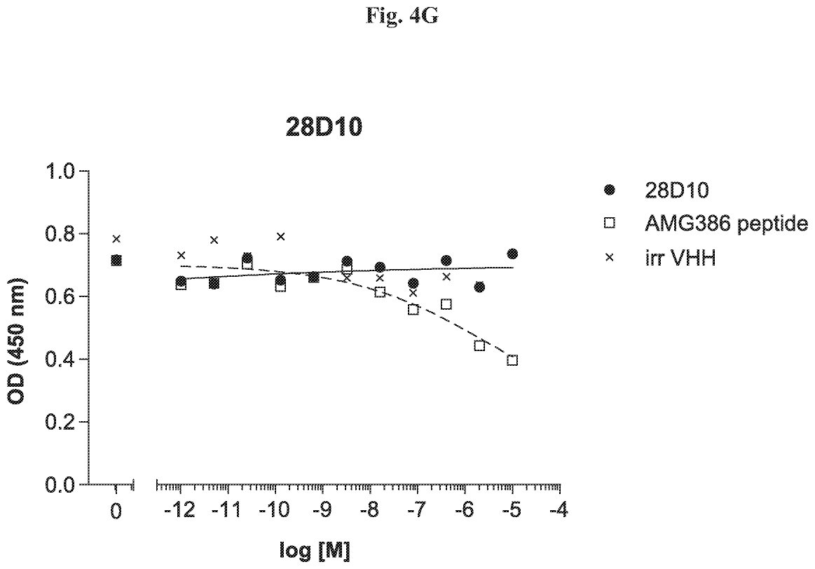

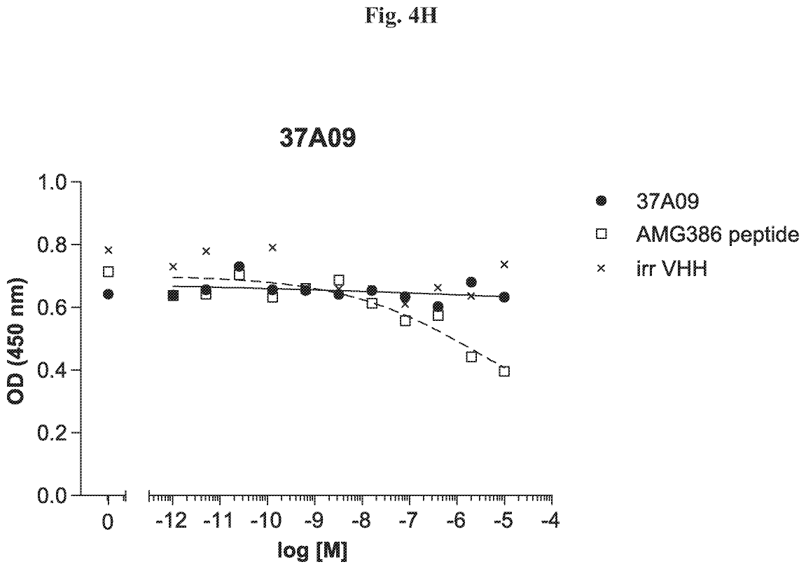

In order to determine whether the anti-Ang2 blocking VHHs are selective over human Ang1 binding to human Tie2, a competition ELISA is performed. In brief, 2 μg/mL of recombinant human Tie2-Fc (R&D Systems, Minneapolis, Minn., USA) is coated overnight at 4° C. in a 96-well MaxiSorp plate (Nunc, Wiesbaden, Germany). Coated wells are blocked with a 1% casein solution. A fixed concentration (0.02 nM) of FLAG-tagged recombinant human Ang1 (Alexis Biochemicals, San Diego, Calif., USA) and a dilution series of VHH (diluted in PBS+0.1% casein+0.05% Tween-20) are incubated on the coated receptor Tie2-Fc for 2 hours at room temperature to reach binding equilibrium. Residual binding of FLAG-hAng1 is detected using HRP conjugated anti-FLAG M2 mAb. Reference molecule is the peptide moiety of AMG386 peptibody (FIG. 4). As negative control an irrelevant VHH is used. The indicative IC₅₀ values for VHHs blocking the human Ang1-human Tie2 interaction are depicted in Table 7.

TABLE 7
IC₅₀ (nM) values of purified VHHs blocking the interaction
of human Ang1 to human Tie2 (competition ELISA;
VHH: n = 2 − 3; AMG386 peptide: n = 3)

| | | hAng1/hAng2 |
|---|---|---|
| VHH ID | IC₅₀ (nM) | ratio |
| 1D01 | >67,000 | >10,800 |
| 7G08 | >84,000 | >2,333,333 |
| 10H02 | 2,000 | 230 |
| 11B07 | 120,000 | 8,570 |
| 13A02 | 17,000 | 739 |
| 24B05 | 120,000 | 109,090 |
| 28D10 | >4,000 | >3,076 |
| 37A09 | >10,000 | >100,000 |
| 37F02 | >10,000 | >100,000 |
| AMG386 peptide | 4,000 | 1,176 |

### 5.4. Determining the Affinity of the Human, Mouse, Cyno Ang2—VHH Interaction

Affinities of the VHH for binding to human, mouse and cyno Ang2 are determined using surface plasmon resonance (SPR) analysis (Biacore T100). In brief, VHH and benchmark compounds are immobilized on a CM5 chip via amine coupling. A multi-cycle kinetic approach is used: different concentrations of human, mouse and cyno Ang2-FLD (0.4-1-2.6-6.4-16-40-100 nM) are injected. Ang2-FLD species are allowed to associate for 2 min and to dissociate for 20 min at a flow rate of 45 μL/min. In between injections, the surfaces are regenerated with a 10 sec pulse of 25 mM NaOH and 60 sec stabilization period. Association/dissociation data are evaluated by fitting a 1:1 interaction model (Langmuir binding). The affinity constant K_{D} is calculated from resulting association and dissociation rate constants kₐ and k_{d} and are depicted in Table 8.

TABLE 8
Affinity KD (nM) of purified VHHs for human, mouse and cyno Ang2

| | human Ang2-FLD | | | mouse Ang2-FLD | | | cyno Ang2-FLD | | |
|---|---|---|---|---|---|---|---|---|---|
| VHH | kₐ | k_{d} | K_{D} | kₐ | k_{d} | K_{D} | kₐ | k_{d} | K_{D} |
| ID | (1/Ms) | (1/s) | (M) | (1/Ms) | (1/s) | (M) | (1/Ms) | (1/s) | (M) |
| 1D01 | 7.7E+06 | 1.5E−02 | 2.0E−09 | 3.3E+06 | 1.5E−02 | 4.6E−09 | 9.9E+06 | 1.4E−02 | 1.4E−09 |
| 7G08 | 1.0E+06 | 1.0E−04 | 9.7E−11 | 8.1E+05 | 1.4E−04 | 1.8E−10 | 1.5E+06 | 1.1E−04 | 7.2E−11 |
| 10H02 | 5.7E+06 | 2.1E−02 | 3.6E−09 | 2.5E+06 | 2.6E−02 | 1.1E−08 | 6.9E+06 | 2.3E−02 | 3.4E−09 |
| 11B07 | 9.2E+06 | 6.2E−02 | 6.7E−09 | 4.8E+06 | 1.4E−01 | 2.9E−08 | 1.2E+07 | 7.3E−02 | 6.1E−09 |
| 13A02 | 9.2E+06 | 9.1E−02 | 9.9E−09 | 1.9E+06 | 3.4E−02 | 1.8E−08 | 1.1E+07 | 9.4E−02 | 8.7E−09 |
| 24B05 | 2.6E+06 | 2.5E−03 | 9.6E−10 | 1.7E+06 | 2.9E−03 | 1.7E−09 | 4.1E+06 | 2.4E−03 | 5.9E−10 |
| 28D10 | 4.9E+06 | 6.2E−03 | 1.3E−09 | 1.9E+06 | 1.1E−02 | 5.6E−09 | 1.8E+07 | 2.5E−02 | 1.4E−09 |
| mAb | 5.0E+07 | 5.5E−02 | 1.1E−09 | 1.1E+07 | 6.1E−02 | 5.5E−09 | n.d. | n.d. | n.d. |
| 3.19.3 | | | | | | | | | |
| Fab | 3.1E+06 | 3.7E−02 | 1.2E−08 | 1.6E+06 | 1.7E−02 | 1.1E−08 | 4.1E+06 | 4.5E−02 | 1.1E−08 |
| Ab536 | | | | | | | | | |

### Example 6

### Affinity Maturation of Selected VHH

A variant of VHH 28D10 (00027 carrying C₅₀S/S₅₃N and Q₁₀₈L substitution—Example 7.3) is subjected to affinity maturation.

In a first cycle, amino acid substitutions are introduced randomly in both framework (FW) and complementary determining regions (CDR) using the error-prone PCR method. Mutagenesis is performed in a two-round PCR-based approach using the Genemorph II Random Mutagenesis kit (Stratagene, La Jolla, Calif., USA) using 1 ng of VHH 00027 cDNA template, followed by a second error-prone PCR using 0.1 ng of product of round 1. After a polish step, PCR products are inserted via unique restriction sites into a vector designed to facilitate phage display of the VHH library. Consecutive rounds of in-solution selections are performed using decreasing concentrations of biotinylated recombinant human Ang2 (R&D Systems, Minneapolis, Minn., USA) and trypsin elutions. Periplasmic extracts (in a volume of ˜80 uL) are prepared according to standard methods and screened for binding to recombinant human Ang2-FLD in a ProteOn (BioRad, Hercules, Calif., USA) off-rate assay. In brief, a GLC ProteOn Sensor chip is coated with recombinant human Ang2-FLD on the “ligand channels” L3, L4, L5 and L6 (with L1/L2 as reference channel). Periplasmic extract of affinity matured clones is diluted 1:10 and injected across the “analyte channels” A1-A6. An average off-rate is calculated of the reference VHH 00027 which is prepared and tested in the same way as the affinity matured VHHs and serves as a reference to calculate off-rate improvements. The top 25 affinity matured variants are shown in Table 9. VHH are sequenced (Table 10-A) to identify amino acid mutations beneficial for improving the off-rate (Table 10-B).

TABLE 9
Off-rate and fold improvement of affinity
matured variants of VHH 00027.

| VHH ID | k_{d} (1/s) | fold improvement |
|---|---|---|
| 64G03 | 7.4E−05 | 15.7 |
| 64F03 | 1.1E−04 | 10.8 |
| 64D11 | 1.1E−04 | 10.3 |
| 64G11 | 1.2E−04 | 9.6 |
| 55D06 | 1.2E−04 | 9.4 |
| 64F07 | 1.2E−04 | 9.4 |
| 64G02 | 1.2E−04 | 9.4 |
| 55A06 | 1.3E−04 | 9.2 |
| 64C03 | 1.3E−04 | 9.2 |
| 64G12 | 1.3E−04 | 8.9 |
| 65F03 | 1.4E−04 | 8.9 |
| 55F02 | 1.4E−04 | 8.7 |
| 64E12 | 1.3E−04 | 8.7 |
| 60C09 | 1.6E−04 | 8.5 |
| 64B02 | 1.4E−04 | 8.5 |
| 64A03 | 1.4E−04 | 8.3 |
| 64C07 | 1.4E−04 | 8.3 |
| 60A06 | 1.6E−04 | 8.3 |
| 64B01 | 1.4E−04 | 8.2 |
| 64G01 | 1.4E−04 | 8.2 |
| 56A07 | 1.5E−04 | 8.1 |
| 58D10 | 1.3E−04 | 8.1 |
| 65F01 | 1.6E−04 | 8.1 |
| 53A06 | 1.6E−04 | 8.0 |
| 55G03 | 1.5E−04 | 8.0 |

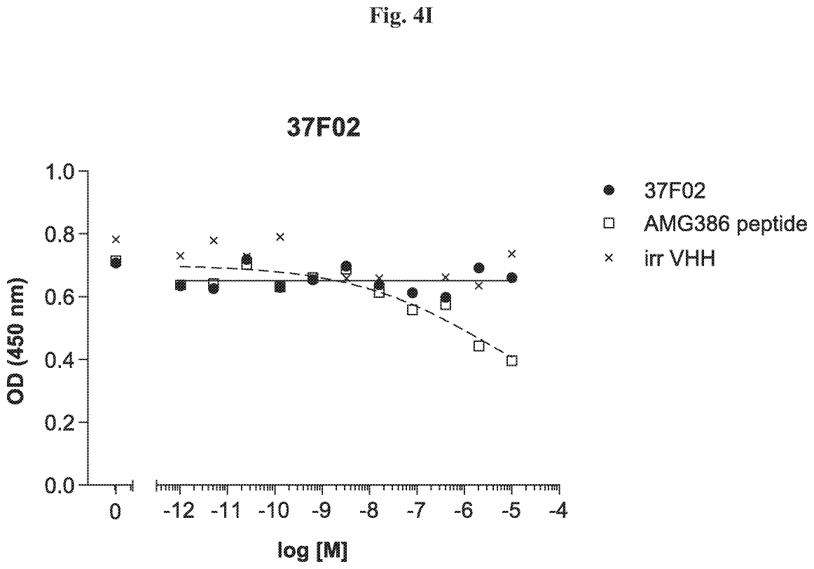

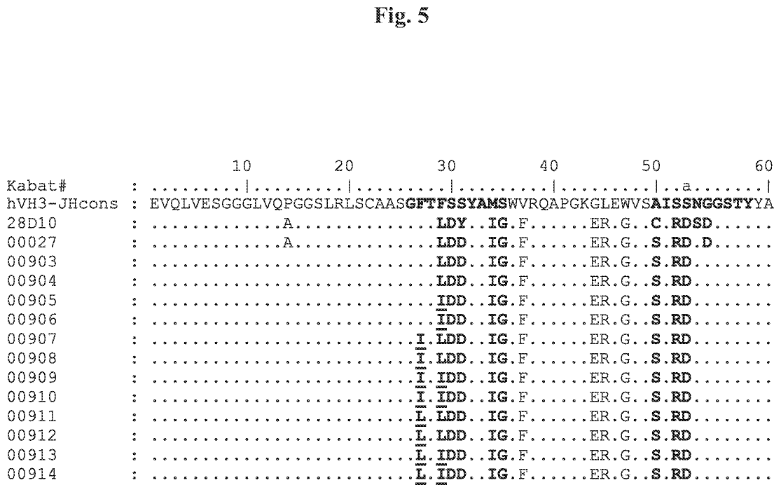

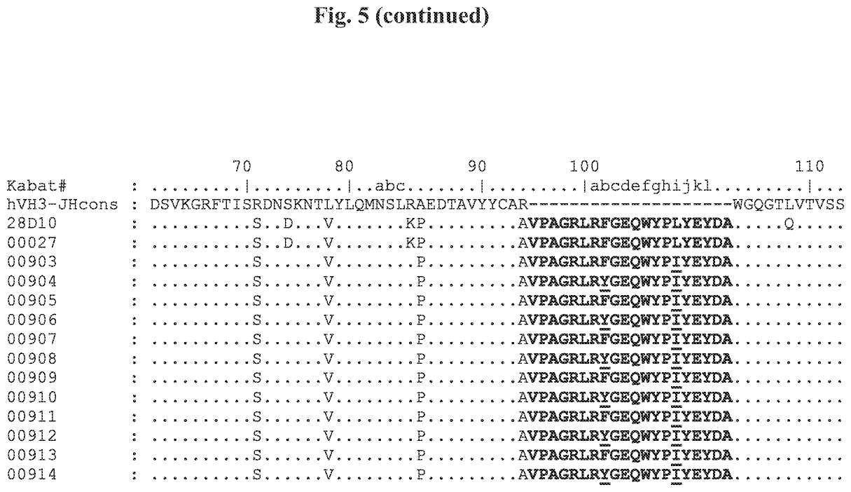

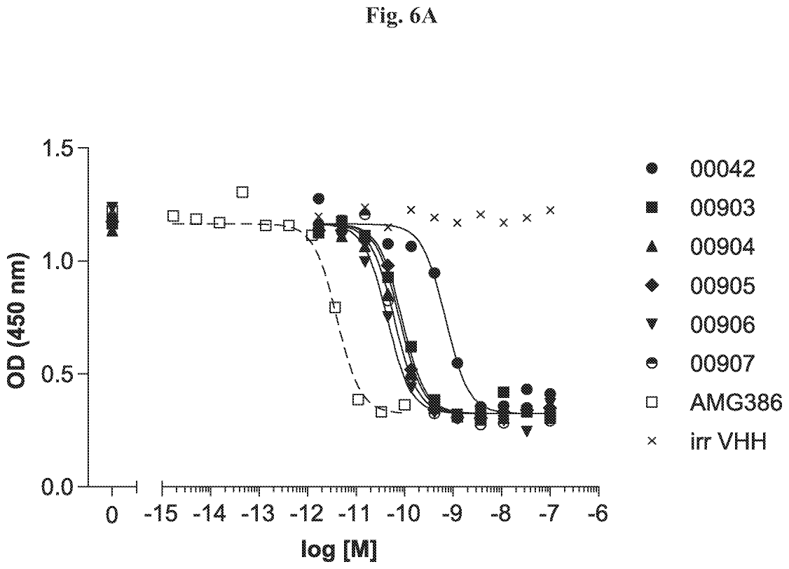

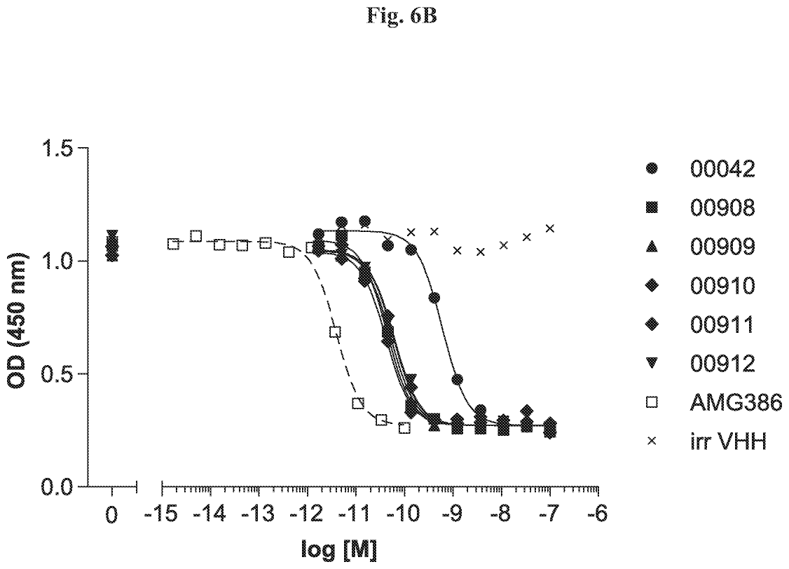

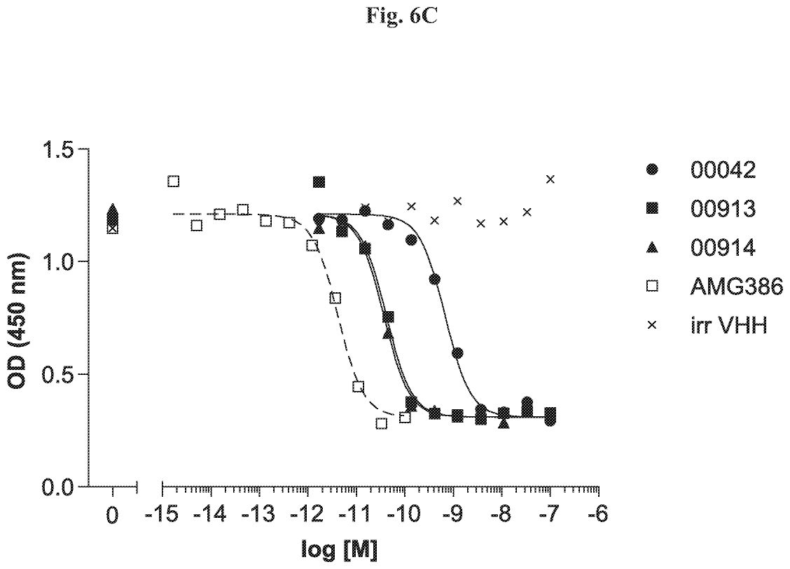

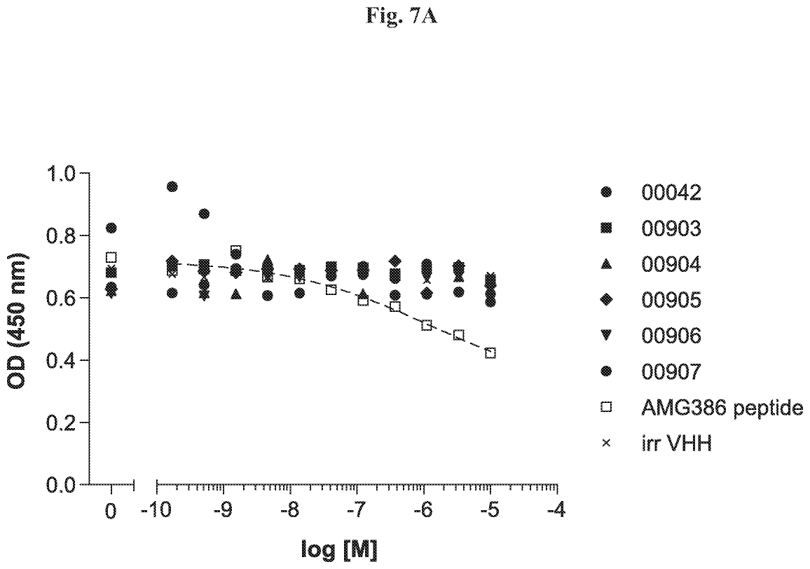

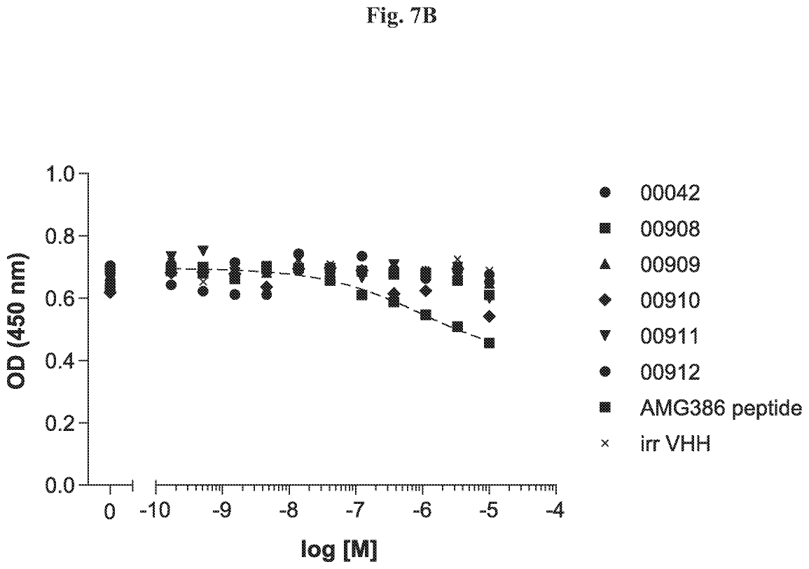

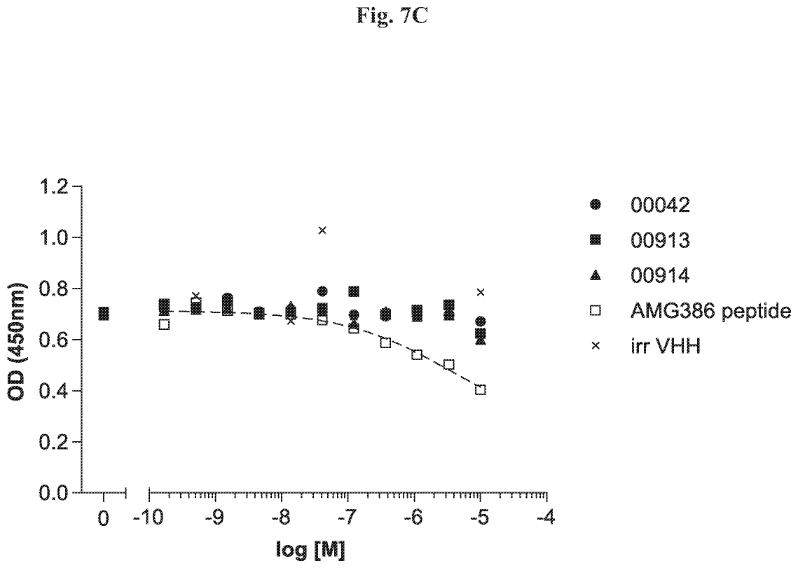

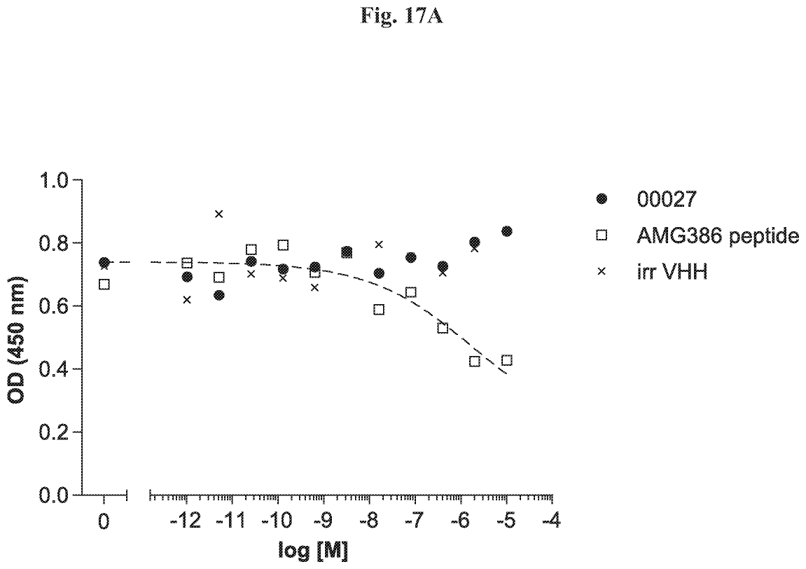

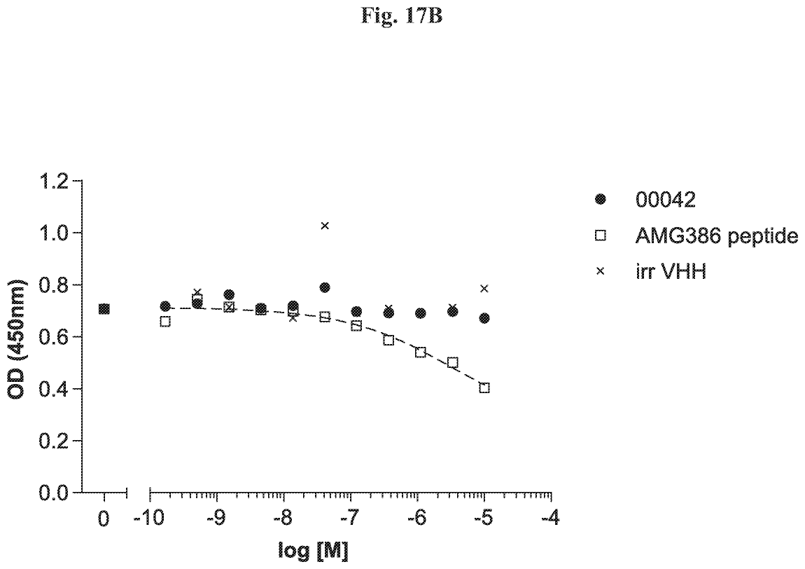

Initially 12 VHH variants containing combinations of mutations on Kabat position 27, 29, 100b and 100i (Table 11; FIG. 5) are constructed. The different combinations of these 4 mutations are grafted on the sequence optimized VHH 00042 backbone (FIG. 17-B) containing an additional D₅₄G substitution (Example 6.3). The amino acid sequence is aligned to the human germline VH3/JH consensus sequence. Residues are numbered according to Kabat, CDRs are shown in grey according to AbM definition (Oxford Molecular's AbM antibody modeling software). Constructs are cloned into the expression vector pAX100 in frame with a C-terminal c-myc tag and a (His)6 tag. VHH variants are produced in E. coli and purified by IMAC and SEC. Sequences are represented in Table 11. All these VHH are analysed in the hAng2/hTie2 (Example 5.1; results shown in FIG. 6 and Table 12), and hAng1/hTie2 competition ELISA (Example 5.3; results shown in FIG. 7 and Table 12). Additionally, the melting temperature (Tₘ) of each variant at pH7 is determined in a thermal shift assay, which is based on the temperature dependent change in fluorescence signal upon incorporation of Sypro Orange (Invitrogen, Carlsbad, Calif., USA) (Ericsson et al, Anal. Biochem. 357 (2006), pp 289-298) (Table 12).

TABLE 10-A
Amino acid sequence of affinity matured anti-Ang2 VHHs

| VHH ID/ | | | | | | | |
|---|---|---|---|---|---|---|---|
| SEQ ID | | | | | | | |
| NOs: | FR1 | CDR1 | FR2 | CDR2 | FR3 | CDR3 | FR4 |
| 64G03/87 | EVQLVES | DYAI | WFRQA | SIRDND | RSTISSDNDK | VPAGRLRY | WGQ |
| | GGGLVQA | G | PGKERE | GSTYYA | NTVYLQMDSL | GEQWYPIYE | GTLV |
| | GGSLRLS | | GVS | DSVKG | KPEDTAVYYC | YDA | TVSS |
| | CAASGFT | | | | AA | | |
| | LD | | | | | | |
| 64F03/88 | EVQLVES | DYAV | WFRQA | TIRDND | RFTISSDNDK | VPAGRLRFG | WGQ |
| | GGGLVQA | G | PGKERE | GSTYYA | NTVYLQMNSL | EQWYPIYEY | GTLV |
| | GGSLRLS | | GVS | DSVKG | KPKDTAVYYC | DA | TVSS |
| | CAVSGITL | | | | AA | | |
| | D | | | | | | |
| 64D11/89 | EVQLVES | DYAI | WFRQA | SIRDND | RFTISSDNDK | VPAGRLRFG | WGQ |
| | GGGQAQ | G | PGKERE | GSTYYT | NTVYLQMNSL | EQWYPIYEY | GTLV |
| | AGGSLRL | | GVS | DSVKG | KPEDTAVYYC | DA | TVSS |
| | SCAVSGF | | | | AA | | |
| | TLD | | | | | | |
| 64G11/90 | EVQLVES | DYAI | WFRQA | SIRDND | RFTISSDNDK | VPAGRLRFG | WGQ |
| | GGGLVQA | G | PGKERV | GSTYYA | NTVYLQMNSL | EQWYPIYEY | GTLV |
| | GGSLRLS | | GVS | DSVKG | KPEDTAVYYC | DA | TVSS |
| | CAASGITL | | | | AA | | |
| | D | | | | | | |
| 55D06/91 | EVQLVES | DYAI | WFRQA | SIRDND | RFTISSDNDK | VPAGRLRFG | WGQ |
| | GGGLVQA | G | PGKERE | GSTYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 64F07/92 | QVQLVES | DYAI | WFRQA | SIRDND | RFTISSDNDQ | VPAGRLRFG | WGQ |
| | GGGLVQA | G | PGKERE | GSTYYA | NTVYLQMNSL | EQWYPIYEY | GTLV |
| | GGSLRLS | | GVS | DSVKG | KSEDTAVYYC | DA | TVSS |
| | CAVSGFT | | | | AA | | |
| | LD | | | | | | |
| 64G02/93 | EVQLVES | DYAI | WFRQA | SIRDND | RFIISSDNDKN | VPAGRLRFG | WGQ |
| | GGGLVQA | G | PGKERE | GSTYYA | TVYLQMNSLK | EQWYPIYEY | GTLV |
| | GGSLRLS | | GVS | DSVKG | PEDTAVYYCA | DA | TVSS |
| | CAISGFTL | | | | A | | |
| | V | | | | | | |
| 55A06/94 | EVQLMES | DYAI | WFRQA | SIRDND | RFTISSDNDK | VPAGRLRFG | WGQ |
| | GGGLVQA | G | PGKERE | GSTYYA | NTVNLQMNSL | EQWYPIYEY | GTLV |
| | GGSLRLS | | GVS | DSVKG | KPEDTAVYYC | DA | TVSS |
| | CAASGITL | | | | AA | | |
| | D | | | | | | |
| 64C03/95 | EVQLVES | DYAI | WFRQA | SIRDND | RFTISSDNDK | VPAGRLRY | WGQ |
| | GGGLVQA | G | PGKERE | GSTYYA | NTVYLQMNSL | GEQWYPIYE | GTLV |
| | GGSLRLS | | GVS | DSVKG | KPEDTAVYYC | YDA | TVSS |
| | CAASGFT | | | | AA | | |
| | LD | | | | | | |
| 64G12/96 | EVQLVES | DYAI | WFRQA | SIREND | RFTISSDNDK | VPAGRLRFG | WGQ |
| | GGGLVQA | G | PGKERE | GSTYYA | NTVYLRMNSL | EQWYPIYEY | GTLV |
| | GGSLRLS | | GVS | DSVQG | KPEDTAVYYC | DA | TVSS |
| | CAVSGFT | | | | AA | | |
| | LD | | | | | | |
| 65F03/97 | EVQMVES | DYAI | WFRQA | SIRDND | RFTISSDNDK | VPAGRLRFG | WGQ |
| | GGGLVQA | G | PGKERE | GSTYYA | NTVYLQMNGL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 55F02/98 | EVQLVES | DYAI | WFRQA | SIRDND | RFTISSDNDK | VPAGRLRFG | WGQ |
| | GGGLVQS | G | PGKERE | GSTYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 64E12/99 | EVQLVES | DYAI | WFRQA | SIRDND | RSTISSDNDK | VPAGRLRFG | WGQ |
| | GGGLVQA | G | PGKERE | GSTYYA | NTVYLQMNSL | EQWYPIYEY | GTLV |
| | GGSLRLS | | GVS | DSVKG | KPEDTAVYYC | DA | TVSS |
| | CAVSGFT | | | | AA | | |
| | LD | | | | | | |
| 60C09/100 | EVQLVES | DYAI | WFRQA | SIRDND | RFTISSDNDK | VPAGRLRFG | WGQ |
| | GGGLVQA | G | PGKERE | GSTYYA | NTVYLQMNS | EQWYPIYEY | GILVT |
| | GGSLRLS | | GVS | DSVKW | MKPEDTAVYY | DA | VSS |
| | CAVSGITL | | | | CAA | | |
| | D | | | | | | |
| 64B02/101 | EVQLVES | DYAI | WFRQA | SIRDND | RFTISSDNVK | VPAGRLRFG | WGQ |
| | GGGLVQA | G | PGKERE | GSTYYA | NTVYLQMNSL | EQWYPIYEY | GTLV |
| | GGFLRLT | | GVS | DSVKG | KPEDTAVYYC | DA | TVSS |
| | CAVSGFT | | | | AA | | |
| | LD | | | | | | |
| 64A03/102 | EVQLLES | DYAI | WFRQA | SIRDND | RFTISSDNDK | VPAGRLRIG | WGQ |
| | GGGLVQA | G | PGKERE | GSTYYA | NTVYLQMNSL | EQWYPIYEY | GTLV |
| | GGSLRLS | | GVS | DSVKG | KPEDTAVYYC | DA | TVSS |
| | CAASGFT | | | | AA | | |
| | LD | | | | | | |
| 64C07/103 | EVHLVES | DYAI | WFRQA | SIRDND | RFTISSDNDK | VPAGRLRFG | WGQ |
| | GGGLVQA | G | PGVERE | GSTYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 60A06/104 | EVQLVES | DYAI | WFRQA | SIRDND | RFTISSDNDK | VPAGRLRY | WGQ |
| | GGGLVQA | G | PGKDRE | GSTYYA | NTVYLQMNSL | GEQWYPIYE | GTVV |
| | GGSLRLS | | GVS | DSVKG | KPEDTAVYYC | YDA | TVSS |
| | CAASGFT | | | | AA | | |
| | LD | | | | | | |
| 64B01/105 | EVQLVES | DYAI | WFRQA | SIRDND | RFTISSDNDK | VPAGRLRFG | WGQ |
| | GGGLVQA | G | PGKERE | GSTYYA | NTVYLQMNSL | EQWYPIYEY | GILVT |
| | GGTLRLS | | GVS | DSVKG | KPEDTAVYYC | DA | VSS |
| | CAVSGFT | | | | AA | | |
| | LD | | | | | | |
| 64G01/106 | EVQLVES | DYAI | WFRQA | SIRDND | RFIISSDNDKN | VPAGRLRY | WGQ |
| | GGGLVQA | G | PGKERE | GSTYYA | TVYLQMNNLK | GEQWYPIYE | GTLV |
| | GGSLRLS | | GVS | DSVKG | PEDTAVYYCA | YDA | TVSS |
| | CAASGFT | | | | A | | |
| | LD | | | | | | |
| 56A07/107 | EVQLVES | DYAI | WFRQA | SIRDND | RSTISSDNAR | VPAGRLRFG | WGQ |
| | GGGLVQA | G | PGKERE | GSTYYA | NTVFLQMNSL | EQWYPIYEY | GTLV |
| | GGSLRLS | | GVS | DSVKG | KPEDTAVYYC | DA | TVSS |
| | CAASGFT | | | | AA | | |
| | LD | | | | | | |
| 58D10/108 | EELLVES | DYAI | WFRQA | SIRDND | RFTISSDNDK | VPAGRLRFG | WGQ |
| | GGGSVQA | G | PGKERE | GSTYYA | NTVYLQMNSL | EQWYPIYEY | GTLV |
| | GGSLKLS | | GVS | DSVKG | KPEDTAVYYC | DA | TVSS |
| | CAASGLT | | | | AA | | |
| | LD | | | | | | |
| 65F01/109 | EVQLVES | DYAI | WFRQA | SIRDND | RFTISSDNDK | VPAGRLRFG | WGQ |
| | GGGLVQA | G | PGKERE | GSRYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 53A06/110 | EVQLVES | DYAI | WFRQA | SIRDND | RFTISSDNDK | VPAGRLRFG | WGQ |
| | GGSLVQA | G | PGKERE | GSTYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |
| 55G03/111 | EVQLVES | DYAI | WFRQA | SIRDND | RFTISSDNDK | VPAGRLRFG | WGQ |
| | GGSLVQA | G | PGKERE | GSTYYA | NTVYLQMNSL | EQWYPIYEY | GTLV |
| | GGSLRLS | | GVS | DSVKG | KPEDTAVYYC | DA | TVSS |
| | CAASGLT | | | | AA | | |
| | LD | | | | | | |

TABLE 10-B
Single mutations or combination thereof
yielding improvements in off-rate

| | Fold improvement |
|---|---|
| Mutation(s) | off-rate |
| A24V | 2 |
| F27I | 1.9-2.2 |
| F27L; L100il | 9.4 |
| L29I; L100il | 5.6-6.0 |
| F100bY; L100il | 5.1-9.2 |
| L100il | 3.1-7.0 |

TABLE 11
Amino acid sequence of affinity matured anti-Ang2 VHHs

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00903/112 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGFT | | | | AA | | |
| | LD | | | | | | |
| 00904/113 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGFT | | | | AA | | |
| | LD | | | | | | |
| 00905/114 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGFTI | | | | AA | | |
| | D | | | | | | |
| 00906/115 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGFTI | | | | AA | | |
| | D | | | | | | |
| 00907/116 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGITL | | | | AA | | |
| | D | | | | | | |
| 00908/117 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGITL | | | | AA | | |
| | D | | | | | | |
| 00909/118 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGITI | | | | AA | | |
| | D | | | | | | |
| 00910/119 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGITI | | | | AA | | |
| | D | | | | | | |
| 00911/120 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGLT | | | | AA | | |
| | LD | | | | | | |
| 00912/121 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGLT | | | | AA | | |
| | LD | | | | | | |
| 00913/122 | EVQLVES | DYAI | WFRQA | SIRDNG | RFTISSDNSK | VPAGRLRFG | WGQ |
| | GGGLVQP | G | PGKERE | GSTYYA | NTVYLQMNSL | EQWYPIYEY | GTLV |
| | GGSLRLS | | GVS | DSVKG | RPEDTAVYYC | DA | TVSS |
| | CAASGLTI | | | | AA | | |
| | D | | | | | | |
| 00914/123 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGLTI | | | | AA | | |
| | D | | | | | | |

TABLE 12
Overview of Tₘ, IC₅₀ (pM) in human, mouse and cyno
Ang2 competition ELISA and hAng1/hAng2 IC₅₀ ratios
of affinity matured variants of VHH 00027

| | TSA | | | | |
|---|---|---|---|---|---|
| | Tm @ pH | IC₅₀ in Ang2/Tie2 ELISA | | | |
| | 7.0 | hAng2 | mAng2 | cAng2 | hAng1/hAng2 |
| VHH ID | (° C.) | (pM) | (pM) | (pM) | IC₅₀ ratio |
| 00027 | 61.1 | 672 | 1975 | 728 | >14,878 |
| 00042 | 61.9 | 646 | 1910 | 753 | >15,476 |
| 00903 | 64.0 | 89 | n.d. | n.d. | >112,202 |
| 00904 | 64.4 | 62 | n.d. | n.d. | >162,181 |
| 00905 | 64.0 | 79 | n.d. | n.d. | >125,893 |
| 00906 | 64.4 | 45 | n.d. | n.d. | >223,872 |
| 00907 | 67.3 | 62 | n.d. | n.d. | >162,181 |
| 00908 | 67.3 | 45 | 85 | 79 | >192,014 |
| 00909 | 66.1 | 53 | n.d. | n.d. | >190,546 |
| 00910 | 66.1 | 42 | n.d. | n.d. | >239,883 |
| 00911 | 66.1 | 59 | n.d. | n.d. | >169,824 |
| 00912 | 66.1 | 62 | n.d. | n.d. | >162,181 |
| 00913 | 64.0 | 42 | n.d. | n.d. | >239,883 |
| 00914 | 64.4 | 37 | n.d. | n.d. | >269,153 |

From potency, Tₘ and sequence perspective VHH 00908 is taken forward into a second cycle of combined affinity maturation and sequence optimization (Example 7.3).

### Example 7

### Sequence Optimization of Selected VHH 1D01, 28D10 and 37F02

### 7.1 VHH 1D01

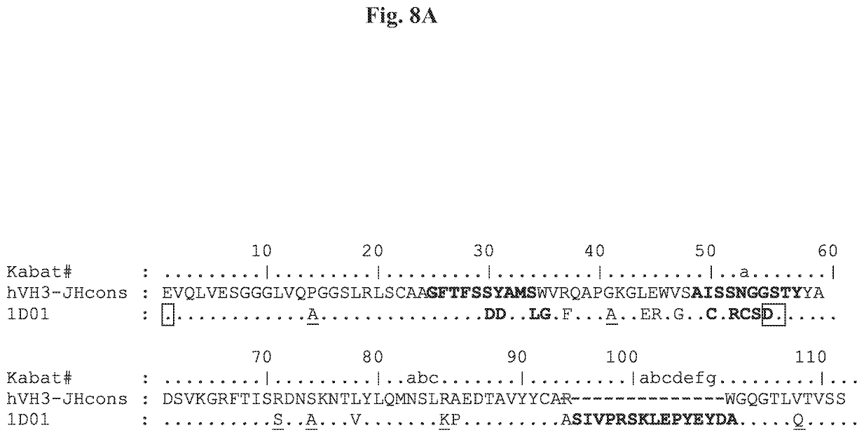

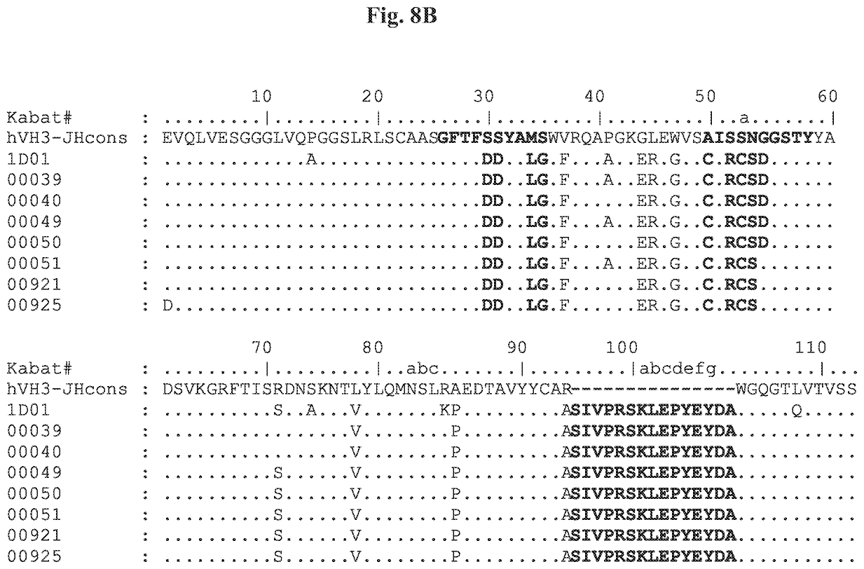

The amino acid sequence of anti-Ang2 VHH 1D01 (see FIG. 8-A) is aligned to the human germline VH3/JH consensus sequence. Residues are numbered according to Kabat, CDRs are shown in grey according to AbM definition (Oxford Molecular's AbM antibody modeling software). Residues to be mutated to their human counterpart are underlined. Potential post-translational modification sites to be tackled are boxed. The alignment shows that 1 D01 contains 6 framework mutations relative to the reference germline sequence. Non-human residues at positions 14, 41, 71, 74, 83 and 108 are selected for substitution with their human germline counterpart. A set of seven 1 D01 variants carrying different combinations of human residues on these positions (FIG. 8-B) is constructed and produced (Example 5). In parallel, in 3 of these 7 variants a potential Asp isomerization site at position D₅₄G₅₅ is removed by introducing a D₅₄G substitution, and in 1 of these 7 variants a potential pyroGlu formation site at position E₁ is removed by an E₁D substitution (AA sequences are listed in Table 15).

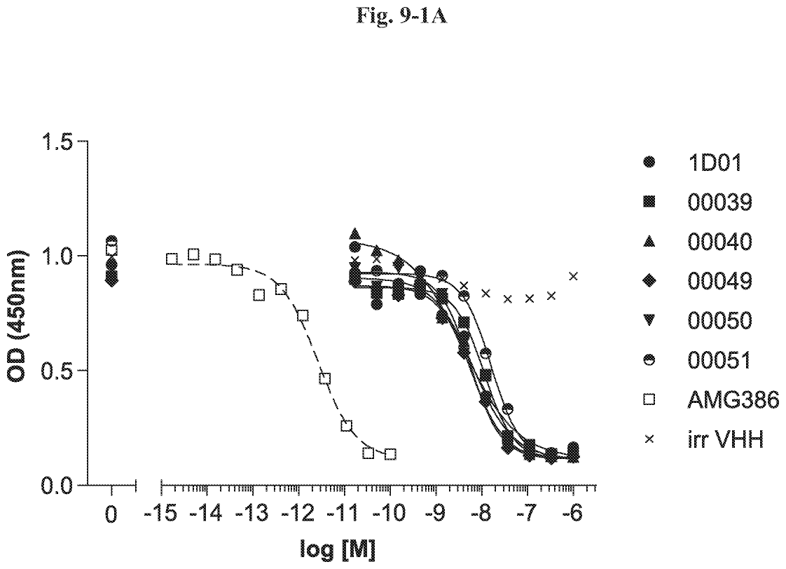

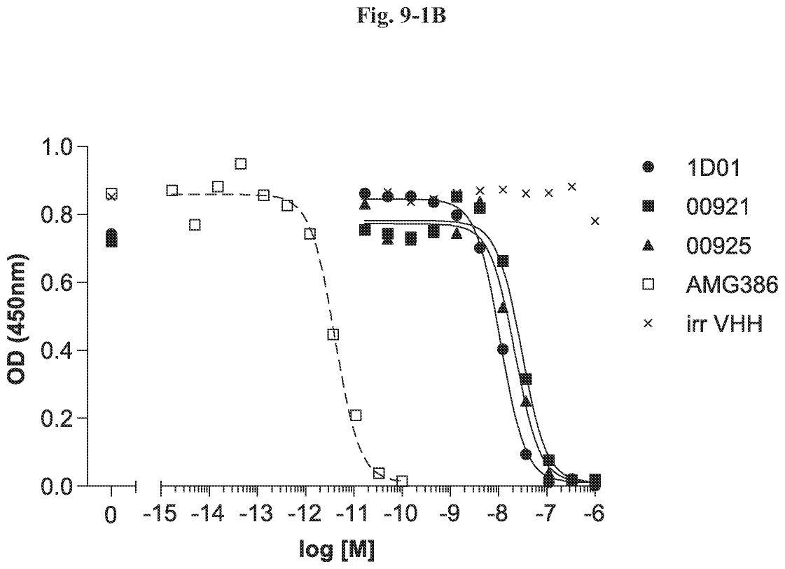

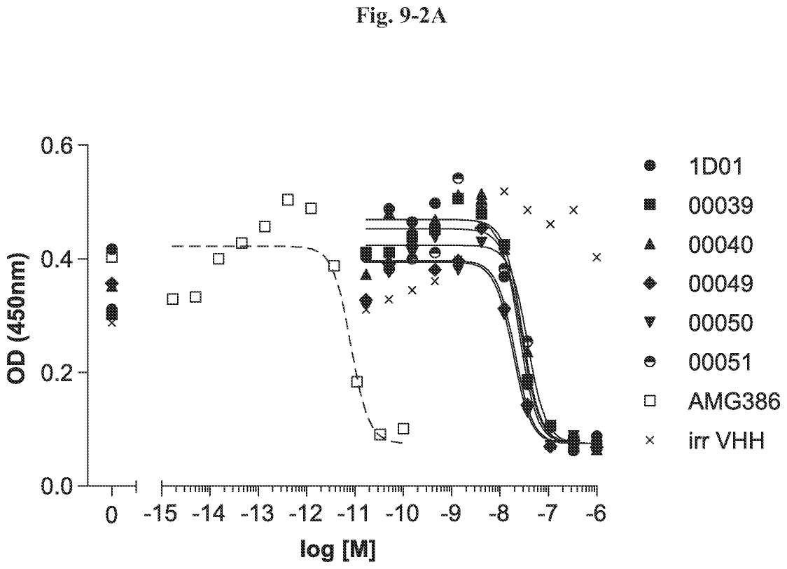

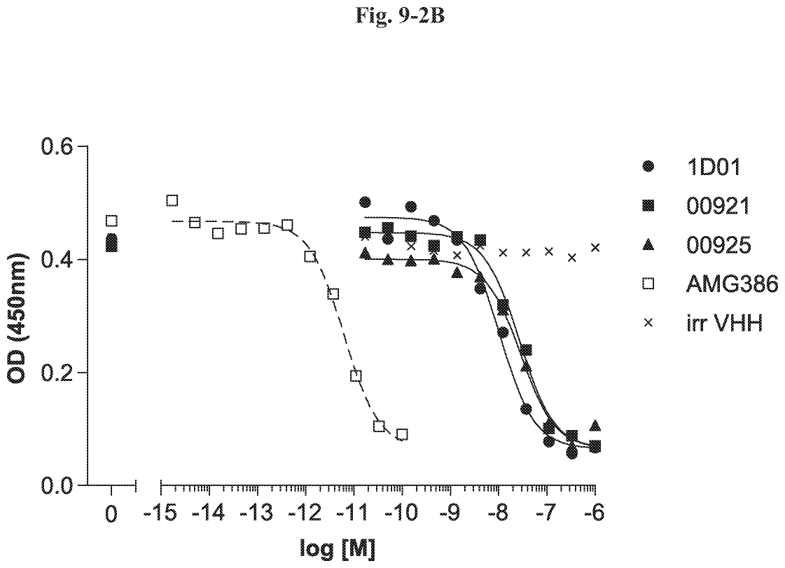

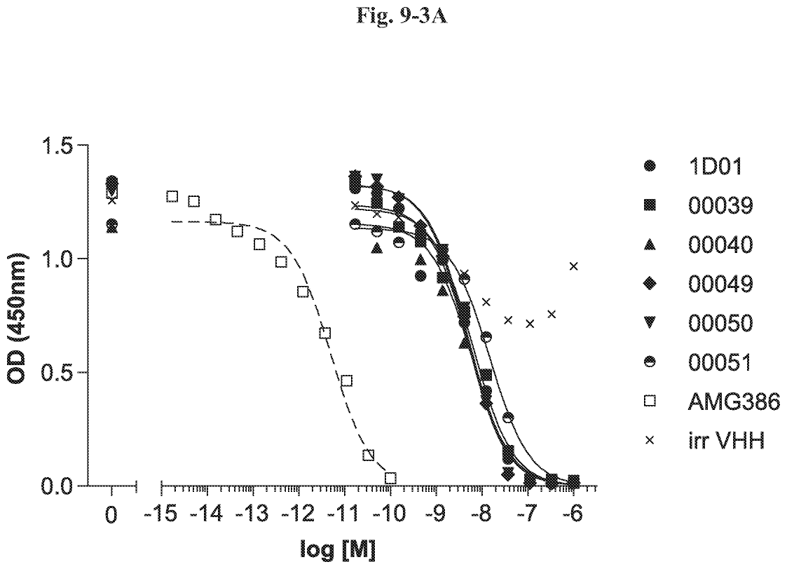

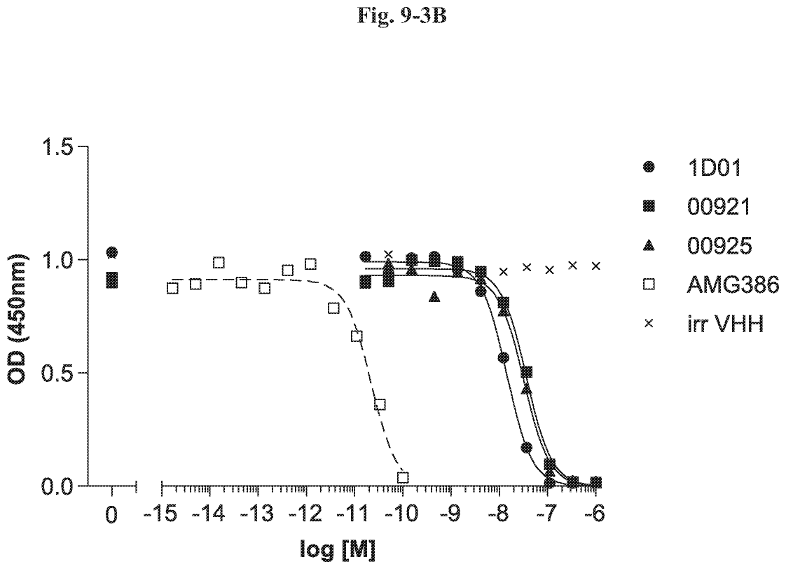

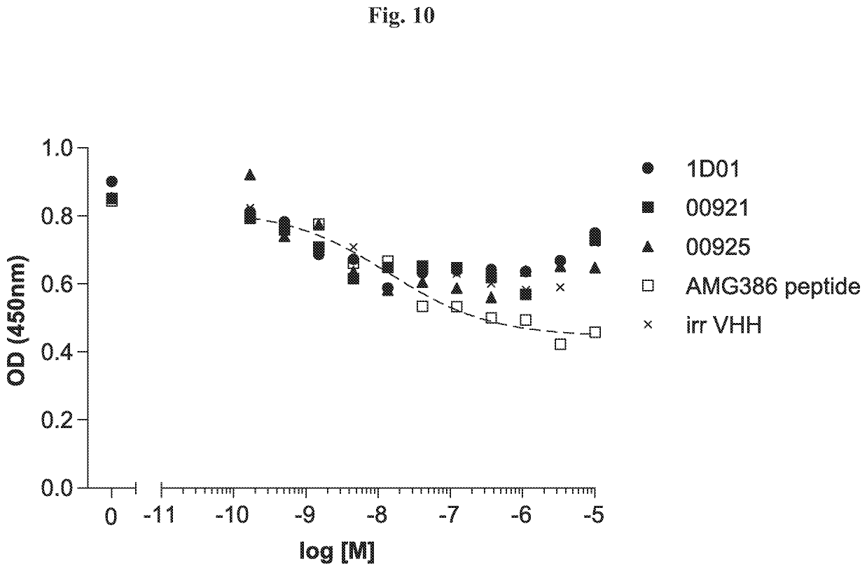

These variants are characterized as purified protein in the human (FIG. 9-1), mouse (FIG. 9-2) and cyno (FIG. 9-3) Ang2/Tie2 competition ELISA (Example 5.1; Example 5.2), the hAng1/hTie2 competition ELISA (Example 5.3; FIG. 10). Additionally, melting temperature (Tₘ) of each variant is determined in thermal shift assay (Example 6). An overview of the data can be found in Table 13. Additionally, % FR identity to the human germline is calculated according to AbM definition (Oxford Molecular's AbM antibody modeling software). Affinity of VHH 00921 for human, cyno, mouse and rat Ang2 is shown in Table 14 (Example 5.4)

TABLE 13
Overview of Tₘ, IC₅₀ (nM) in human, mouse and cyno Ang2 competition
ELISA and hAng1/hAng2 IC₅₀ ratios of sequence optimized variants
of VHH 1D01

| | TSA | IC₅₀ in Ang2-Tie2 ELISA | | | hAng1/ | % FR |
|---|---|---|---|---|---|---|
| VHH | Tₘ (° C.) | hAng2 | mAng2 | cAng2 | hAng2 | identity |
| ID | @ pH7 | (nM) | (nM) | (nM) | IC₅₀ ratio | AbM |
| 1D01 | 65.0 | 6.6 | 12.4 | 9.1 | >1,511 | 85.4 |
| 00039 | 61.5 | 11.9 | 28.1 | 6.4 | n.d | 91.0 |
| 00040 | 64.0 | 6.4 | 29.0 | 5.8 | n.d | 92.1 |
| 00049 | 66.1 | 6.9 | 20.3 | 4.5 | n.d | 89.9 |
| 00050 | 67.7 | 6.0 | 18.6 | 4.8 | n.d | 91.0 |
| 00051 | 64.8 | 16.1 | 37.9 | 15 | n.d | 89.9 |
| 00921 | 67.3 | 30.4 | 27.0 | 37 | >329 | 91.0 |
| 00925 | 66.5 | 22.1 | 28.4 | 33 | >453 | 89.9 |

n.d., not determined

TABLE 14
Affinity KD of purified VHH 00921 for recombinant human,
cyno, mouse and rat Ang2

| | human Ang2-FLD | | | cyno Ang2-FLD | | |
|---|---|---|---|---|---|---|
| | kₐ | k_{d} | K_{D} | kₐ | k_{d} | K_{D} |
| | (1/Ms) | (1/s) | (M) | (1/Ms) | (1/s) | (M) |
| 00921 | 4.0E+06 | 2.7E−02 | 6.6E−09 | 1.1E+06 | 2.5E−02 | 2.3E−09 |
| | mouse Ang2-FLD | | | rat Ang2-FLD | | |
| | kₐ | k_{d} | K_{D} | kₐ | k_{d} | K_{D} |
| | (1/Ms) | (1/s) | (M) | (1/Ms) | (1/s) | (M) |
| 00921 | 3.3E+06 | 2.5E−02 | 7.5E−09 | 1.2E+06 | 4.9E−02 | 4.2E−08 |

TABLE 15
Amino acid sequence of sequence optimized variants of anti-Ang2
VHH 1D01

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00039/124 | EVQLVES | DYAL | WFRQA | CIRCSD | RFTISRDNSK | SIVPRSKLE | WGQ |
| | GGGLVQP | G | AGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00040/125 | EVQLVES | DYAL | WFRQA | CIRCSD | RFTISRDNSK | SIVPRSKLE | WGQ |
| | GGGLVQP | G | PGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00049/126 | EVQLVES | DYAL | WFRQA | CIRCSD | RFTISSDNSK | SIVPRSKLE | WGQ |
| | GGGLVQP | G | AGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00050/127 | EVQLVES | DYAL | WFRQA | CIRCSD | RFTISSDNSK | SIVPRSKLE | WGQ |
| | GGGLVQP | G | PGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00051/128 | EVQLVES | DYAL | WFRQA | CIRCSG | RFTISSDNSK | SIVPRSKLE | WGQ |
| | GGGLVQP | G | AGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00921/129 | EVQLVES | DYAL | WFRQA | CIRCSG | RFTISSDNSK | SIVPRSKLE | WGQ |
| | GGGLVQP | G | PGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |
| 00925/130 | DVQLVES | DYAL | WFRQA | CIRCSG | RFTISSDNSK | SIVPRSKLE | WGQ |
| | GGGLVQP | G | PGKERE | GSTYYA | NTVYLQMNSL | PYEYDA | GTLV |
| | GGSLRLS | | GVS | DSVKG | RPEDTAVYYC | | TVSS |
| | CAASGFT | | | | AA | | |
| | FD | | | | | | |

### 7.2 VHH 37F02

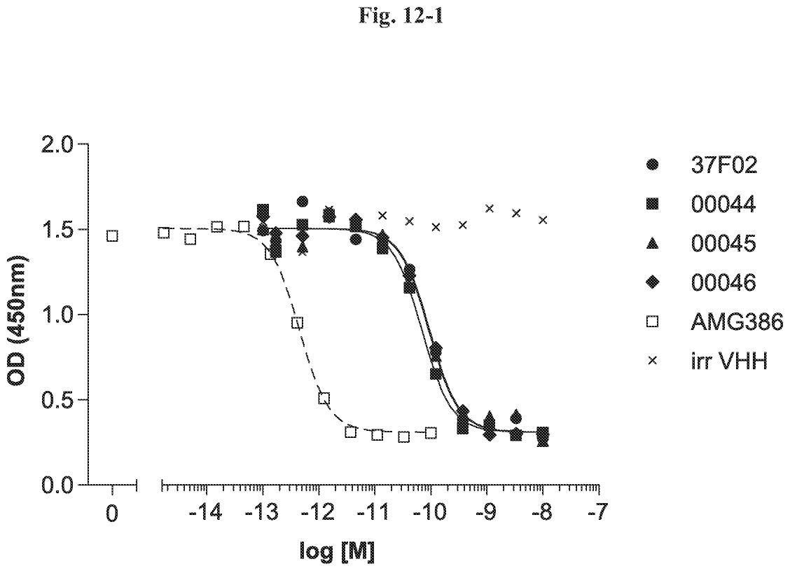

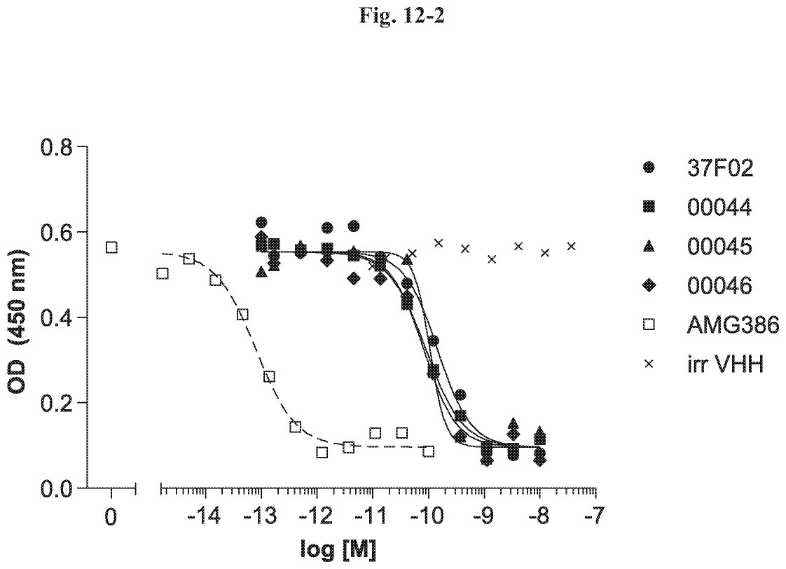

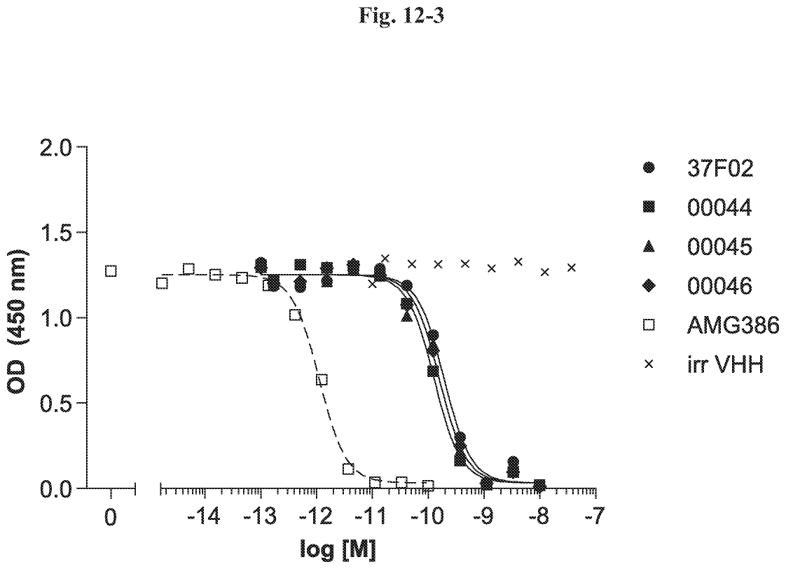

The amino acid sequence of anti-Ang2 VHH 37F02 (see FIG. 12-A) is aligned to the human germline VH3/JH consensus sequence. Residues are numbered according to Kabat, CDRs are shown in grey according to AbM definition (Oxford Molecular's AbM antibody modeling software). Residues to be mutated to their human counterpart are underlined. Potential post-translational modification sites to be tackled are boxed. The alignment shows that 37F02 contains 4 framework mutations relative to the reference germline sequence. Non-human residues at positions 60, 74, 83 and 108 are selected for substitution with their human germline counterpart. In parallel, a potential Asp isomerization site at position D₅₄G₅₅ is removed by introducing a D₅₄G substitution. A set of three cycle 1 37F02 variants carrying different combinations of human residues on these positions (FIG. 12-B) is constructed and produced (Example 5; AA sequences are listed in Table 21-1).

These variants are characterized as purified protein in the human (FIG. 12-1), mouse (FIG. 12-2) and cyno (FIG. 12-3) Ang2/Tie2 competition ELISA (Example 5.1; Example 5.2). Additionally, melting temperature (Tₘ) of each variant is determined in thermal shift assay (Example 6). An overview of the data can be found in Table 16. Additionally, % FR identity to the human germline is calculated according to AbM definition (Oxford Molecular's AbM antibody modeling software).

TABLE 16
Overview of Tₘ, IC₅₀ (pM) in human, mouse and cyno Ang2
competition ELISA and hAng1/hAng2 IC₅₀ ratios of cycle 1
sequence optimized variants of VHH 37F02

| | TSA | IC50 in Ang2-Tie2 ELISA | | | | % FR |
|---|---|---|---|---|---|---|
| VHH | Tm (° C.) | hAng2 | mAng2 | cAng2 | hAng1/hAng2 | identity |
| ID | @ pH7 | (pM) | (pM) | (pM) | IC₅₀ ratio | AbM |
| 37F02 | 66.1 | 77 | 110 | 150 | >130,317 | 87.6 |
| 00044 | 66.9 | 69 | 91 | 130 | n.d. | 91.0 |
| 00045 | 71.1 | 120 | 110 | 160 | n.d. | 92.1 |
| 00046 | 69.8 | 95 | 83 | 160 | n.d. | 91.0 |

A NNK library approach is used to knock out two potential post-translational modifications sites in CDR3: i) oxidation sensitive Met on position 100e and ii) Asp isomerization site on position D₉₅S₉₆. Since D₅₄G is tolerated (VHH 00046 and 00920; Table 16 and Table 17) no NNK approach was used to knock out this potential Asp isomerization site.

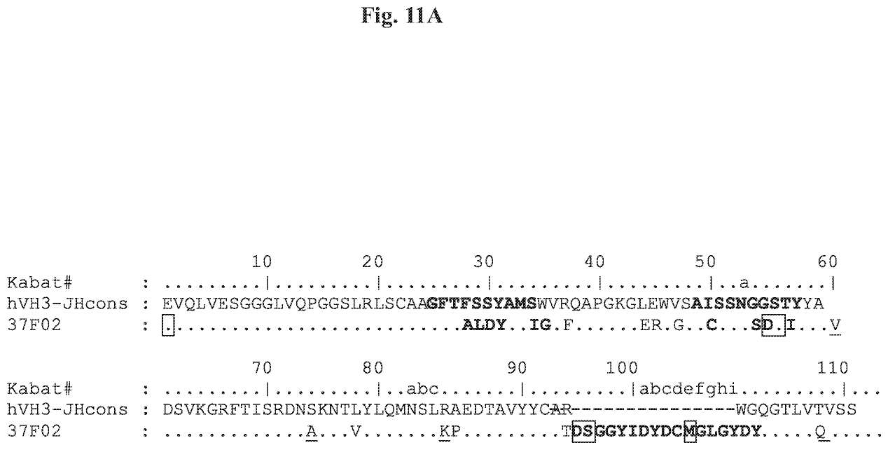

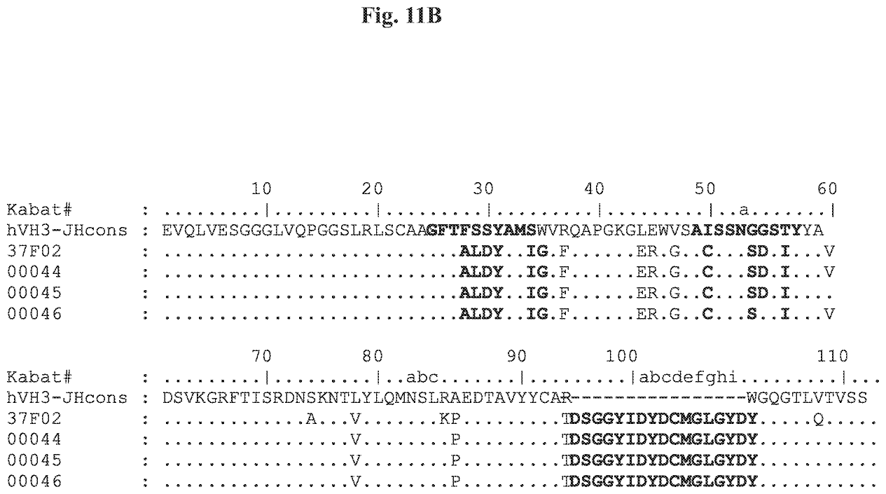

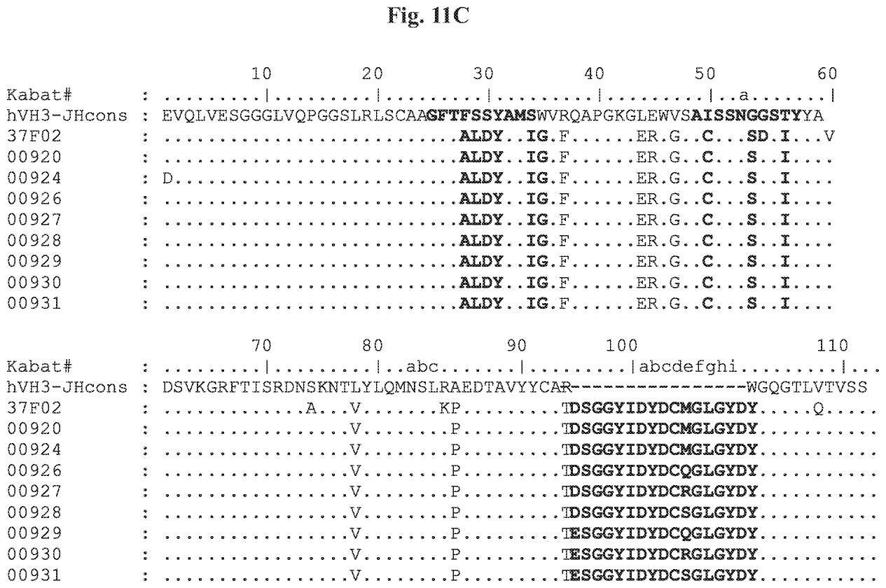

In the end, 3 NNK libraries containing VHH clones carrying substitutions at positions D₉₅, S₉₆ and M₁₀₀ₑ to all other amino acids are screened in a hAng2/hTie2 competition AlphaScreen assay (Example 2). Briefly, periplasmic extracts containing expressed VHH are screened at 3 different dilutions (corresponding roughly to EC₂₀, EC₅₀ and EC₈₀ of the parental VHH 37F02) and changes in % inhibition at the different dilution points are compared to parental 37F02. Based on the screening results and the data shown in Table 17, 8 additional cycle 2 VHH variants are constructed (based on VHH 00920 backbone) carrying different knock-out combinations of D₉₅S₉₆ and M₁₀₀ₑ (FIG. 11-C; AA sequences are listed in Table 19-2).

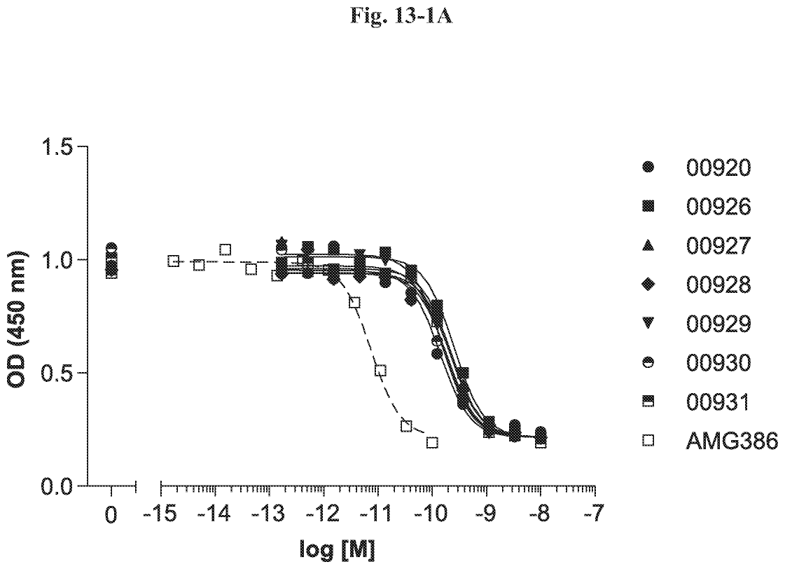

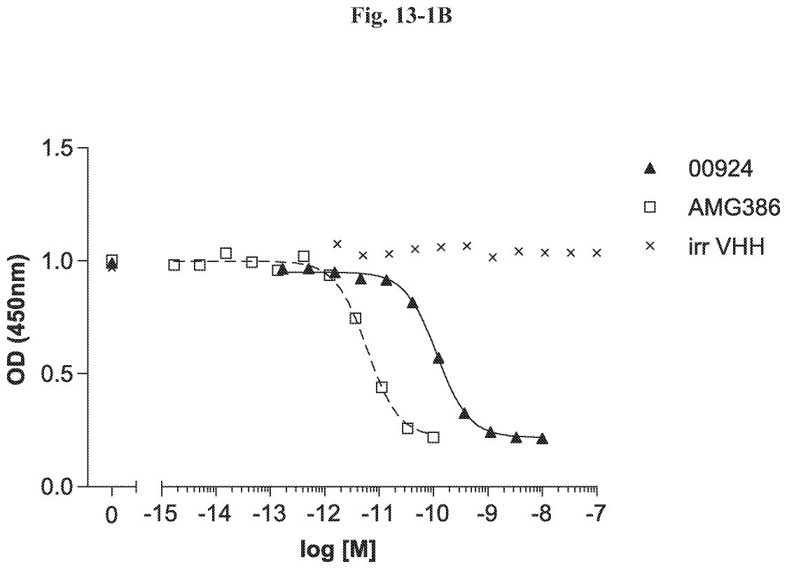

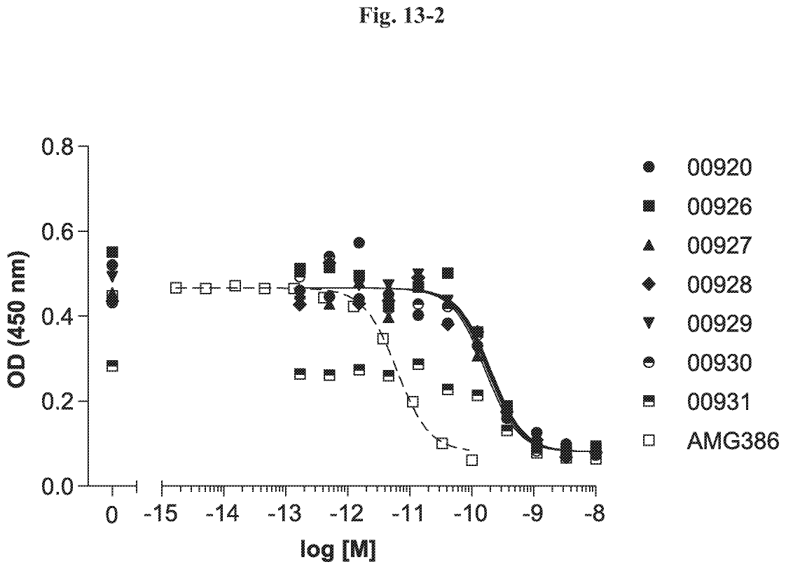

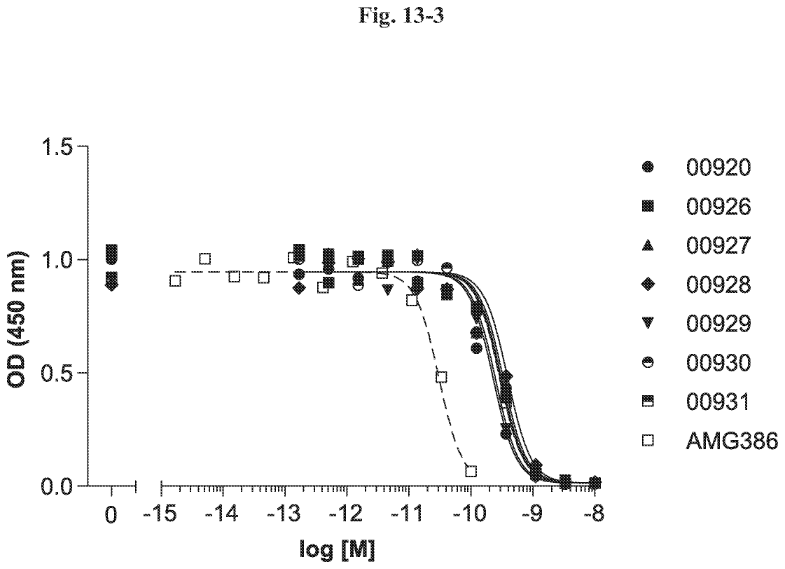

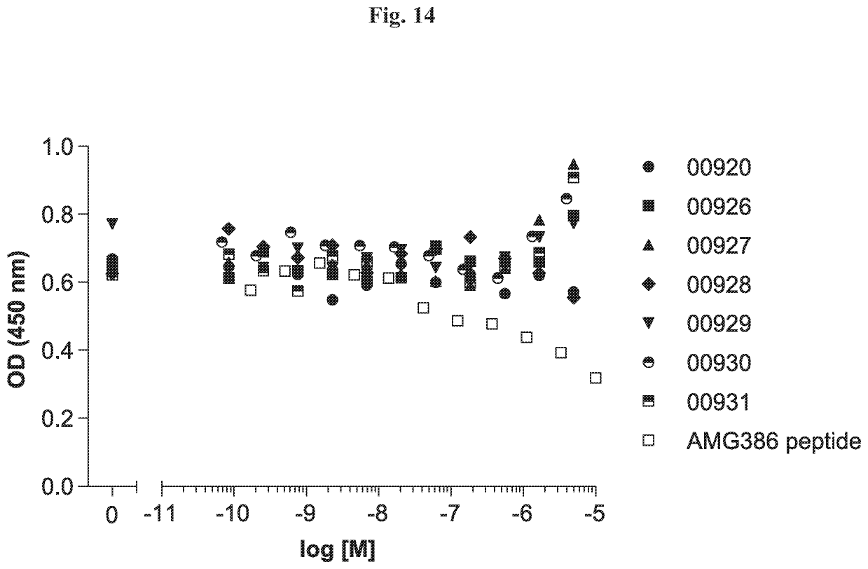

All these variants are characterized as purified protein in the human (FIG. 13-1), mouse (FIG. 12-2) and cyno (FIG. 12-3) Ang2/Tie2 competition ELISA (Example 5.1; Example 5.2), the hAng1/hTie2 competition ELISA (Example 5.3; FIG. 14). Additionally, melting temperature (Tₘ) of each variant is determined in thermal shift assay (Example 6). An overview of the data can be found in Table 17. Additionally, % FR identity to the human germline is calculated. Affinity of VHH 00928 for human, mouse, cyno and rat Ang2 is shown in Table 18.

TABLE 17
Overview of Tₘ, IC₅₀ (pM) in human, mouse and cyno Ang2
competition ELISA and hAng1/hAng2 IC₅₀ ratios of cycle 2
sequence optimized variants of VHH 37F02

| | TSA | IC50 in Ang2-Tie2 ELISA | | | hAng1/ | % FR |
|---|---|---|---|---|---|---|
| | Tm (° C.) | hAng2 | mAng2 | cAng2 | hAng2 | identity |
| VHH ID | @ pH7 | (pM) | (pM) | (pM) | ratio | AbM |
| 37F02 | 66.1 | 77 | 110 | 150 | >130,317 | 87.6 |
| 00920 | 73.6 | 130 | 150 | 230 | >79,159 | 92.1 |
| 00924 | 73.1 | 110 | n.d. | n.d. | n.d. | 91.0 |
| 00926 | 71.5 | 250 | 200 | 290 | >39,446 | 92.1 |
| 00927 | 73.6 | 220 | 190 | 330 | >44,668 | 92.1 |
| 00928 | 72.7 | 220 | 200 | 390 | >45,604 | 92.1 |
| 00929 | 69.0 | 150 | 150 | 250 | >65,013 | 92.1 |
| 00930 | 69.8 | 190 | 170 | 310 | >53,580 | 92.1 |
| 00931 | 69.4 | 170 | 150 | 290 | >57,677 | 92.1 |

n.d., not determined

TABLE 18
Affinity KD of purified VHH 00928 for recombinant human,
cyno, mouse and rat Ang2

| | human Ang2-FLD | | | cyno Ang2-FLD | | |
|---|---|---|---|---|---|---|
| | kₐ | k_{d} | K_{D} | kₐ | k_{d} | K_{D} |
| | (1/Ms) | (1/s) | (M) | (1/Ms) | (1/s) | (M) |
| 00928 | 6.2+05 | 4.0E−05 | 6.4E−11 | 9.7E+05 | 4.9E−05 | 5.0E−11 |
| | mouse Ang2-FLD | | | rat Ang2-FLD | | |
| | kₐ | k_{d} | K_{D} | kₐ | k_{d} | K_{D} |
| | (1/Ms) | (1/s) | (M) | (1/Ms) | (1/s) | (M) |
| 00928 | 4.2E+05 | 7.3E−05 | 1.7E−10 | 1.8E+05 | 5.4E−05 | 2.9E−10 |

TABLE 19-1

| VHH ID/ | | | | | | | |
|---|---|---|---|---|---|---|---|
| SEQ ID | | | | | | | |
| NOs: | FR1 | CDR1 | FR2 | CDR2 | FR3 | CDR3 | FR4 |
| 00044/131 | EVQLVES | YYAI | WFRQA | CISSSD | RFTISRDNSK | DSGGYIDYD | WGQ |
| | GGGLVQP | G | PGKERE | GITYYV | NTVYLQMNSL | CMGLGYDY | GTLV |
| | GGSLRLS | | GVS | DSVKG | RPEDTAVYYC | | TVSS |
| | CAASGFA | | | | AT | | |
| | LD | | | | | | |
| 00045/132 | EVQLVES | YYAI | WFRQA | CISSSD | RFTISRDNSK | DSGGYIDYD | WGQ |
| | GGGLVQP | G | PGKERE | GITYYA | NTVYLQMNSL | CMGLGYDY | GTLV |
| | GGSLRLS | | GVS | DSVKG | RPEDTAVYYC | | TVSS |
| | CAASGFA | | | | AT | | |
| | LD | | | | | | |
| 00046/133 | EVQLVES | YYAI | WFRQA | CISSSG | RFTISRDNSK | DSGGYIDYD | WGQ |
| | GGGLVQP | G | PGKERE | GITYYV | NTVYLQMNSL | CMGLGYDY | GTLV |
| | GGSLRLS | | GVS | DSVKG | RPEDTAVYYC | | TVSS |
| | CAASGFA | | | | AT | | |
| | LD | | | | | | |

TABLE 19-2

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00920/134 | EVQLVES | YYAI | WFRQA | CISSSG | RFTISRDNSK | DSGGYIDYD | WGQ |
| | GGGLVQP | G | PGKERE | GITYYA | NTVYLQMNSL | CMGLGYDY | GTLV |
| | GGSLRLS | | GVS | DSVKG | RPEDTAVYYC | | TVSS |
| | CAASGFA | | | | AT | | |
| | LD | | | | | | |
| 00924/135 | DVQLVES | YYAI | WFRQA | CISSSG | RFTISRDNSK | DSGGYIDYD | WGQ |
| | GGGLVQP | G | PGKERE | GITYYA | NTVYLQMNSL | CMGLGYDY | GTLV |
| | GGSLRLS | | GVS | DSVKG | RPEDTAVYYC | | TVSS |
| | CAASGFA | | | | AT | | |
| | LD | | | | | | |
| 00926/136 | EVQLVES | YYAI | WFRQA | CISSSG | RFTISRDNSK | DSGGYIDYD | WGQ |
| | GGGLVQP | G | PGKERE | GITYYA | NTVYLQMNSL | CQGLGYDY | GTLV |
| | GGSLRLS | | GVS | DSVKG | RPEDTAVYYC | | TVSS |
| | CAASGFA | | | | AT | | |
| | LD | | | | | | |
| 00927/137 | EVQLVES | YYAI | WFRQA | CISSSG | RFTISRDNSK | DSGGYIDYD | WGQ |
| | GGGLVQP | G | PGKERE | GITYYA | NTVYLQMNSL | CRGLGYDY | GTLV |
| | GGSLRLS | | GVS | DSVKG | RPEDTAVYYC | | TVSS |
| | CAASGFA | | | | AT | | |
| | LD | | | | | | |
| 00928/138 | EVQLVES | YYAI | WFRQA | CISSSG | RFTISRDNSK | DSGGYIDYD | WGQ |
| | GGGLVQP | G | PGKERE | GITYYA | NTVYLQMNSL | CSGLGYDY | GTLV |
| | GGSLRLS | | GVS | DSVKG | RPEDTAVYYC | | TVSS |
| | CAASGFA | | | | AT | | |
| | LD | | | | | | |
| 00929/139 | EVQLVES | YYAI | WFRQA | CISSSG | RFTISRDNSK | ESGGYIDYD | WGQ |
| | GGGLVQP | G | PGKERE | GITYYA | NTVYLQMNSL | CQGLGYDY | GTLV |
| | GGSLRLS | | GVS | DSVKG | RPEDTAVYYC | | TVSS |
| | CAASGFA | | | | AT | | |
| | LD | | | | | | |
| 00930/140 | EVQLVES | YYAI | WFRQA | CISSSG | RFTISRDNSK | ESGGYIDYD | WGQ |
| | GGGLVQP | G | PGKERE | GITYYA | NTVYLQMNSL | CRGLGYDY | GTLV |
| | GGSLRLS | | GVS | DSVKG | RPEDTAVYYC | | TVSS |
| | CAASGFA | | | | AT | | |
| | LD | | | | | | |
| 00931/141 | EVQLVES | YYAI | WFRQA | CISSSG | RFTISRDNSK | ESGGYIDYD | WGQ |
| | GGGLVQP | G | PGKERE | GITYYA | NTVYLQMNSL | CSGLGYDY | GTLV |
| | GGSLRLS | | GVS | DSVKG | RPEDTAVYYC | | TVSS |
| | CAASGFA | | | | AT | | |
| | LD | | | | | | |

### 7.3 VHH 28D10

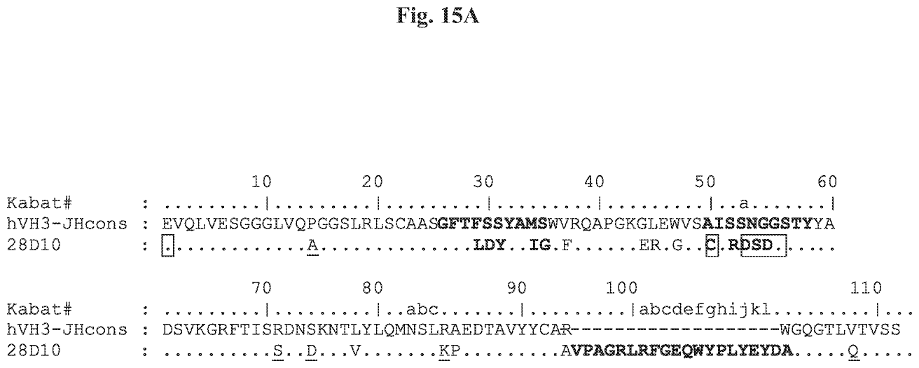

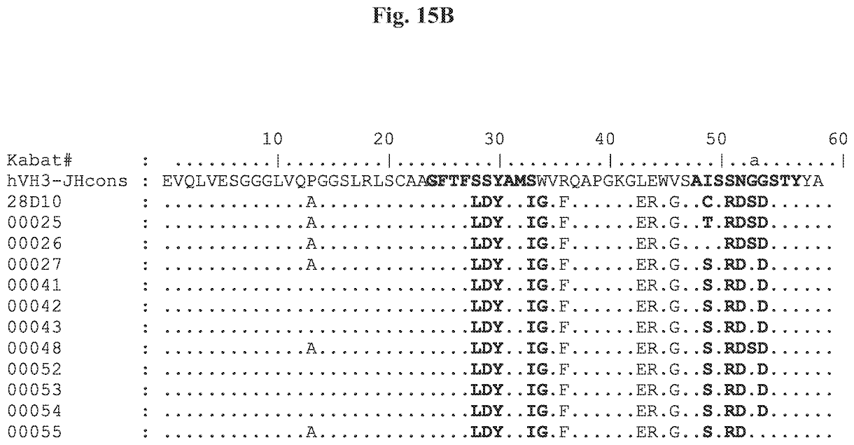

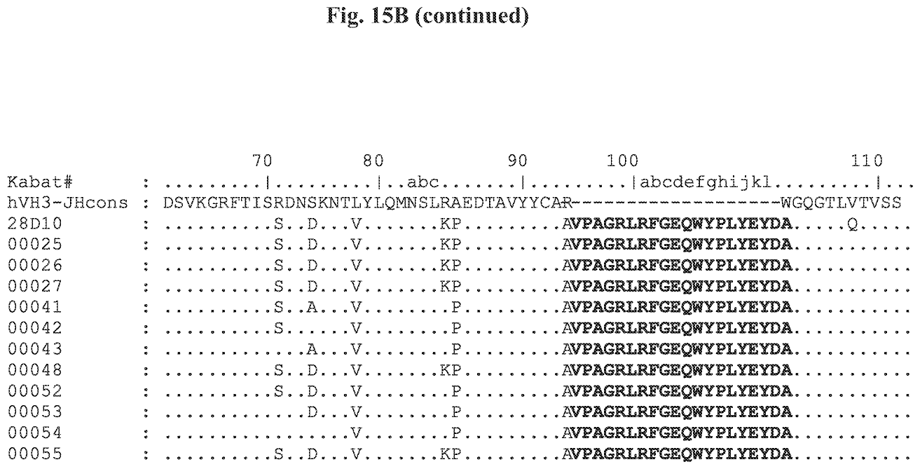

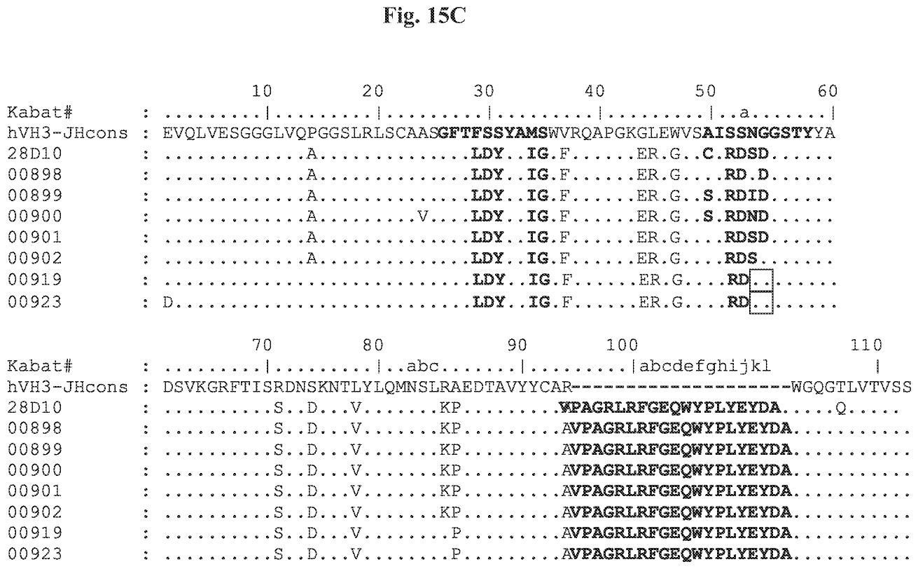

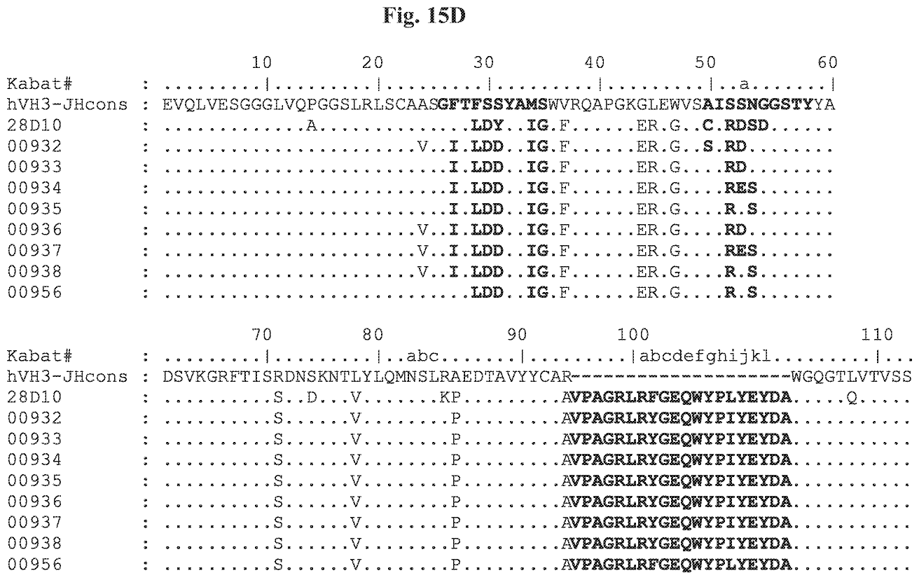

The amino acid sequence of anti-Ang2 VHH 28D10 (see FIG. 15-A) is aligned to the human germline VH3/JH consensus sequence. Residues are numbered according to Kabat, CDRs are shown in grey according to AbM definition (Oxford Molecular's AbM antibody modeling software). Residues to be mutated to their human counterpart are underlined. Potential post-translational modification sites to be tackled are boxed. The alignment shows that 28D10 contains 5 framework mutations relative to the reference germline sequence. Non-human residues at positions 14, 71, 74, 83 and 108 are selected for substitution with their human germline counterpart. In parallel, a potential Asp isomerization site at position D₅₄G₅₅ is removed by introducing a D₅₄G substitution. The free cystein at position 50 was removed by substitution with Ala, Thr or Ser. In the end, a set of eleven cycle 1 28D10 variants carrying different combinations of human residues on these positions is constructed and produced (see FIG. 15-B; AA sequences are listed in Table 24-1).

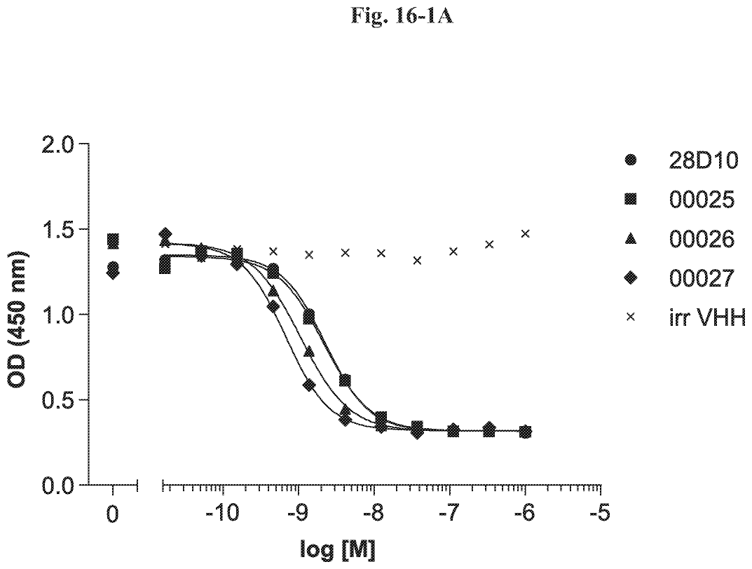

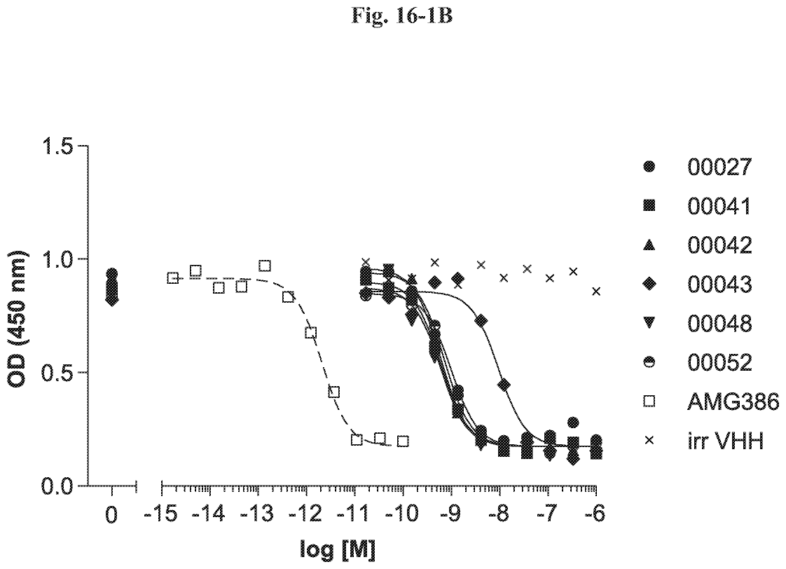

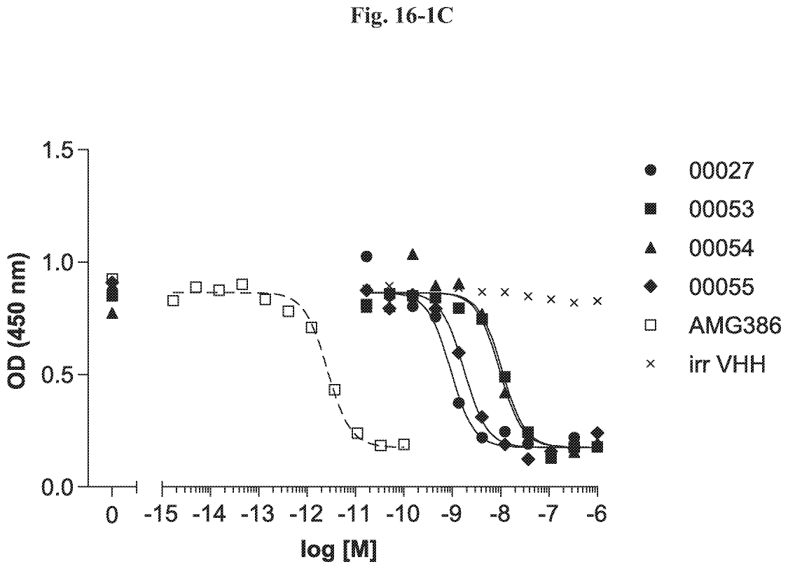

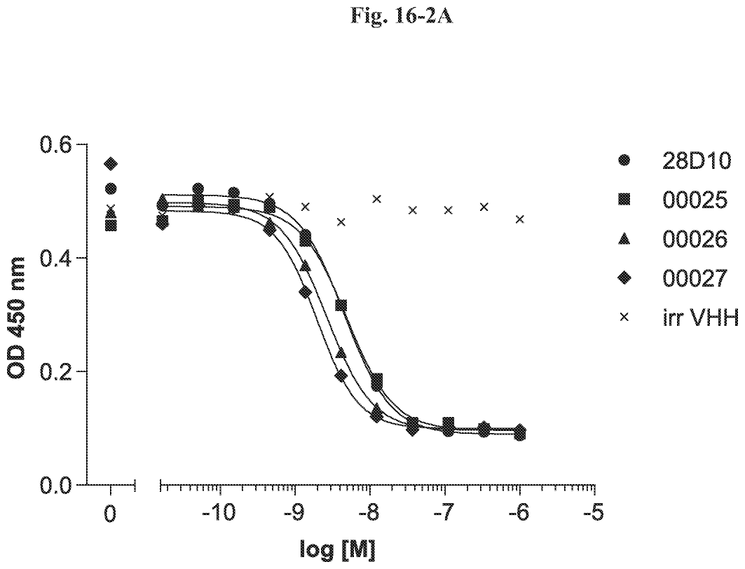

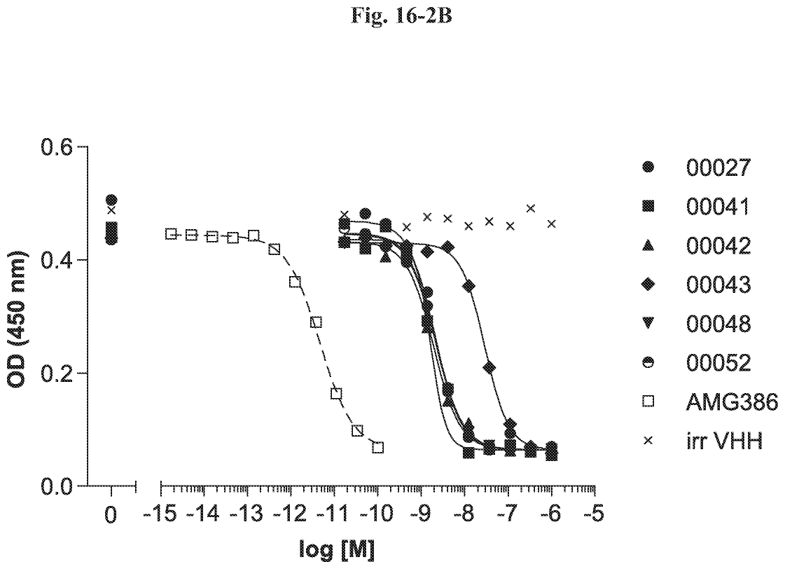

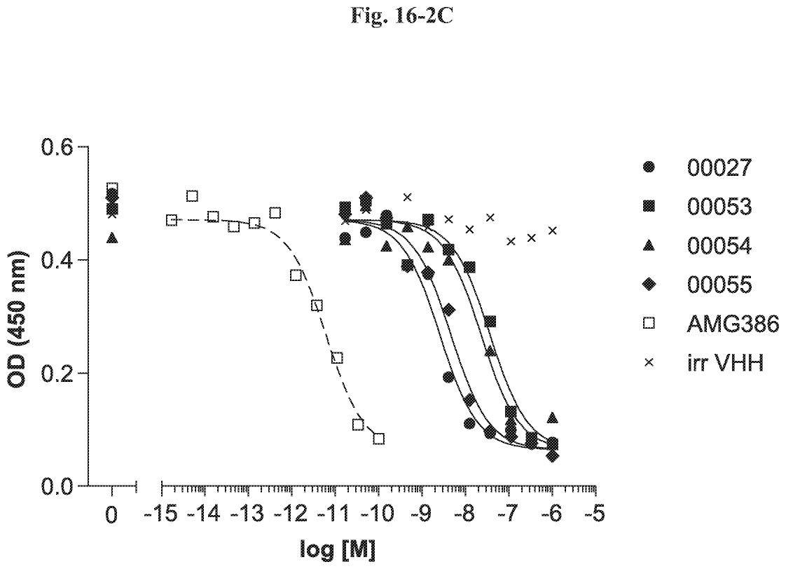

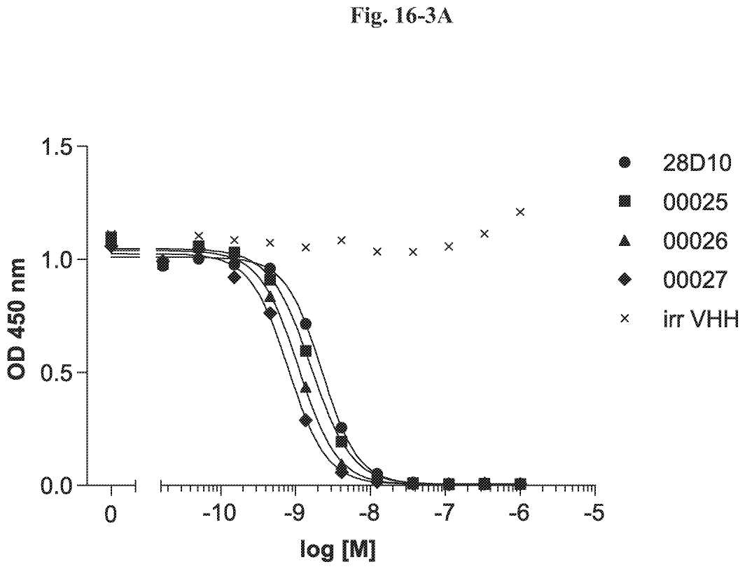

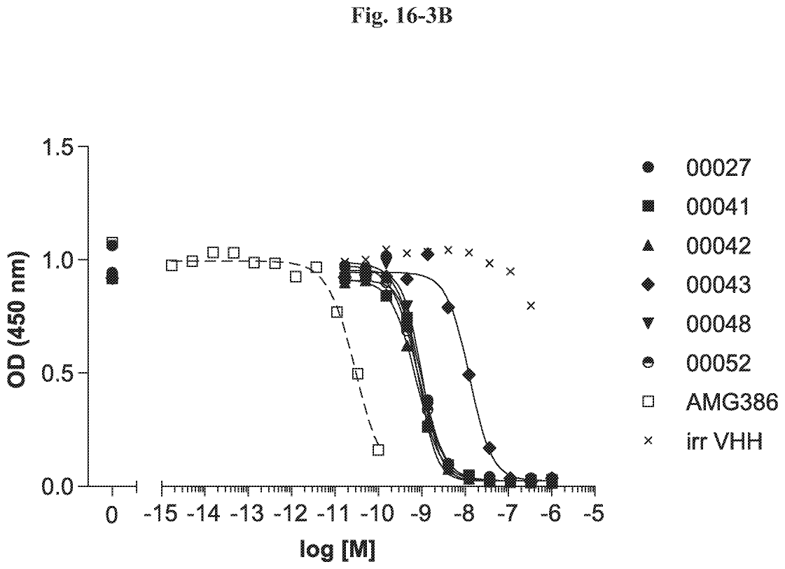

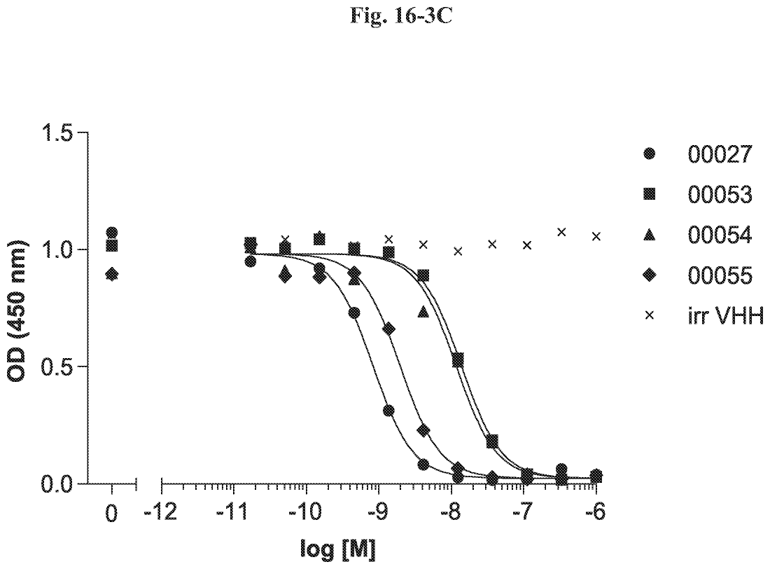

These variants are characterized as purified protein in the human (FIG. 16-1), mouse (FIG. 16-2) and cyno (FIG. 16-3) Ang2/Tie2 competition ELISA (Example 5.1; Example 5.2), the hAng1/hTie2 competition ELISA (Example 5.3; FIG. 17). Additionally, melting temperature (Tₘ) of each variant is determined in thermal shift assay (Example 6). An overview of the data can be found in Table 20. Additionally, % FR identity to the human germline is calculated.

TABLE 20
Overview of Tₘ, IC₅₀ (nM) in human, mouse and cyno Ang2
competition ELISA and hAng1/hAng2 IC₅₀ ratios of cycle 1
sequence optimized variants of VHH 28D10

| | TSA | IC50 in Ang2-Tie2 ELISA | | | hAng1/ | % FR |
|---|---|---|---|---|---|---|
| VHH ID | Tm (° C.) | hAng2 | mAng2 | cAng2 | hAng2 | identity |
| | @ pH7 | (nM) | (nM) | (nM) | ratio | AbM |
| 28D10 | 66.1 | 1.8 | 5.7 | 2.1 | >5,675 | 86.5 |
| 00025 | 63.2 | 2.1 | 4.9 | 1.7 | n.d. | 87.6 |
| 00026 | 68.2 | 1.0 | 2.6 | 1.1 | n.d. | 87.6 |
| 00027 | 61.1 | 0.7 | 2.0 | 0.7 | >14,878 | 87.6 |
| 00041 | 62.3 | 0.6 | 1.7 | 0.9 | n.d. | 89.9 |
| 00042 | 61.9 | 0.7 | 1.9 | 0.8 | >15,476 | 91.0 |
| 00043 | 65.7 | 9.5 | 29.2 | 12.6 | n.d. | 91.0 |
| 00048 | 63.2 | 0.6 | 2.1 | 1.0 | n.d. | 87.6 |
| 00052 | 61.1 | 1.0 | 2.1 | 0.8 | n.d. | 89.9 |
| 00053 | 64.8 | 10.7 | 36.7 | 13.8 | n.d. | 91.0 |
| 00054 | 65.7 | 9.5 | 22.9 | 11.7 | n.d. | 92.1 |
| 00055 | 63.6 | 1.8 | 4.6 | 2.0 | n.d. | 87.6 |

n.d., not determined

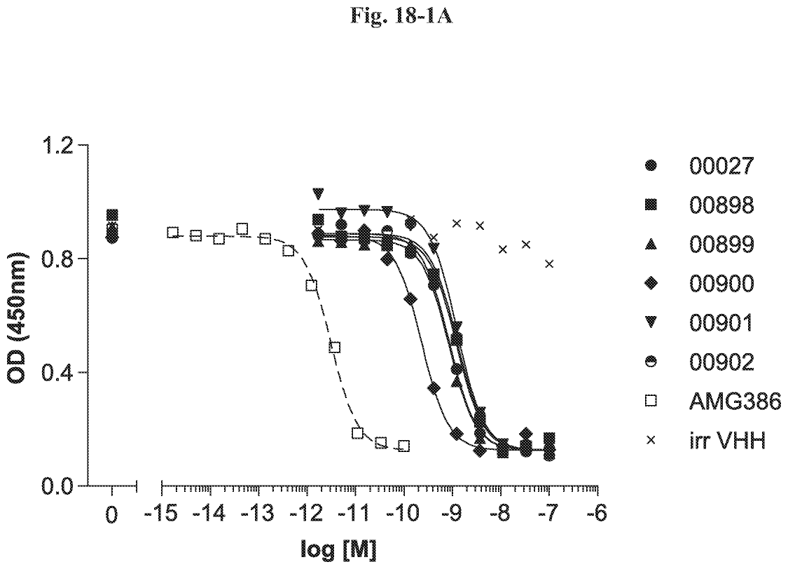

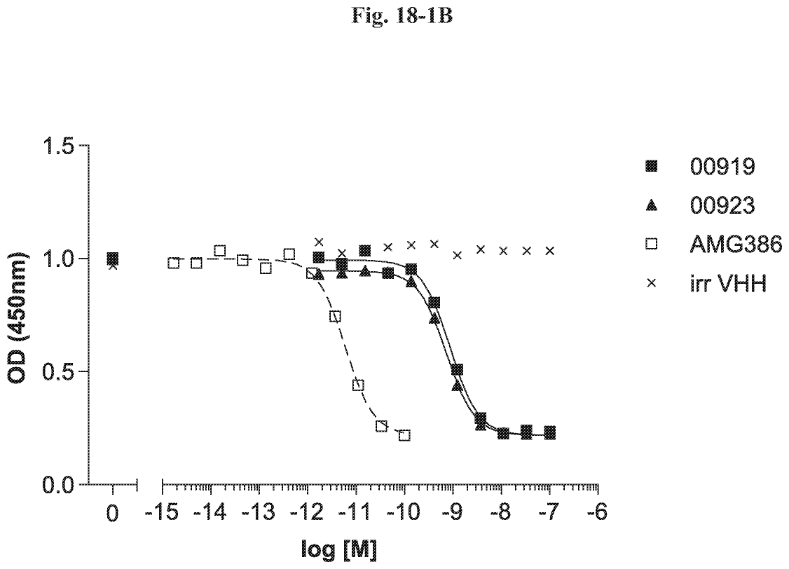

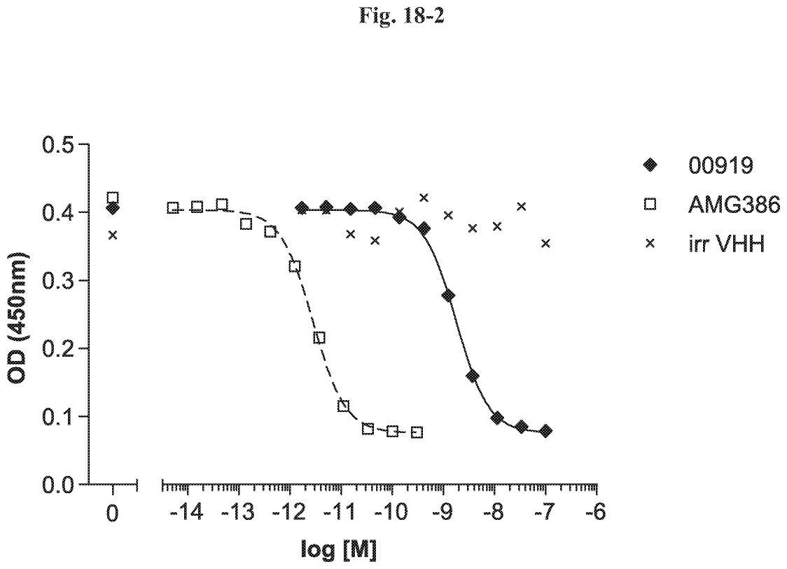

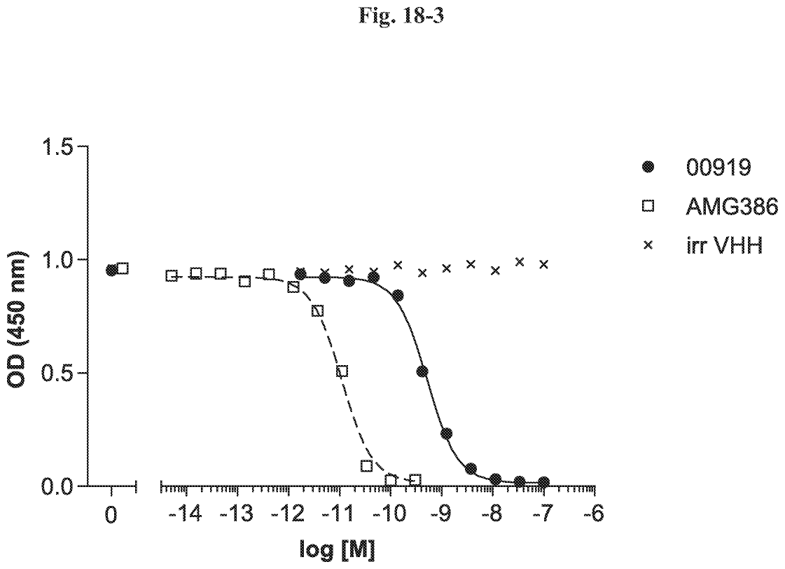

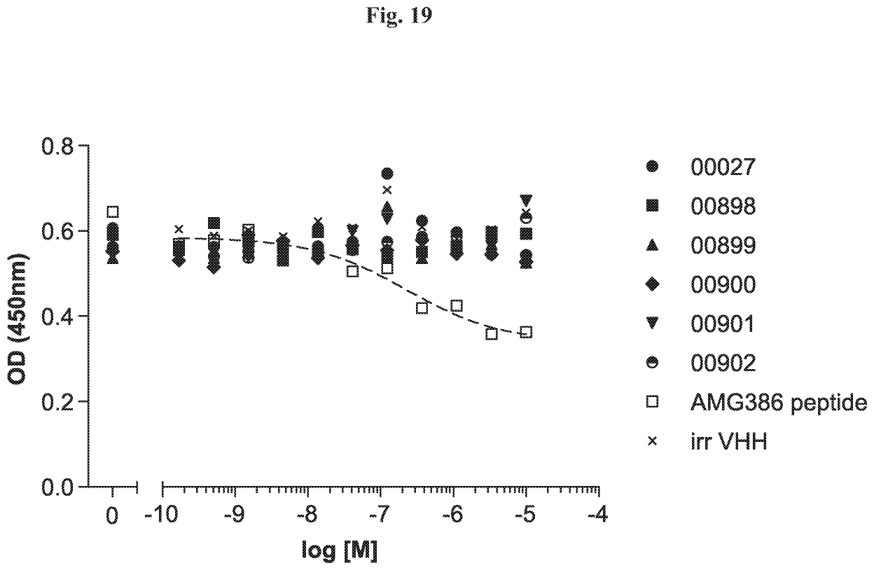

An additional set of variants (cycle 2) is created to explore the affinity maturation substitution on position A₂₄V and to further explore C₅₀X—S₅₃X variants (Table 21; FIG. 15-C; AA sequences are listed in Table 23-4-2). These variants are characterized as purified protein in the human (FIG. 18-1), mouse (FIG. 18-2) and cyno (FIG. 18-3) Ang2/Tie2 competition ELISA (Example 5.1; Example 5.2), the hAng1/hTie2 competition ELISA (Example 5.3; FIG. 19). Additionally, melting temperature (Tₘ) of each variant is determined in thermal shift assay (Example 6).

TABLE 21
Overview of Tm, IC50 (nM) in human, mouse and cyno Ang2
competition ELISA and hAng1/hAng2 IC50 ratios of cycle 2
sequence optimized variants of VHH 28D10

| | TSA | IC50 in Ang2-Tie2 ELISA | | | hAng1/ | % FR |
|---|---|---|---|---|---|---|
| | Tm (° C.) | hAng2 | mAng2 | cAng2 | hAng2 | identity |
| VHH ID | @ pH7 | (nM) | (nM) | (nM) | ratio | AbM |
| 28D10 | 66.1 | 1.8 | 5.7 | 2.1 | >5,675 | 86.5 |
| 00898 | 66.1 | 1.2 | n.d. | n.d. | >8,279 | 87.6 |
| 00899 | 61.9 | 0.9 | n.d. | n.d. | >11,749 | 87.6 |
| 00900 | 64.3 | 0.2 | n.d. | n.d. | >42,855 | 86.5 |
| 00901 | 67.3 | 1.2 | n.d. | n.d. | >8,054 | 87.6 |
| 00902 | 66.1 | 1.3 | n.d. | n.d. | >7,907 | 87.6 |
| 00919 | 67.3 | 0.9 | 1.8 | 0.7 | >11,212 | 91.0 |
| 00923 | 67.7 | 0.7 | n.d. | n.d. | n.d. | 89.9 |

n.d., not determined

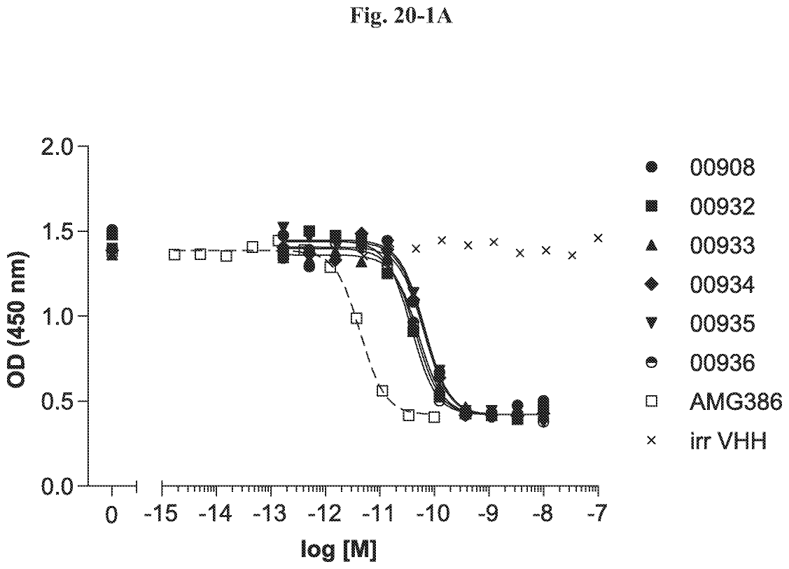

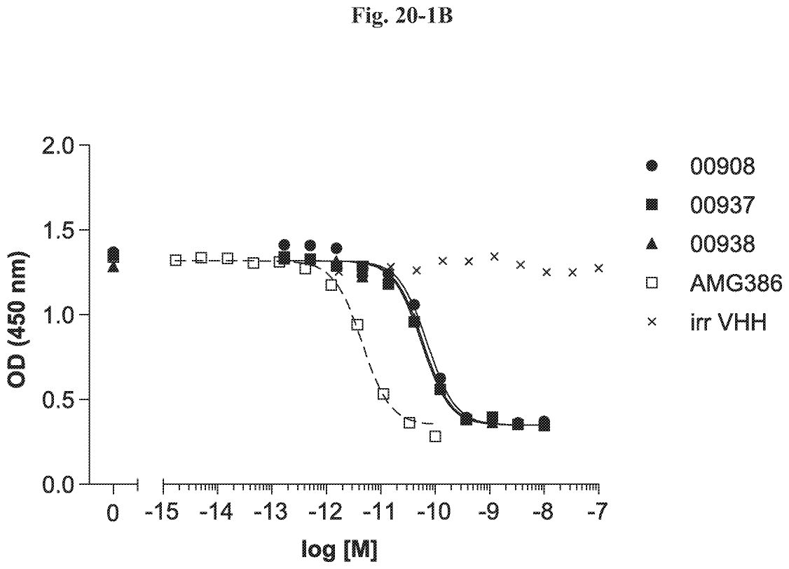

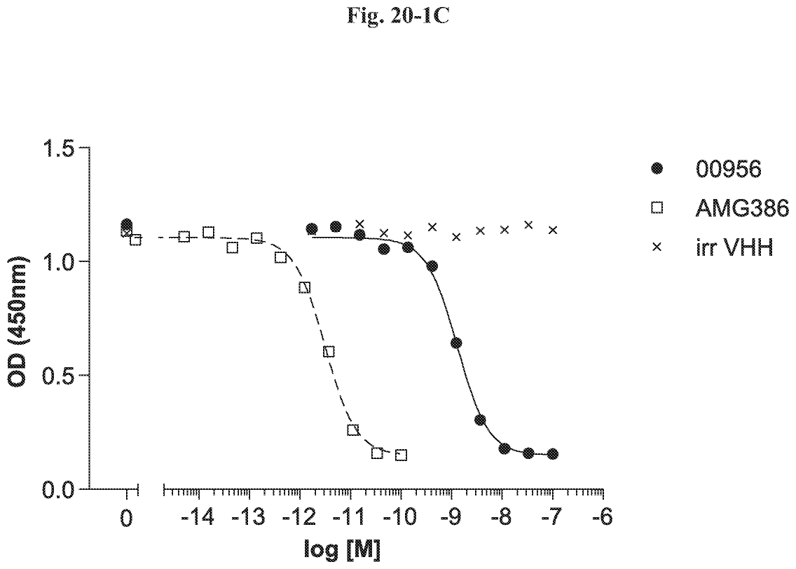

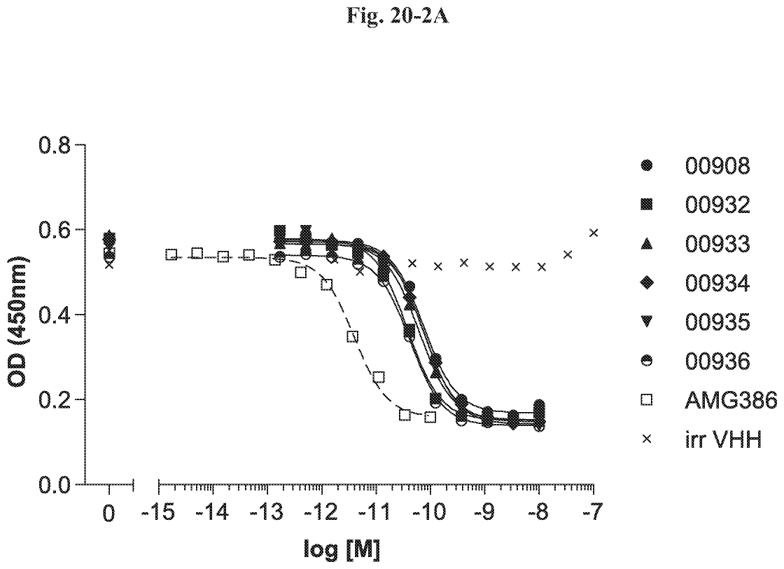

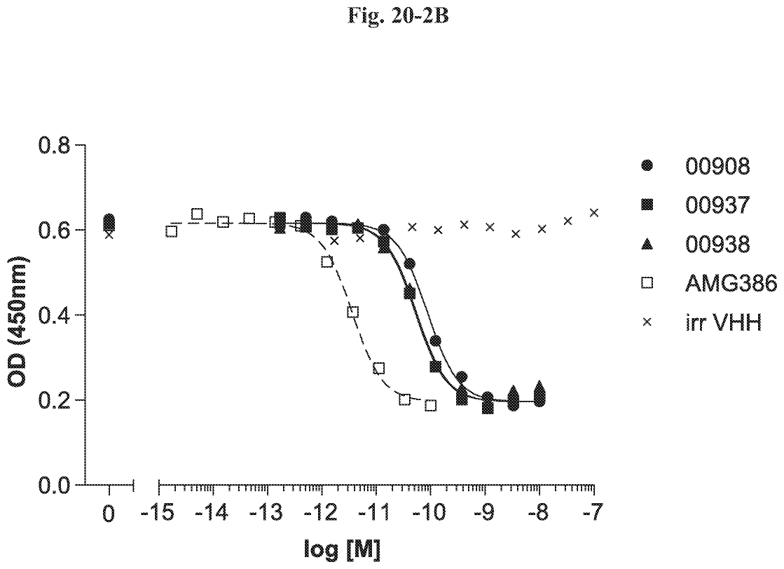

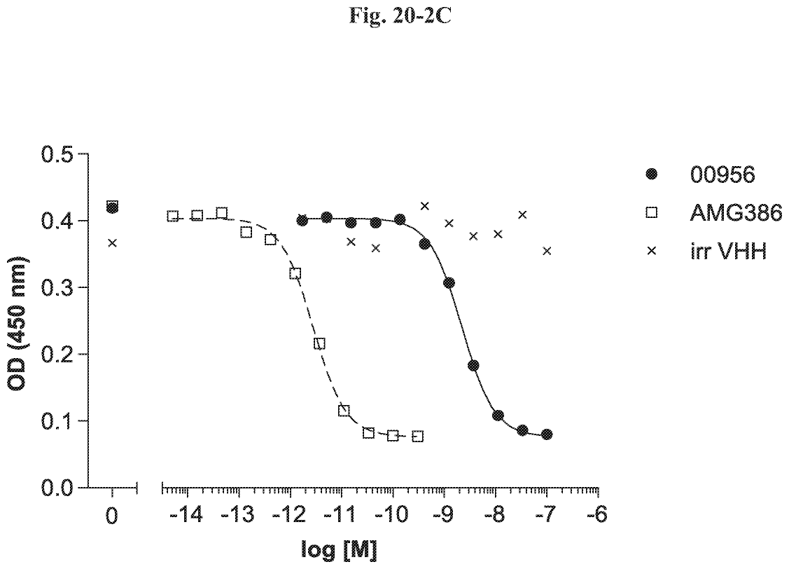

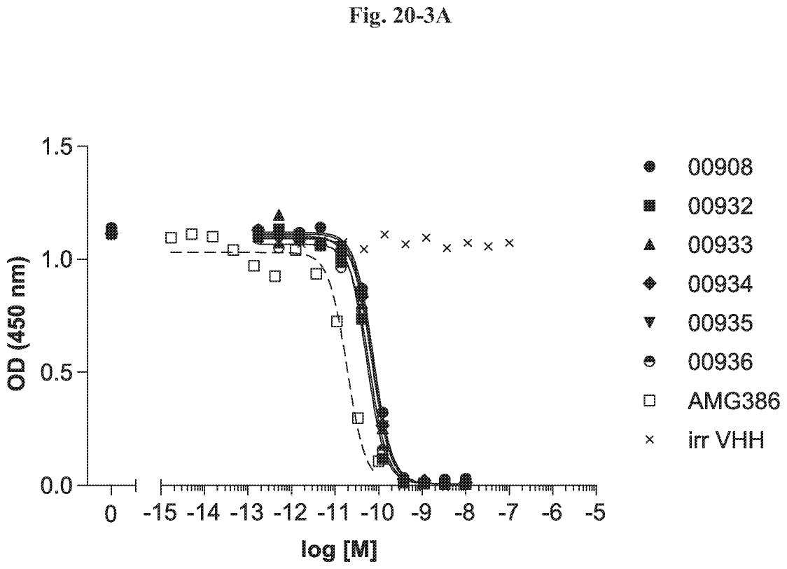

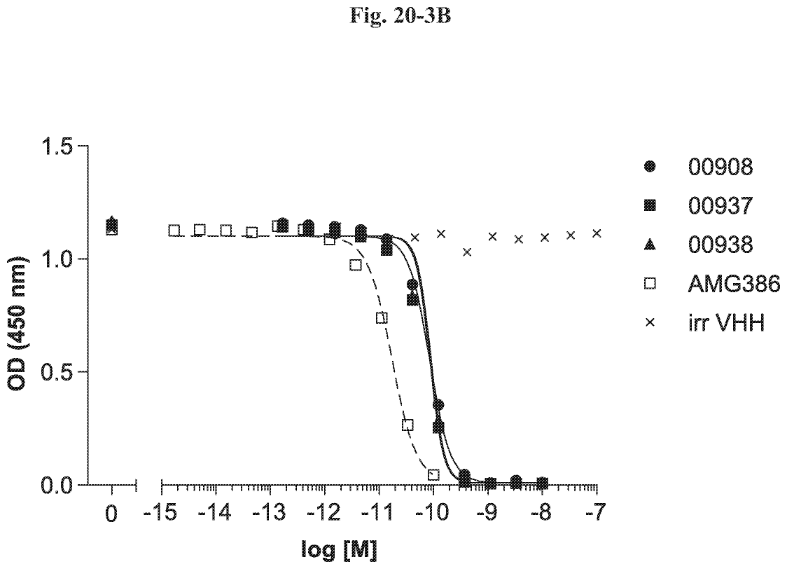

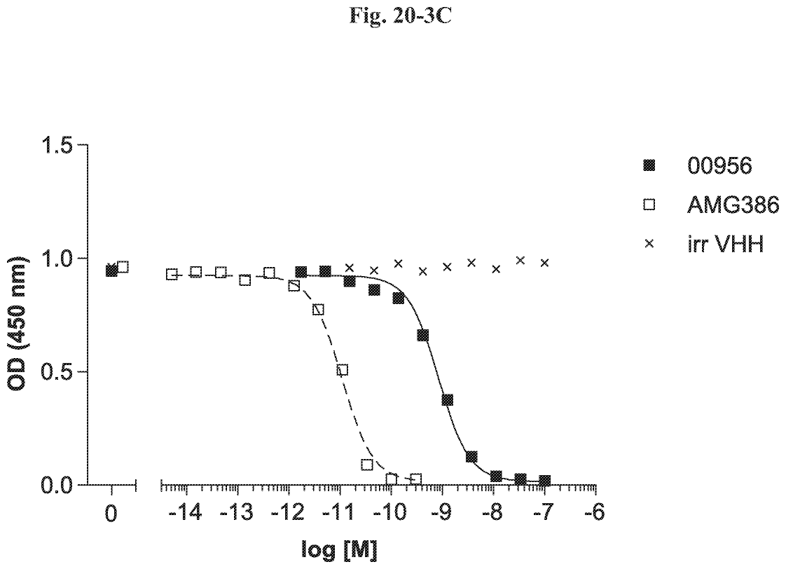

In parallel, a NNK library approach is used to knock out two post-translational modifications sites in CDR2: two sequential Asp isomerization sites on position D₅₂ₐS₅₃ and D₅₄G₅₅. Since D₅₄G is tolerated (Example 6; Table 20) no NNK approach was used to knock out this potential Asp isomerization site. In the end, 2 NNK libraries containing VHH clones carrying substitutions at positions D₅₂ₐ and S₅₃ to all other amino acids are screened in a hAng2/hTie2 competition AlphaScreen assay (Example 2). Briefly, periplasmic extracts containing expressed VHH are screened at 3 different dilutions (corresponding roughly to EC₂₀, EC₅₀ and EC₈₀ of the reference 00902) and changes in % inhibition at the different dilution points are compared to reference 00902. Based on the screening results and the data shown in Table 21, 7 additional VHH cycle 3 variants are constructed (based on 00908 backbone) (see FIG. 15-D). The final aim is to construct VHH variants that retain or show increased potency, increased thermostability and have relevant PTM sites knocked out compared to VHH 28D10. (AA sequences are listed in Table 24-3). These variants are characterized as purified protein in the human (FIG. 20-1), mouse (FIG. 20-2) and cyno (FIG. 20-3) Ang2/Tie2 competition ELISA (Example 5.1; Example 5.2), the hAng1/hTie2 competition ELISA (Example 5.3). Additionally, melting temperature (Tₘ) of each variant is determined in thermal shift assay (Example 6). An overview of the data can be found in Table 22. Additionally, % FR identity to the human germline is calculated. The most optimal sequence changes were finally applied to a non-affinity matured variant VHH 00956 (FIG. 15-D). Affinity of VHH 00919, 00938 and 00956 for human, mouse, cyno and rat Ang2 is shown in Table 23.

TABLE 22
Overview of Tₘ, IC₅₀ (nM) in human, mouse and cyno Ang2 competition ELISA and
hAng1/hAng2 IC₅₀ ratios of cycle 3 sequence optimized variants of VHH 28D10

| | TSA | IC50 in Ang2/Tie2 | | | | HUVEC | |
|---|---|---|---|---|---|---|---|
| | Tm @ | ELISA | | | hAng1/ | survival | % FR |
| | pH 7.0 | hAng2 | mAng2 | cAng2 | hAng2 | IC₅₀ | identity |
| VHH ID | (° C.) | (pM) | (pM) | (pM) | IC₅₀ ratio | (nM) | AbM |
| 00027 | 61.1 | 672 | 1,975 | 728 | >14,878 | n.d. | 87.6 |
| 00908 | 67.3 | 45 | 85 | 79 | >192,014 | n.d. | 91.0 |
| 00932 | 70.5 | 49 | 38 | 54 | >205,747 | n.d. | 89.9 |
| 00933 | 70.5 | 56 | 62 | 71 | >179,887 | n.d. | 91.0 |
| 00934 | 67.1 | 64 | 74 | 71 | >156,675 | n.d. | 91.0 |
| 00935 | 68.6 | 68 | 78 | 69 | >146,218 | n.d. | 91.0 |
| 00936 | 73.9 | 45 | 42 | 60 | >223,872 | n.d. | 89.9 |
| 00937 | 72.0 | 54 | 52 | 85 | >186,209 | n.d. | 89.9 |
| 00938 | 73.9 | 50 | 55 | 91 | >201,064 | 4.3 | 89.9 |
| 00956 | 68.0 | 1,300 | 2,200 | 830 | >7,727 | 6.8 | 91.0 |

n.d. not determined

TABLE 23
Affinity KD of purified VHHs 00919, 00938 and
00956 for recombinant human, cyno, mouse and rat Ang2

| | human Ang2-FLD | | | cyno Ang2-FLD | | |
|---|---|---|---|---|---|---|
| | kₐ | k_{d} | K_{D} | kₐ | k_{d} | K_{D} |
| | (1/Ms) | (1/s) | (M) | (1/Ms) | (1/s) | (M) |
| 00919 | 9.5E+05 | 1.5E−03 | 1.6E−09 | 2.3E+06 | 1.2E−03 | 5.4E−10 |
| 00938 | 1.6E+06 | 2.8E−05 | 1.7E−11 | 2.6E+06 | 2.2E−05 | 8.7E−12 |
| 00956 | 1.3E+06 | 1.5E−03 | 1.2E−09 | 1.7E+06 | 1.3E−03 | 7.2E−10 |
| | mouse Ang2-FLD | | | rat Ang2-FLD | | |
| | kₐ | k_{d} | K_{D} | kₐ | k_{d} | K_{D} |
| | (1/Ms) | (1/s) | (M) | (1/Ms) | (1/s) | (M) |
| 00919 | 1.3E+06 | 3.9E−03 | 3.0E−09 | 5.0E+05 | 5.0E−03 | 1.0E−08 |
| 00938 | 1.1E+06 | 5.6E−05 | 5.0E−11 | 5.1E+05 | 7.2E−05 | 1.4E−10 |
| 00956 | 9.8E+05 | 3.9E−03 | 4.0E−09 | 4.2E+05 | 5.2E−03 | 1.3E−08 |

TABLE 24-1

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00025/142 | EVQLVES | DYAI | WFRQA | TIRDSD | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00026/143 | EVQLVES | DYAI | WFRQA | AIRDSD | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00027/144 | EVQLVES | DYAI | WFRQA | SIRDND | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00041/145 | EVQLVES | DYAI | WFRQA | SIRDND | RFTISSDNAK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00042/146 | EVQLVES | DYAI | WFRQA | SIRDND | RFTISSDNSK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00043/147 | EVQLVES | DYAI | WFRQA | SIRDND | RFTISRDNAK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00048/148 | EVQLVES | DYAI | WFRQA | SIRDSD | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00052/149 | EVQLVES | DYAI | WFRQA | SIRDND | RFTISSDNDK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00053/150 | EVQLVES | DYAI | WFRQA | SIRDND | RFTISRDNDK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00054/151 | EVQLVES | DYAI | WFRQA | SIRDND | RFTISRDNSK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00055/152 | EVQLVES | DYAI | WFRQA | SIRDNG | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |

TABLE 24-2

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00898/153 | EVQLVES | DYAI | WFRQA | AIRDND | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00899/154 | EVQLVES | DYAI | WFRQA | SIRDIDG | RFTISSDNDK | VPAGRLRFG | WGQ |
| | GGGLVQA | G | PGKERE | STYYAD | NTVYLQMNSL | EQWYPLYE | GTLV |
| | GGSLRLS | | GVS | SVKG | KPEDTAVYYC | YDA | TVSS |
| | CAASGFT | | | | AA | | |
| | LD | | | | | | |
| 00900/155 | EVQLVES | DYAI | WFRQA | SIRDND | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAVSGFT | | | | AA | | |
| | LD | | | | | | |
| 00901/156 | EVQLVES | DYAI | WFRQA | AIRDSD | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |
| 00902/157 | EVQLVES | DYAI | WFRQA | AIRDSG | RFTISSDNDK | VPAGRLRFG | WGQ |
| | GGGLVQA | G | PGKERE | GSTYYA | NTVYLQMNSL | EQWYPLYE | GTLV |
| | GGSLRLS | | GVS | DSVKG | KPEDTAVYYC | YDA | TVSS |
| | CAASGFT | | | | AA | | |
| | LD | | | | | | |

TABLE 24-3

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| 00919/158 | EVQLVES | DYAI | WFRQA | AIRDNG | RFTISSDNSK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00923/159 | DVQLVES | DYAI | WFRQA | AIRDNG | RFTISSDNSK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |
| 00932/160 | EVQLVES | DYAI | WFRQA | SIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAVSGITL | | | | AA | | |
| | D | | | | | | |
| 00933/161 | EVQLVES | DYAI | WFRQA | AIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGITL | | | | AA | | |
| | D | | | | | | |
| 00934/162 | EVQLVES | DYAI | WFRQA | AIRESG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGITL | | | | AA | | |
| | D | | | | | | |
| 00935/163 | EVQLVES | DYAI | WFRQA | AIRSSG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAASGITL | | | | AA | | |
| | D | | | | | | |
| 00936/164 | EVQLVES | DYAI | WFRQA | AIRDNG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAVSGITL | | | | AA | | |
| | D | | | | | | |
| 00937/165 | EVQLVES | DYAI | WFRQA | AIRESG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAVSGITL | | | | AA | | |
| | D | | | | | | |
| 00938/166 | EVQLVES | DYAI | WFRQA | AIRSSG | RFTISSDNSK | VPAGRLRY | WGQ |
| | GGGLVQP | G | PGKERE | GSTYYA | NTVYLQMNSL | GEQWYPIYE | GTLV |
| | GGSLRLS | | GVS | DSVKG | RPEDTAVYYC | YDA | TVSS |
| | CAVSGITL | | | | AA | | |
| | D | | | | | | |
| 00956/167 | EVQLVES | DYAI | WFRQA | AIRSSG | RFTISSDNSK | VPAGRLRFG | WGQ |
| | GGGLVQP | G | PGKERE | GSTYYA | NTVYLQMNSL | EQWYPLYE | GTLV |
| | GGSLRLS | | GVS | DSVKG | RPEDTAVYYC | YDA | TVSS |
| | CAASGFT | | | | AA | | |
| | LD | | | | | | |

1. An Ang2-binding molecule comprising an immunoglobulin single variable domain, wherein said immunoglobulin single variable domain comprises three complementarity determining regions CDR1, CDR2 and CDR3, wherein CDR1 has an amino acid sequence selected from amino acid sequences shown in SEQ ID Nos: 168 to 170, CDR2 has an amino acid sequence selected from amino acid sequences shown in SEQ ID Nos: 171 to 173 and CDR3 has an amino acid selected from amino acid sequences shown in SEQ ID NOs: 174 to 177.

2. The Ang2-binding molecule according to claim 1, wherein
(a) CDR1 has an amino acid sequence shown in SEQ ID NO: 168, CDR2 has an amino acid sequence shown in SEQ ID NO: 171 and CDR3 has an amino acid sequence shown in SEQ ID NO: 174, or wherein
(b) CDR1 has an amino acid sequence shown in SEQ ID NO: 168, CDR2 has an amino acid sequence shown in SEQ ID NO: 171 and CDR3 has an amino acid sequence shown in SEQ ID NO: 175, or wherein
(c) CDR1 has an amino acid sequence shown in SEQ ID NO: 169, CDR2 has an amino acid sequence shown in SEQ ID NO: 172 and CDR3 has an amino acid sequence shown in SEQ ID NO: 176, or wherein
(d) CDR1 has an amino acid sequence shown in SEQ ID NO: 170, CDR2 has an amino acid sequence shown in SEQ ID NO: 173 and CDR3 has an amino acid sequence shown in SEQ ID NO: 177.

3. The Ang2-binding molecule according to claim 1, wherein said immunoglobulin single variable domain is a VHH or a domain antibody.

4. The Ang2-binding molecule according to claim 1, wherein said immunoglobulin single variable domain is a VHH.

5. The Ang2-binding molecule according to claim 4, wherein said VHH consists of an immunoglobulin single variable domain having a sequence selected from a group consisting of SEQ ID NOs: 167, 166, 129 and 138.

6. An Ang2-binding molecule consisting of the immunoglobulin single variable domain according to claim 5.

7. The Ang2-binding molecule according to claim 5 wherein said VHH consisting of an immunoglobulin single variable domain has a modification or exchange on N terminus, wherein said modification is a deletion of a first amino acid and said exchange is a replacement of the first aminom acid by another amino acid.

8. A nucleic acid molecule encoding the Ang2 binding molecule according to claim 1.

9. An expression vector comprising said nucleic acid molecule according to claim 8.

10. A host cell comprising one or more expression vectors according to claim 9.

11. A method for producing the Ang2-binding molecule according to claim 1, comprising the steps of:
(a) transfecting a host cell with one or more said vectors comprising said nucleic acid molecule encoding the Ang2 binding molecule according to claim 1,
(b) culturing said host cell, and
(c) recovering and purifying said Ang2 binding molecule.

12. A pharmaceutical composition comprising, as the active ingredient, one or more said Ang2-binding molecules according to claim 1, and at least a physiologically acceptable carrier.

13. The pharmaceutical composition according to claim 12, further comprising one or more additional therapeutic agents, selected from chemotherapeutic agents like DNA damaging agents and/or anti-mitotic drugs in cancer cells (e.g. taxol), or therapeutically active compounds that inhibit angiogenesis (an anti angiogenic drug such as anti VEGF/VEGF receptor inhibitor, e.g. avastin, nintedanib or sunitinib), or signal transduction pathway inhibitors such as mTOR inhibitors (e.g. temsirolimus), or a hormonal therapy agent (e.g. tamoxifen).

14. A method of treating a disease that is associated with Ang 2 mediated effects on angiogenesis comprising administering an effective amount of a pharmaceutical composition according to claim 12, to a patient in need thereof.

15. The method according to claim 14 wherein the disease is cancer and cancerous diseases selected from breast cancer, renal cell carcinoma, ovarian cancer and pancreatic cancer.

16. The method according to claim 14 wherein the disease is eye diseases selected from age-related macular degeneration and diabetic retinopathy.

17. The method according to claim 14 wherein the disease is chronic kidney disease, selected from diabetic nephropathy, postrenal failure, prerenal azotemia and intrinsic renal failure.

18. A method of treating a disease comprising administering to a patient in need, an effective amount of one or more Ang2-binding molecules according to claim 1 or pharmaceutical compositions thereof.
